(19) United States
(12) Patent Application Publication
(10) Pub. No.: US 2025/0268474 A1
(43) Pub. Date: Aug. 28, 2025

(54) Systems and Methods for Detecting Fibrosis

(71) Applicant: 3HELIX, INC., Salt Lake City, UT (US)

(72) Inventors: Lucas Bennink, Salt Lake City, UT (US); Michael Kirkness, Salt Lake City, UT (US); Peter Westenskow, Basel (CH); Markus Linder, Basel (CH)

(73) Assignee: 3HELIX, INC., Salt Lake City, UT (US)

(21) Appl. No.: 18/688,621

(22) PCT Filed: Sep. 1, 2022
(86) PCT No.: PCT/US2022/075819

§ 371 Date: Mar. 1, 2024

Related U.S. Application Data
(60) Provisional application No. 63/239,919, filed on Sep. 1, 2021.

(51) Int. Cl. A61B 5/00; A61K 49/00; C07K 14/00

(52) U.S. Cl. CPC A61B 5/0066; A61K 49/0032; A61K 49/0056; C07K 14/001

(57) ABSTRACT

The present disclosure provides a method comprising administering a labeled collagen hybridizing peptide (LCHP) to the subject, and imaging the LCHP, thereby detecting the presence or progression of fibrosis in the subject.

### BACKGROUND

Fibrosis is considered a deviation from homeostasis resulting in an overproduction of collagen and other ECM proteins. Fibrosis is a complex and dynamic system involving both the production and cleavage/remodeling of collagen. It has been shown that as fibrosis progresses there is an increase of collagen turnover. Anti-VEGF treatments can slow the vascularization associated with neovascular age-related macular degeneration (nAMD), resulting in improved vision in many of the patients. However, as many as 40% of patients develop subretinal-fibrosis after ten years of treatment with anti-VEGF for nAMD. Subretinal fibrosis is directly associated to the loss of vision. Current diagnostic tools lack the ability to monitor the fibrotic progression, especially in the early stages. The three most widely used tools are the Amsler grid method, fluorescein angiography, and optical coherence tomography (OCT).

The Amsler grid method focuses on changes in vision, which is done via a graph paper with a single dot that looks for the presence of wavy/curved lines. There is no molecular detection achieved through the Amsler grid method, but it can be used to monitor the progression of the disease.

Fluorescein angiography (FA) involves the systemic injection of fluorescein to monitor the vascularization, leaks, and changes in the vascular structure of the choroid via a fundus imaging. While FA allows the doctor to visualize the vascularization in 2D, it does not provide any structural information on the retina, including fibrosis.

OCT is a noninvasive method that allows for the visualization of the cross-sectional area of the retina including retinal thickening, subretinal fluid, and retinal pigment epithelium (RPE) damage. It should be noted that OCT does have challenges in detection and differentiation of fibrosis from drusen material, RPE changes, hemorrhage, retinal tissue, or Bruch's membrane.

Without a technique to monitor the subretinal fibrosis in nAMD patients, it will be a challenge to develop effective treatments for fibrosis and monitor the success of current therapeutics.

### SUMMARY OF THE DISCLOSURE

In one aspect, to solve the problem of detecting fibrosis, the present disclosure provides a method comprising administering a labeled collagen hybridizing peptide (LCHP) to the subject, and imaging the LCHP, thereby detecting the presence or progression of fibrosis in the subject.

In one aspect, to solve the problem of detecting fibrosis progression, the present disclosure provides a method comprising administering a labeled collagen hybridizing peptide (LCHP) to the subject, and imaging the LCHP, and administering another LCHP to the subject at another time point, and imaging said another LCHP, and comparing images from different time points, thereby detecting fibrosis progression in the subject.

In another aspect, the present disclosure further provides a method of diagnosing a fibrotic disease in a subject based on the presence of fibrosis in the subject, or the progression of fibrosis in the subject, as detected by the methods described herein.

In another aspect, the present disclosure further provides a method of treating a subject with a fibrotic disease, comprising diagnosing a fibrotic disease in a subject in accordance with a method provided herein, and administering an antifibrotic drug to the subject.

In another aspect, the present disclosure also provides a method of treating a subject with neovascular age-related macular degeneration (nAMD), comprising diagnosing nAMD in a subject in accordance with a method described herein and administering an antifibrotic drug to the subject.

### BRIEF DESCRIPTION OF THE DRAWINGS

FIG. 1 illustrates an overview of nAMD and fibrosis.

FIGS. 2A-2C illustrate current methods for nAMD diagnosis.

FIG. 3 illustrates a LCHP binding to unfolded collagen molecules.

FIGS. 4A and 4B illustrate visualizations of laser-induced choroidal neovascularization (CNV) and spontaneous CNV.

FIGS. 5A and 5B illustrate associated proteins of fibrosis and epithelial-mesenchymal transition (EMT). FIG. 5C shows increasing collagen remodeling as time progresses from four weeks up to 10 weeks in JR5558 mouse model.

FIGS. 6A-6B illustrate in vivo imaging using CHPs with Angiography (FA and IDCGA).

FIG. 6C illustrates that sCy7.5-CHP is sensitive enough to detect changes in the amount of damaged/denatured collagen as the laser power is increased to create the lesions in the eye. As the laser power increased, the amount of damaged collagen increased as evidenced by higher signal intensity from CHPs. This signal was quantified and there is a significant difference from the 500 mW level compared with the 150 mW and 300 mW power levels. At the power levels used, the laser was exposed for 100 ms.

FIG. 6D shows the histological analysis from lesions taken from LCNV eyes at 150, 300, and 500 mW power levels. The images on the left show staining of fibronectin (purple) and LCHPs (red). Fibronectin is a common ECM protein stain, while LCHPs show the damaged collagen. The graphics on the right show how the signal seen in histology correlates to the in vivo signal seen in FIG. 6C. As shown, there is good agreement between in vivo LCHP signal and the fibronectin (r=0.69) and even better agreement with the in vivo signal and histological CHP signal (r=0.76). Together, this validates that the in vivo signal can be a reliable indicator for neovascularization due to nAMD.

FIG. 7 shows the fibrotic response and resolution to the LCNV mouse model one week after and eight weeks after laser injury. The schematic above shows the timeline for laser injury, CHP injections, and imaging. The in vivo images show CHP signal one week post laser injury and eight weeks post laser injury. When quantified, the graphs to the right show significant differences between the collagen remodeling in week one vs. week eight when examined in vivo and ex vivo.

FIG. 8A illustrates the therapeutic effect of a bi-specific anti-VEGF/ANG-2 antibody treatment (anti-VA2) for nAMD compared with a generic IgG antibody in JR5558 mice. Schematic A highlights the timing of injections and CHP imaging for the study. Panel B shows the in vivo imaging results of each treatment while panel C shows the signal quantification using CHPs. Panel D compares the CHP signal intensity in each treatment group to fibronectin (a common marker of fibrosis)

FIG. 8B shows the ex vivo histological analysis from lesions taken after treatment with anti-VA2 compared to IgG treatment. CHP signal (red) was compared with fibronectin staining (purple). Fibronectin is a common ECM protein stain, while LCHPs show the damaged collagen. The graphs below show how the signal seen in histology correlates to the in vivo signal seen in FIG. 8A. As shown, there is good agreement between in vivo LCHP signal and the fibronectin (r=0.54) and even better agreement with the in vivo signal and histological CHP signal (r=0.63). Together, this validates that the in vivo signal can be a reliable indicator for therapeutic efficacy on novel drug treatments for neovascularization and fibrosis due to nAMD.

FIG. 9 illustrates CHP histology in non-human primates after laser induced with CNV.

### DETAILED DESCRIPTION

Hereinafter, exemplary embodiments of the present disclosure will be described in detail. However, the present disclosure is not limited to the embodiments disclosed below, but may be implemented in various forms. The following embodiments are described in order to enable those of ordinary skill in the art to embody and practice embodiments of the present disclosure.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide conjugate is disclosed and discussed and a number of modifications that can be made to a number of molecules including the peptide conjugate are discussed, each and every combination and permutation of the peptide conjugate and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### Definitions

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

It must be noted that as used herein and in the appended claims, the singular forms “a”, “an”, and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth. Similarly, the word “or” is intended to include “and” unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary unless a particular order is clearly indicated by the context.

As used herein, the term “about” refers to a numeric value, including, for example, whole numbers, fractions, and percentages, whether or not explicitly indicated. The term “about” generally refers to a range of numerical values (e.g., +/−5, 6, 7, 8, 9 or 10% of the recited value) that one of ordinary skill in the art would consider equivalent to the recited value (e.g., having the same function or result). In some instances, the term “about” may include numerical values that are rounded to the nearest significant figure.

As used herein “collagen” can be from any tissue type (e.g., bone, dermis, tendon, ligaments, etc.). Collagen can refer to a molecule in which three alpha chains of polyproline II-like structure fold together into a triple helix. Additionally, this can apply to any protein that contains a triple-helical region including collagen types I-XXVIII and bacterial collagen. The term “collagen” as used herein can refer to all forms of collagen, including artificial collagen and collagen which has been processed or otherwise modified. In some embodiments, the collagen is selected from type I collagen, type II collagen, type III collagen, type IV collagen, type V collagen, type VI collagen, type VII collagen, type VIII collagen, type IX collagen, type X collagen, type XI collagen, type XII collagen, type XIII collagen, type XIV collagen, type XV collagen, type XVI collagen, type XVII collagen, type XVIII collagen, type XIX collagen, type XX collagen, type XXI collagen, type XXII collagen, type XXIII collagen, type XXIV collagen, type XXV collagen, type XXVI collagen, type XXVII collagen, type XXVIII collagen, and a combination thereof.

As used herein, the term “proline or modified proline” means the amino acid proline and various isomers, analogs and variants thereof, including both natural and non-natural isomers. Examples of modified proline include, without limitation, hydroxyproline, 4-fluoro proline, and 4-chloroproline.

In some embodiments, the method excludes collecting a sample from the subject. In some embodiments, “subject” herein can refer to a human or an animal or bacteria or cell cultures from any of the aforementioned groups. Non-limiting examples of animals include vertebrates such as a primate, a rodent, a domestic animal, or a game animal. Primates include chimpanzees, cynomolgus monkeys, spider monkeys, and macaques (e.g., Rhesus). Rodents include mice, rats, woodchucks, ferrets, rabbits, and hamsters. Domestic and game animals include cows, horses, pigs, deer, bison, buffalo, moose, feline species (e.g., domestic cat), and canine species (e.g., dog, fox, wolf). Fish including Chondrichthyes (cartilaginous fishes) and Osteichthyes (bony fishes). The subject may be mammal. The mammal can be a human, non-human primate, mouse, rat, dog, cat, horse, or cow, but are not limited to these examples. In addition, the methods described herein can be used to diagnose and/or treat domesticated animals or pets. The term does not denote a particular age or sex. Thus, adult and newborn subjects, as well as fetuses, whether male or female, are intended to be included within the scope of this term.

The term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a disease or injury involving collagen damage can refer to reducing or eliminating the amount of damaged/denatured collagen. Treatment can also be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

### I. Introduction

With reference to the appended drawings, exemplary embodiments of the present disclosure will be described in detail below. To aid in understanding the present disclosure, like numbers refer to like elements throughout the description of the figures, and the description of the same elements will be not reiterated.

### II. Fibrosis

Fibrosis is considered a deviation from homeostasis resulting in an overproduction of collagen and other ECM proteins. Fibrosis is a complex and dynamic system involving both the production and cleavage/remodeling of collagen. It has been shown that as fibrosis progresses there is an increase of collagen turnover.

### III. Detection of Fibrosis

In one aspect, the present disclosure provides a method of detecting fibrosis in a subject, comprising administering a labeled collagen hybridizing peptide (LCHP) to the subject, and imaging the LCHPs in-vivo, thereby detecting presence or progression of fibrosis in the subject. In an exemplary embodiment, the fibrosis is subretinal fibrosis. In an exemplary embodiment, the subject is human.

IIIa. Labeled Collagen Hybridizing Peptide (LCHP)

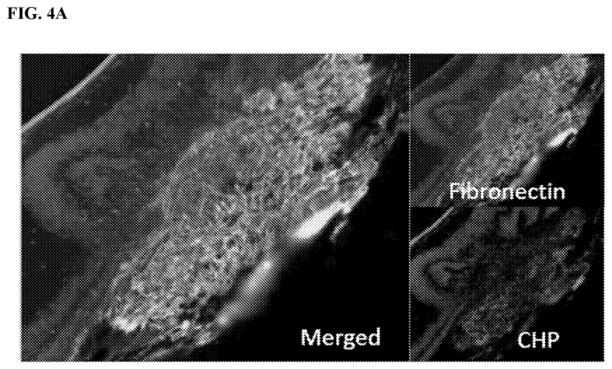

Collagen is the most abundant protein in a human body and is a critical component of almost all organs and tissues, providing the framework for cell attachment and growth. All types of collagen from all species share the triple helical protein structure, which is nearly exclusively found in collagen. After being cleaved by a collagenase, the collagen molecule becomes thermally unstable at body temperature and the triple helix spontaneously denatures. The unfolding of the collagen triple helix occurs during mechanical injuries, burns (chemical or thermal), or abrasions. The CHP described herein may specifically bind to unfolded collagen molecules by forming a triple helix with the denatured alpha-chains of collagen, in a fashion analogous to a primer binding to a melted DNA strand during PCR. Conjugated with a detection moiety, CHP may enable direct detection of unfolded collagen molecules in fibrosis that are undergoing active collagen remodeling. FIG. 3 illustrates a LCHP binding to unfolded collagen molecules.

As used herein, “labeled collagen hybridizing peptide” or LCHP can refer to a molecule represented by Formula I:

L-Sₘ-(Gly-X-Y)ₙ-Tⱼ Formula I

in which L is one or more detection moieties, S is a spacer moiety, m is an integer from 0 to 25, Gly is glycine, X is an amino acid, Y is an amino acid, n is an integer from 3 to 20, and T is a terminus moiety, j is an integer from 0 to 1, wherein at least one of X and Y is proline or modified proline. Examples of modified proline include hydroxyproline, 4-fluoro proline, and 4-chloroproline. In some embodiments, the modified proline is hydroxyproline. In some embodiments, each of X and Y is independently proline or modified proline, for example, proline or hydroxyproline. In some embodiments, X and Y are proline and hydroxyproline, respectively. In some embodiments, X and Y are 2S, 4S-4-fluoroproline and hydroxyproline, respectively. In some embodiments, X and Y are 2S, 4S-4-chloroproline and hydroxyproline, respectively.

Fluorescent detection moieties can include dyes chosen for immunofluorescence that are excited by light of one wavelength (e.g., blue or green) and emit light of a different wavelength in the visible spectrum. Exemplary detection moieties are fluorescein, which emits green light, Texas Red and Peridinin chlorophyll protein (PerCP), which emit red light, and rhodamine and phycoerythrin (PE) which emit orange/red light. By using selective filters, only the light coming from the detection moiety used is detected in the fluorescence microscope.

In some embodiments, the detection moiety is detected at a wavelength from 340 nm to 800 nm. In some embodiments, the detection moiety is a near-infrared (NIR) dye.

In an exemplary embodiment, the detection moiety is selected from the group consisting of ALEXA FLUOR dyes, cyanine dyes, sulfo-cyanine dyes, indocyanine dyes, TIDE FLUOR dyes, TAMRA, FITC, 5-FAM, carboxyfluorescein, coumarin dyes, and rhodamine dyes. These detection moieties can be purchased from Sigma Aldrich, ThermoFisher, AbCam, etc. Specifically, sulfo-cyanine dyes can be purchased from Lumiprobe, TIDE FLUOR dyes can be purchased from AAT Bioquest or BaChem. In some embodiments, the detection moiety is a gold nanoparticle. Gold nanoparticles can be purchased from Nanopartz, Sigma Aldrich, Particle-Works, etc. In some embodiments, the gold particle size is from about 1 nm to about 15.2 microns. In some embodiments, the gold particle shape is spherical. In some embodiments, the gold particle shape is a nanorod shape. In some embodiments, the detection moiety is an iron oxide nanoparticle. In some embodiments, the detection moiety is a radiolabel. In some embodiments, the radiolabel is selected from the group consisting of technetium, indium-111, copper-64, yttrium-86, fluorine-18, and zirconium-89.

In some embodiments, the detection moiety is prednisolone acetate. In some embodiments, the detection moiety is triamcinolone acetonide. In some embodiments, the detection moiety is a lipid-based artificial tear.

In some embodiments, the detection moiety is an ALEXA FLUOR dye. In additional embodiments, the AlexaFluor dye is selected from a group consisting of: Alexa Fluor 350, Alexa Fluor 405, Alexa Fluor 430, Alexa Fluor 488, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750, and Alexa Fluor 790.

In some embodiments, the detection moiety is a cyanine dye. In additional embodiments, the cyanine dye is selected from a group consisting of: Cy3, Cy3.5, Cy5, Cy5.5, Cy7, and Cy7.5.

In some embodiments, the detection moiety is a sulfonated-cyanine dye (sulfo-cyanine). In additional embodiments, the sulfo-cyanine dye is selected from a group consisting of: sCy3, sCy3.5, sCy5, sCy5.5, sCy7, and sCy7.5

In some embodiments, the detection moiety is a TIDE FLUOR dye. In additional embodiments, the TIDE FLUOR dye is selected from a group consisting of: TF1, TF2, TF3WS, TF3, TF4, TF5WS, TF6WS, TF7WS, and TF8WS.

In some embodiments, the dye described herein may be attached to the CHP via a conjugation chemistry selected from the group consisting of: NHS-ester, maleimide-thiol, azide, hydrazides, alkynes, carboxylic acids, and amine/amino. Methods of dye conjugation are described in Bioconjugate Techniques, 3^{rd} Ed., Greg T. Hermanson, Academic Press (2013).

In an embodiment, S is an amino acid. In an exemplary embodiment, m is 0 or 1 or 2 or 3 or 4 or 5 or 6 or 7 or 8 or 9 or 10. In an embodiment, S is glycine. In an exemplary embodiment, m is 0 or 1 or 2 or 3 or 4 or 5. In an exemplary embodiment, m is 3. In an exemplary embodiment, Sₘ is GlyGlyGly. In an embodiment, S is Ahx (alternatively known as aminocaproic acid, or 6-aminohexanoic acid). In an exemplary embodiment, m is 1. In an exemplary embodiment, Sₘ is Ahx. In an embodiment, S is ethylene glycol. In an embodiment, Sₘ is (OCH₂CH₂)₁₋₄.

In an exemplary embodiment, n is 3. In an exemplary embodiment, n is 4. In an exemplary embodiment, n is 5. In an exemplary embodiment, n is 6. In an exemplary embodiment, n is 7. In an exemplary embodiment, n is 8. In an exemplary embodiment, n is 9. In an exemplary embodiment, n is 10. In an exemplary embodiment, n is 11. In an exemplary embodiment, n is 12. In an exemplary embodiment, n is 13. In an exemplary embodiment, n is 14. In an exemplary embodiment, n is 15. In an exemplary embodiment, n is 16. In an exemplary embodiment, n is 17. In an exemplary embodiment, n is 18. In an exemplary embodiment, n is 19. In an exemplary embodiment, n is 20.

In an exemplary embodiment, the modified proline is fluoroproline. In an exemplary embodiment, the modified proline is 2S,4R-4-fluoroproline (trans-fluoroproline). In an exemplary embodiment, the modified proline is 2S,4S-4-fluoroproline (cis-fluoroproline). In an exemplary embodiment, the modified proline is chloroproline. In an exemplary embodiment, the modified proline is 2S,4S-4-chloroproline (cis-chloroproline). In an exemplary embodiment, the modified proline is methylproline. In an exemplary embodiment, the modified proline is 2S,4S-4-methylproline (cis-methylproline).

In an exemplary embodiment, the modified proline is hydroxyproline. In an exemplary embodiment, the modified proline is 2S, 4R-trans hydroxyproline. In an exemplary embodiment, the modified proline is t-butoxyproline. In an exemplary embodiment, the modified proline is N-α-Fmoc-O-t.-butyl-L-trans-4-hydroxyproline, or Fmoc-Hyp(tBu)-OH.

In an exemplary embodiment, T is methyl. In an exemplary embodiment, T is H. In an exemplary embodiment, T is COOH. In an exemplary embodiment, T is NH₂. Additional options for T can be found here: pepscan.com/custom-peptide-synthesis/peptide-modifications/c-terminal-modifications/.

In an exemplary embodiment, the LCHP is L-Sₘ-(Gly-X-Hyp)ₙ-H, in which L, S, m, X, and n are as described herein, Gly is glycine, and Hyp is trans-hydroxyproline. In an exemplary embodiment, the LCHP is L-Sₘ-(Gly-Pro-Hyp)ₙ-H, in which L, S, m, and n are as described herein, Gly is glycine, Pro is proline, and Hyp is trans-hydroxyproline. In an exemplary embodiment, the LCHP is L-Sₘ-(Gly-X-Hyp)ₙ-H, in which L, S, m, and n are as described herein, Gly is glycine, X is fluoroproline, and Hyp is trans-hydroxyproline. In an exemplary embodiment, the LCHP is L-Sₘ-(Gly-X-Hyp)ₙ-H, in which L, S, m, and n are as described herein, Gly is glycine, X is cis-fluoroproline, and Hyp is trans-hydroxyproline. In an exemplary embodiment, the LCHP is L-Sₘ-(Gly-X-Hyp)ₙ-H, in which L, S, m, and n are as described herein, Gly is glycine, X is trans-fluoroproline, and Hyp is trans-hydroxyproline. In an exemplary embodiment, the LCHP is L-GGG-(Gly-X-Hyp)ₙ-H, in which L, S, m, X, and n are as described herein, Gly is glycine, X is trans-fluoroproline, and Hyp is trans-hydroxyproline. In an exemplary embodiment, the LCHP can be L-S-(Gly-X-Hyp)₉, wherein L, S, and X are as described herein, X is proline or modified proline, Gly is glycine, and Hyp is trans-hydroxyproline.

In some embodiments, the LCHP described herein comprises a sequence represented by Formula II:

L-S-(Gly-X-Y)ₙ-T Formula II

in which L, S, n, X, Y, and T are as described herein.

In some embodiments, the LCHP described herein comprises a sequence represented by Formula III:

L-S-(Gly-X-Y)ₙ-(Gly-A-B)ₚ-(Gly-X-Y)_{q} Formula III

in which L, S, n, X, and Y are as described herein, A and B may be independently any amino acid, p is an integer from 1 to 20, and q is an integer from 1 to 20. In an exemplary embodiment, p is 2. In an exemplary embodiment, p is 3. In an exemplary embodiment, p is 4. In an exemplary embodiment, p is 5. In an exemplary embodiment, p is 6. In an exemplary embodiment, p is 7. In an exemplary embodiment, p is 8. In an exemplary embodiment, p is 9. In an exemplary embodiment, p is 10. In an exemplary embodiment, p is 11. In an exemplary embodiment, p is 12. In an exemplary embodiment, p is 13. In an exemplary embodiment, p is 14. In an exemplary embodiment, p is 15. In an exemplary embodiment, p is 16. In an exemplary embodiment, p is 17. In an exemplary embodiment, p is 18. In an exemplary embodiment, p is 19. In an exemplary embodiment, p is 20. In an exemplary embodiment, q is 2. In an exemplary embodiment, q is 3. In an exemplary embodiment, q is 4. In an exemplary embodiment, q is 5. In an exemplary embodiment, q is 6. In an exemplary embodiment, q is 7. In an exemplary embodiment, q is 8. In an exemplary embodiment, q is 9. In an exemplary embodiment, q is 10. In an exemplary embodiment, q is 11. In an exemplary embodiment, q is 12. In an exemplary embodiment, q is 13. In an exemplary embodiment, q is 14. In an exemplary embodiment, q is 15. In an exemplary embodiment, q is 16. In an exemplary embodiment, q is 17. In an exemplary embodiment, q is 18. In an exemplary embodiment, q is 19. In an exemplary embodiment, q is 20.

In some embodiments, the LCHP described herein comprises a sequence represented by L-S-(Gly-X-Y)₃, a sequence represented by L-S-(Gly-X-Y)₄, a sequence represented by L-S-(Gly-X-Y)₅, a sequence represented by L-S-(Gly-X-Y)₆, a sequence represented by L-S-(Gly-X-Y)₇, a sequence represented by L-S-(Gly-X-Y)₈, a sequence represented by L-S-(Gly-X-Y)₉, a sequence represented by L-S-(Gly-X-Y)₁₀, a sequence represented by L-S-(Gly-X-Y)₁₁, a sequence represented by L-S-(Gly-X-Y)₁₂, a sequence represented by L-S-(Gly-X-Y)₁₃, a sequence represented by L-S-(Gly-X-Y)₁₄, a sequence represented by L-S-(Gly-X-Y)₁₅, a sequence represented by L-S-(Gly-X-Y)₁₆, a sequence represented by L-S-(Gly-X-Y)₁₇, a sequence represented by L-S-(Gly-X-Y)₁₈, a sequence represented by L-S-(Gly-X-Y)₁₉, or a sequence represented by L-S-(Gly-X-Y)₂₀, in which L, S, X and Y are as described herein, and Gly is glycine.

In some embodiments, the LCHP described herein comprises any one of amino acid sequences of the sequence identifiers or the labeled sequences shown in Table 1 below.

TABLE 1
Sequences for Labeled Collagen Hybridizing Peptides

| Identifier | Labeled Sequence (One-Letter Amino Acid Symbols) |
|---|---|
| SEQ ID NO: 1 | Biotin-GGG-GPOGPOGPOGPOGPOGPOGPOGPOGPO-CONH2 |
| SEQ ID NO: 2 | 5(6)-Carboxyfluorescein-GGG-GPOGPOGPOGPOGPOGPOGPOGPOGPO- |
| | CONH₂ |
| SEQ ID NO: 3 | Alexa Fluor-350-GGG-GPOGPOGPOGPOGPOGPOGPOGPOGPO-CONH₂ |
| SEQ ID NO: 4 | Alexa Fluor-430-GGG-GPOGPOGPOGPOGPOGPOGPOGPOGPO-CONH₂ |
| SEQ ID NO: 5 | Alexa Fluor-488-GGG-GPOGPOGPOGPOGPOGPOGPOGPOGPO-CONH₂ |
| SEQ ID NO: 6 | Alexa Fluor-546-GGG-GPOGPOGPOGPOGPOGPOGPOGPOGPO-CONH₂ |
| SEQ ID NO: 7 | Alexa Fluor-568-GGG-GPOGPOGPOGPOGPOGPOGPOGPOGPO-CONH₂ |
| SEQ ID NO: 8 | Alexa Fluor-594-GGG-GPOGPOGPOGPOGPOGPOGPOGPOGPO-CONH₂ |
| SEQ ID NO: 9 | Alexa Fluor-633-GGG-GPOGPOGPOGPOGPOGPOGPOGPOGPO-CONH₂ |
| SEQ ID NO: 10 | Alexa Fluor-660-GGG-GPOGPOGPOGPOGPOGPOGPOGPOGPO-CONH₂ |
| SEQ ID NO: 11 | Alexa Fluor-680-GGG-GPOGPOGPOGPOGPOGPOGPOGPOGPO-CONH₂ |
| SEQ ID NO: 12 | Cyanine 3-GGG-GPOGPOGPOGPOGPOGPOGPOGPOGPO-CONH₂ |
| SEQ ID NO: 13 | Cyanine 5-GGG-GPOGPOGPOGPOGPOGPOGPOGPOGPO-CONH₂ |
| SEQ ID NO: 14 | Cyanine 5.5-GGG-GPOGPOGPOGPOGPOGPOGPOGPOGPO-CONH2 |
| SEQ ID NO: 15 | Cyanine 7-GGG-GPOGPOGPOGPOGPOGPOGPOGPOGPO-CONH₂ |
| SEQ ID NO: 16 | Cyanine 7.5-GGG-GPOGPOGPOGPOGPOGPOGPOGPOGPO-CONH₂ |
| SEQ ID NO: 17 | Cyanine 3-phycoerythrin-GGG-GPOGPOGPOGPOGPOGPOGPOGPOGPO- |
| | CONH₂ |
| SEQ ID NO: 18 | Cyanine 5-phycoerythrin-GGG-GPOGPOGPOGPOGPOGPOGPOGPOGPO- |
| | CONH₂ |
| SEQ ID NO: 19 | Cyanine 5.5-phycoerythrin-GGG-GPOGPOGPOGPOGPOGPOGPOGPOGPO- |
| | CONH₂ |
| SEQ ID NO: 20 | Cyanine 7-phycoerythrin-GGG-GPOGPOGPOGPOGPOGPOGPOGPOGPO- |
| | CONH₂ |
| SEQ ID NO: 21 | Cyanine 7.5-phycoerythrin-GGG-GPOGPOGPOGPOGPOGPOGPOGPOGPO- |
| | CONH₂ |
| SEQ ID NO: 22 | Rhodamine-GGG-GPOGPOGPOGPOGPOGPOGPOGPOGPO-CONH₂ |
| SEQ ID NO: 23 | Sulfo-Cyanine3-GGG-GPOGPOGPOGPOGPOGPOGPOGPOGPO-CONH₂ |
| SEQ ID NO: 24 | Sulfo-Cyanine5-GGG-GPOGPOGPOGPOGPOGPOGPOGPOGPO-CONH₂ |
| SEQ ID NO: 25 | Sulfo-Cyanine5.5-GGG-GPOGPOGPOGPOGPOGPOGPOGPOGPO-CONH₂ |
| SEQ ID NO: 26 | Sulfo-Cyanine7-GGG-GPOGPOGPOGPOGPOGPOGPOGPOGPO-CONH₂ |
| SEQ ID NO: 27 | Sulfo-Cyanine7.5-GGG-GPOGPOGPOGPOGPOGPOGPOGPOGPO-CONH₂ |
| SEQ ID NO: 28 | Texas Red-GGG-GPOGPOGPOGPOGPOGPOGPOGPOGPO-CONH₂ |
| SEQ ID NO: 29 | Biotin-GGG-GfOGfOGfOGfOGfOGfOGfOGfOGfO-CONH₂ |
| SEQ ID NO: 30 | 5(6)-Carboxyfluorescein-GGG-GfOGfOGfOGfOGfOGfOGfOGfOGfO-CONH₂ |
| SEQ ID NO: 31 | Alexa Fluor-350-GGG-GfOGfOGfOGfOGfOGfOGfOGfOGfO-CONH₂ |
| SEQ ID NO: 32 | Alexa Fluor-430-GGG-GfOGfOGfOGfOGfOGfOGfOGfOGfO-CONH₂ |
| SEQ ID NO: 33 | Alexa Fluor-488-GGG-GfOGfOGfOGfOGfOGfOGfOGFOGO-CONH₂ |
| SEQ ID NO: 34 | Alexa Fluor-546-GGG-GfOGfOGfOGfOGfOGfOGfOGfOGfO-CONH₂ |
| SEQ ID NO: 35 | Alexa Fluor-568-GGG-GfOGfOGfOGfOGfOGfOGfOGfOGfO-CONH₂ |
| SEQ ID NO: 36 | Alexa Fluor-594-GGG-GfOGfOGfOGfOGfOGfOGfOGfOGfO-CONH₂ |
| SEQ ID NO: 37 | Alexa Fluor-633-GGG-GfOGfOGfOGfOGfOGfOGfOGfOGfO-CONH₂ |
| SEQ ID NO: 38 | Alexa Fluor-660-GGG-GfOGfOGfOGfOGfOGfOGfOGfOGfO-CONH₂ |
| SEQ ID NO: 39 | Alexa Fluor-680-GGG-GfOGfOGfOGfOGfOGfOGfOGfOGfO-CONH₂ |
| SEQ ID NO: 40 | Cyanine 3-GGG-GfOGfOGfOGfOGfOGfOGfOGfOGfO-CONH₂ |
| SEQ ID NO: 41 | Cyanine 5-GGG-GfOGfOGfOGfOGfOGfOGfOGfOGfO-CONH₂ |
| SEQ ID NO: 42 | Cyanine 5.5-GGG-GfOGfOGfOGfOGfOGfOGfOGFOGO-CONH₂ |
| SEQ ID NO: 43 | Cyanine 7-GGG-GfOGfOGfOGfOGfOGfOGfOGfOGfO-CONH₂ |
| SEQ ID NO: 44 | Cyanine 7.5-GGG-GfOGfOGfOGfOGfOGfOGfOGfOGfO-CONH₂ |
| SEQ ID NO: 45 | Cyanine 3-phycoerythrin-GGG-GfOGfOGfOGfOGfOGfOGfOGfOGfO-CONH₂ |
| SEQ ID NO: 46 | Cyanine 5-phycoerythrin-GGG-GfOGfOGfOGfOGfOGfOGfOGfOGfO-CONH₂ |
| SEQ ID NO: 47 | Cyanine 5.5-phycoerythrin-GGG-GfOGfOGfOGfOGfOGfOGfOGfOGfO-CONH₂ |
| SEQ ID NO: 48 | Cyanine 7-phycoerythrin-GGG-GfOGfOGfOGfOGfOGfOGfOGfOGfO-CONH₂ |
| SEQ ID NO: 49 | Cyanine 7.5-phycoerythrin-GGG-GfOGfOGfOGfOGfOGfOGfOGfOGfO-CONH₂ |
| SEQ ID NO: 50 | Rhodamine-GGG-GfOGfOGfOGfOGfOGfOGfOGfOGfO-CONH₂ |
| SEQ ID NO: 51 | Sulfo-Cyanine3-GGG-GfOGfOGfOGfOGfOGfOGfOGfOGfO-CONH₂ |
| SEQ ID NO: 52 | Sulfo-Cyanine5-GGG-GfOGfOGfOGfOGfOGfOGfOGfOGfO-CONH₂ |
| SEQ ID NO: 53 | Sulfo-Cyanine5.5-GGG-GfOGfOGfOGfOGfOGfOGfOGfOGfO-CONH₂ |
| SEQ ID NO: 54 | Sulfo-Cyanine7-GGG-GfOGfOGfOGfOGfOGfOGfOGfOGfO-CONH₂ |
| SEQ ID NO: 55 | Sulfo-Cyanine7.5-GGG-GfOGfOGfOGfOGfOGfOGfOGfOGfO-CONH₂ |
| SEQ ID NO: 56 | Texas Red-GGG-GfOGfOGfOGfOGfOGfOGfOGfOGfO-CONH₂ |
| SEQ ID NO: 57 | Biotin-Ahx-GPOGPOGPOGPOGPOGPOGPOGPOGPO-CONH₂ |
| SEQ ID NO: 58 | 5(6)-Carboxyfluorescein-Ahx-GPOGPOGPOGPOGPOGPOGPOGPOGPO-CONH₂ |
| SEQ ID NO: 59 | Alexa Fluor-350-Ahx-GPOGPOGPOGPOGPOGPOGPOGPOGPO-CONH₂ |
| SEQ ID NO: 60 | Alexa Fluor-430-Ahx-GPOGPOGPOGPOGPOGPOGPOGPOGPO-CONH₂ |
| SEQ ID NO: 61 | Alexa Fluor-488-Ahx-GPOGPOGPOGPOGPOGPOGPOGPOGPO-CONH₂ |
| SEQ ID NO: 62 | Alexa Fluor-546-Ahx-GPOGPOGPOGPOGPOGPOGPOGPOGPO-CONH₂ |
| SEQ ID NO: 63 | Alexa Fluor-568-Ahx-GPOGPOGPOGPOGPOGPOGPOGPOGPO-CONH₂ |
| SEQ ID NO: 64 | Alexa Fluor-594-Ahx-GPOGPOGPOGPOGPOGPOGPOGPOGPO-CONH₂ |
| SEQ ID NO: 65 | Alexa Fluor-633-Ahx-GPOGPOGPOGPOGPOGPOGPOGPOGPO-CONH₂ |
| SEQ ID NO: 66 | Alexa Fluor-660-Ahx-GPOGPOGPOGPOGPOGPOGPOGPOGPO-CONH₂ |
| SEQ ID NO: 67 | Alexa Fluor-680-Ahx-GPOGPOGPOGPOGPOGPOGPOGPOGPO-CONH₂ |
| SEQ ID NO: 68 | Cyanine 3-Ahx-GPOGPOGPOGPOGPOGPOGPOGPOGPO-CONH₂ |
| SEQ ID NO: 69 | Cyanine 5-Ahx-GPOGPOGPOGPOGPOGPOGPOGPOGPO-CONH₂ |
| SEQ ID NO: 70 | Cyanine 5.5-Ahx-GPOGPOGPOGPOGPOGPOGPOGPOGPO-CONH₂ |
| SEQ ID NO: 71 | Cyanine 7-Ahx-GPOGPOGPOGPOGPOGPOGPOGPOGPO-CONH₂ |
| SEQ ID NO: 72 | Cyanine 7.5-Ahx-GPOGPOGPOGPOGPOGPOGPOGPOGPO-CONH₂ |
| SEQ ID NO: 73 | Cyanine 3-phycoerythrin-Ahx-GPOGPOGPOGPOGPOGPOGPOGPOGPO-CONH₂ |
| SEQ ID NO: 74 | Cyanine 5-phycoerythrin-Ahx-GPOGPOGPOGPOGPOGPOGPOGPOGPO-CONH₂ |
| SEQ ID NO: 75 | Cyanine 5.5-phycoerythrin-Ahx-GPOGPOGPOGPOGPOGPOGPOGPOGPO- |
| | CONH₂ |
| SEQ ID NO: 76 | Cyanine 7-phycoerythrin-Ahx-GPOGPOGPOGPOGPOGPOGPOGPOGPO-CONH₂ |
| SEQ ID NO: 77 | Cyanine 7.5-phycoerythrin-Ahx-GPOGPOGPOGPOGPOGPOGPOGPOGPO- |
| | CONH₂ |
| SEQ ID NO: 78 | Rhodamine-Ahx-GPOGPOGPOGPOGPOGPOGPOGPOGPO-CONH2 |
| SEQ ID NO: 79 | Sulfo-Cyanine3-Ahx-GPOGPOGPOGPOGPOGPOGPOGPOGPO-CONH₂ |
| SEQ ID NO: 80 | Sulfo-Cyanine5-Ahx-GPOGPOGPOGPOGPOGPOGPOGPOGPO-CONH₂ |
| SEQ ID NO: 81 | Sulfo-Cyanine5.5-Ahx-GPOGPOGPOGPOGPOGPOGPOGPOGPO-CONH₂ |
| SEQ ID NO: 82 | Sulfo-Cyanine7-Ahx-GPOGPOGPOGPOGPOGPOGPOGPOGPO-CONH₂ |
| SEQ ID NO: 83 | Sulfo-Cyanine7.5-Ahx-GPOGPOGPOGPOGPOGPOGPOGPOGPO-CONH₂ |
| SEQ ID NO: 84 | Texas Red-Ahx-GPOGPOGPOGPOGPOGPOGPOGPOGPO-CONH₂ |
| SEQ ID NO: 85 | Biotin-Ahx-GfOGfOGfOGfOGfOGfOGfOGfOGfO-CONH₂ |
| SEQ ID NO: 86 | 5(6)-Carboxyfluorescein-Ahx-GfOGfOGfOGfOGfOGfOGfOGfOGfO-CONH₂ |
| SEQ ID NO: 87 | Alexa Fluor-350-Ahx-GfOGfOGfOGfOGfOGfOGfOGfOGfO-CONH₂ |
| SEQ ID NO: 88 | Alexa Fluor-430-Ahx-GfOGfOGfOGfOGfOGfOGfOGfOGfO-CONH₂ |
| SEQ ID NO: 89 | Alexa Fluor-488-Ahx-GfOGfOGfOGfOGfOGfOGfOGfOGfO-CONH₂ |
| SEQ ID NO: 90 | Alexa Fluor-546-Ahx-GfOGfOGfOGfOGfOGfOGfOGfOGfO-CONH₂ |
| SEQ ID NO: 91 | Alexa Fluor-568-Ahx-GfOGfOGfOGfOGfOGfOGfOGfOGfO-CONH₂ |
| SEQ ID NO: 92 | Alexa Fluor-594-Ahx-GfOGfOGfOGfOGfOGfOGfOGfOGfO-CONH₂ |
| SEQ ID NO: 93 | Alexa Fluor-633-Ahx-GfOGfOGfOGfOGfOGfOGfOGfOGfO-CONH₂ |
| SEQ ID NO: 94 | Alexa Fluor-660-Ahx-GfOGfOGfOGfOGfOGfOGfOGfOGfO-CONH₂ |
| SEQ ID NO: 95 | Alexa Fluor-680-Ahx-GfOGfOGfOGfOGfOGfOGfOGfOGfO-CONH₂ |
| SEQ ID NO: 96 | Cyanine 3-Ahx-GfOGfOGfOGfOGfOGfOGfOGfOGfO-CONH₂ |
| SEQ ID NO: 97 | Cyanine 5-Ahx-GfOGfOGfOGfOGfOGfOGfOGfOGfO-CONH₂ |
| SEQ ID NO: 98 | Cyanine 5.5-Ahx-GfOGfOGfOGfOGfOGfOGfOGfOGfO-CONH₂ |
| SEQ ID NO: 99 | Cyanine 7-Ahx-GfOGfOGfOGfOGfOGfOGfOGOGO-CONH₂ |
| SEQ ID NO: 100 | Cyanine 7.5-Ahx-GfOGfOGfOGfOGfOGfOGfOGfOGfO-CONH₂ |
| SEQ ID NO: 101 | Cyanine 3-phycoerythrin-Ahx-GfOGfOGfOGfOGfOGfOGfOGfOGfO-CONH₂ |
| SEQ ID NO: 102 | Cyanine 5-phycoerythrin-Ahx-GfOGfOGfOGfOGfOGfOGfOGfOGfO-CONH₂ |
| SEQ ID NO: 103 | Cyanine 5.5-phycoerythrin-Ahx-GfOGfOGfOGfOGfOGfOGfOGfOGfO-CONH₂ |
| SEQ ID NO: 104 | Cyanine 7-phycoerythrin-Ahx-GfOGfOGfOGfOGfOGfOGfOGfOGfO-CONH₂ |
| SEQ ID NO: 105 | Cyanine 7.5-phycoerythrin-Ahx-GfOGfOGfOGfOGfOGfOGfOGfOGfO-CONH₂ |
| SEQ ID NO: 106 | Rhodamine-Ahx-GfOGfOGfOGfOGfOGfOGfOGfOGfO-CONH₂ |
| SEQ ID NO: 107 | Sulfo-Cyanine3-Ahx-GfOGfOGfOGfOGfOGfOGfOGfOGfO-CONH₂ |
| SEQ ID NO: 108 | Sulfo-Cyanine5-Ahx-GfOGfOGfOGfOGfOGfOGfOGfOGfO-CONH₂ |
| SEQ ID NO: 109 | Sulfo-Cyanine5.5-Ahx-GfOGfOGfOGfOGfOGfOGfOGfOGfO-CONH₂ |
| SEQ ID NO: 110 | Sulfo-Cyanine7-Ahx-GfOGfOGfOGfOGfOGfOGfOGfOGfO-CONH₂ |
| SEQ ID NO: 111 | Sulfo-Cyanine7.5-Ahx-GfOGfOGfOGfOGfOGfOGfOGfOGfO-CONH₂ |
| SEQ ID NO: 112 | Texas Red-Ahx-GfOGfOGfOGfOGfOGfOGfOGfOGfO-CONH₂ |
| SEQ ID NO: 113 | Biotin-Ahx-GcOGcOGcOGcOGcOGcOGcOGcOGcO-CONH₂ |
| SEQ ID NO: 114 | 5(6)-Carboxyfluorescein-Ahx-GcOGcOGcOGcOGcOGcOGcOGcOGCO-CONH₂ |
| SEQ ID NO: 115 | Alexa Fluor-350-Ahx-GcOGcOGcOGcOGcOGcOGcOGcOGCO-CONH₂ |
| SEQ ID NO: 116 | Alexa Fluor-430-Ahx-GcOGcOGcOGcOGcOGcOGcOGcOGCO-CONH₂ |
| SEQ ID NO: 117 | Alexa Fluor-488-Ahx-GcOGcOGcOGcOGcOGcOGcOGcOGCO-CONH₂ |
| SEQ ID NO: 118 | Alexa Fluor-546-Ahx-GcOGcOGcOGcOGcOGcOGcOGcOGCO-CONH₂ |
| SEQ ID NO: 119 | Alexa Fluor-568-Ahx-GcOGcOGcOGcOGcOGcOGcOGcOGcO-CONH₂ |
| SEQ ID NO: 120 | Alexa Fluor-594-Ahx-GcOGcOGcOGcOGcOGcOGcOGcOGCO-CONH₂ |
| SEQ ID NO: 121 | Alexa Fluor-633-Ahx-GcOGcOGcOGcOGcOGcOGcOGcOGCO-CONH₂ |
| SEQ ID NO: 122 | Alexa Fluor-660-Ahx-GcOGcOGcOGcOGcOGcOGcOGcOGcO-CONH₂ |
| SEQ ID NO: 123 | Alexa Fluor-680-Ahx-GcOGcOGcOGcOGcOGcOGcOGcOGcO-CONH₂ |
| SEQ ID NO: 124 | Cyanine 3-Ahx-GcOGcOGcOGcOGcOGcOGcOGcOGCO-CONH₂ |
| SEQ ID NO: 125 | Cyanine 5-Ahx-GcOGcOGcOGcOGcOGcOGcOGcOGCO-CONH₂ |
| SEQ ID NO: 126 | Cyanine 5.5-Ahx-GcOGcOGcOGcOGcOGcOGcOGcOGCO-CONH₂ |
| SEQ ID NO: 127 | Cyanine 7-Ahx-GcOGcOGcOGcOGcOGcOGcOGcOGCO-CONH₂ |
| SEQ ID NO: 128 | Cyanine 7.5-Ahx-GcOGcOGcOGcOGcOGcOGcOGcOGCO-CONH₂ |
| SEQ ID NO: 129 | Cyanine 3-phycoerythrin-Ahx-GcOGcOGcOGcOGcOGcOGcOGcOGCO-CONH₂ |
| SEQ ID NO: 130 | Cyanine 5-phycoerythrin-Ahx-GcOGcOGcOGcOGcOGcOGcOGcOGCO-CONH₂ |
| SEQ ID NO: 131 | Cyanine 5.5-phycoerythrin-Ahx-GcOGcOGcOGcOGcOGcOGcOGcOGCO-CONH₂ |
| SEQ ID NO: 132 | Cyanine 7-phycoerythrin-Ahx-GcOGcOGcOGcOGcOGcOGcOGcOGCO-CONH₂ |
| SEQ ID NO: 134 | Cyanine 7.5-phycoerythrin-Ahx-GcOGcOGcOGcOGcOGcOGcOGcOGCO-CONH₂ |
| SEQ ID NO: 135 | Rhodamine-Ahx-GcOGcOGcOGcOGcOGcOGcOGcOGCO-CONH₂ |
| SEQ ID NO: 136 | Sulfo-Cyanine3-Ahx-GcOGcOGcOGcOGcOGcOGcOGcOGCO-CONH₂ |
| SEQ ID NO: 137 | Sulfo-Cyanine5-Ahx-GcOGcOGcOGcOGcOGcOGcOGcOGCO-CONH₂ |
| SEQ ID NO: 138 | Sulfo-Cyanine5.5-Ahx-GcOGcOGcOGcOGcOGcOGcOGcOGCO-CONH₂ |
| SEQ ID NO: 139 | Sulfo-Cyanine7-Ahx-GcOGcOGcOGcOGcOGcOGcOGcOGCO-CONH₂ |
| SEQ ID NO: 140 | Sulfo-Cyanine7.5-Ahx-GcOGcOGcOGcOGcOGcOGcOGcOGCO-CONH₂ |
| SEQ ID NO: 141 | Texas Red-Ahx-GcOGcOGcOGcOGcOGcOGcOGcOGCO-CONH₂ |
| SEQ ID NO: 142 | Biotin-GGG-GcOGcOGcOGcOGcOGcOGcOGcOGCO-CONH₂ |
| SEQ ID NO: 143 | 5(6)-Carboxyfluorescein-GGG-GcOGcOGcOGcOGcOGcOGcOGcOGCO-CONH₂ |
| SEQ ID NO: 144 | Alexa Fluor-350-GGG-GcOGcOGcOGcOGcOGcOGcOGcOGCO-CONH₂ |
| SEQ ID NO: 145 | Alexa Fluor-430-GGG-GcOGcOGcOGcOGcOGcOGcOGcOGCO-CONH₂ |
| SEQ ID NO: 146 | Alexa Fluor-488-GGG-GcOGcOGcOGcOGcOGcOGcOGcOGCO-CONH₂ |
| SEQ ID NO: 147 | Alexa Fluor-546-GGG-GcOGcOGcOGcOGcOGcOGcOGcOGCO-CONH₂ |
| SEQ ID NO: 148 | Alexa Fluor-568-GGG-GcOGcOGcOGcOGcOGcOGcOGcOGCO-CONH₂ |
| SEQ ID NO: 149 | Alexa Fluor-594-GGG-GcOGcOGcOGcOGcOGcOGcOGcOGCO-CONH₂ |
| SEQ ID NO: 150 | Alexa Fluor-633-GGG-GcOGcOGcOGcOGcOGcOGcOGcOGCO-CONH₂ |
| SEQ ID NO: 151 | Alexa Fluor-660-GGG-GcOGcOGcOGcOGcOGcOGcOGcOGCO-CONH₂ |
| SEQ ID NO: 152 | Alexa Fluor-680-GGG-GcOGcOGcOGcOGcOGcOGcOGcOGcO-CONH₂ |
| SEQ ID NO: 153 | Cyanine 3-GGG-GcOGcOGcOGcOGcOGcOGcOGcOGCO-CONH₂ |
| SEQ ID NO: 154 | Cyanine 5-GGG-GcOGcOGcOGcOGcOGcOGcOGcOGCO-CONH₂ |
| SEQ ID NO: 155 | Cyanine 5.5-GGG-GcOGcOGcOGcOGcOGcOGcOGcOGCO-CONH₂ |
| SEQ ID NO: 156 | Cyanine 7-GGG-GcOGcOGcOGcOGcOGcOGcOGcOGCO-CONH₂ |
| SEQ ID NO: 157 | Cyanine 7.5-GGG-GcOGcOGcOGcOGcOGcOGcOGcOGCO-CONH₂ |
| SEQ ID NO: 158 | Cyanine 3-phycoerythrin-GGG-GcOGcOGcOGcOGcOGcOGcOGcOGCO-CONH₂ |
| SEQ ID NO: 159 | Cyanine 5-phycoerythrin-GGG-GcOGcOGcOGcOGcOGcOGcOGcOGCO-CONH₂ |
| SEQ ID NO: 160 | Cyanine 5.5-phycoerythrin-GGG-GcOGcOGcOGcOGcOGcOGcOGcOGcO- |
| | CONH₂ |
| SEQ ID NO: 161 | Cyanine 7-phycoerythrin-GGG-GcOGcOGcOGcOGcOGcOGcOGcOGCO-CONH₂ |
| SEQ ID NO: 162 | Cyanine 7.5-phycoerythrin-GGG-GcOGcOGcOGcOGcOGcOGcOGcOGcO- |
| | CONH₂ |
| SEQ ID NO: 163 | Rhodamine-GGG-GcOGcOGcOGcOGcOGcOGcOGcOGCO-CONH₂ |
| SEQ ID NO: 164 | Sulfo-Cyanine3-GGG-GcOGcOGcOGcOGcOGcOGcOGcOGCO-CONH₂ |
| SEQ ID NO: 165 | Sulfo-Cyanine5-GGG-GcOGcOGcOGcOGcOGcOGcOGcOGCO-CONH₂ |
| SEQ ID NO: 166 | Sulfo-Cyanine5.5-GGG-GcOGcOGcOGcOGcOGcOGcOGcOGCO-CONH₂ |
| SEQ ID NO: 167 | Sulfo-Cyanine7-GGG-GcOGcOGcOGcOGcOGcOGcOGcOGCO-CONH₂ |
| SEQ ID NO: 168 | Sulfo-Cyanine7.5-GGG-GcOGcOGcOGcOGcOGcOGcOGcOGCO-CONH₂ |
| SEQ ID NO: 169 | Texas Red-GGG-GcOGcOGcOGcOGcOGcOGcOGcOGCO-CONH₂ |
| SEQ ID NO: 170 | Biotin-G3-(GPO)3-GPLGLAGIG-(GPO)₂-CONH₂ |
| SEQ ID NO: 171 | 5(6)-Carboxyfluorescein-G3-(GPO)3-GPLGLAGIG-(GPO)₂-CONH₂ |
| SEQ ID NO: 172 | Alexa Fluor-350-G3-(GPO)3-GPLGLAGIG-(GPO)2-CONH₂ |
| SEQ ID NO: 173 | Alexa Fluor-430-G3-(GPO)3-GPLGLAGIG-(GPO)2-CONH₂ |
| SEQ ID NO: 174 | Alexa Fluor-488-G3-(GPO)3-GPLGLAGIG-(GPO)2-CONH₂ |
| SEQ ID NO: 175 | Alexa Fluor-546-G3-(GPO)3-GPLGLAGIG-(GPO)2-CONH₂ |
| SEQ ID NO: 176 | Alexa Fluor-568-G3-(GPO)3-GPLGLAGIG-(GPO)2-CONH₂ |
| SEQ ID NO: 177 | Alexa Fluor-594-G3-(GPO)3-GPLGLAGIG-(GPO)2-CONH₂ |
| SEQ ID NO: 178 | Alexa Fluor-633-G3-(GPO)3-GPLGLAGIG-(GPO)2-CONH₂ |
| SEQ ID NO: 179 | Alexa Fluor-660-G3-(GPO)3-GPLGLAGIG-(GPO)2-CONH₂ |
| SEQ ID NO: 180 | Alexa Fluor-680-G3-(GPO)3-GPLGLAGIG-(GPO)2-CONH₂ |
| SEQ ID NO: 181 | Cyanine 3-G3-(GPO)3-GPLGLAGIG-(GPO)2-CONH₂ |
| SEQ ID NO: 182 | Cyanine 5-G3-(GPO)3-GPLGLAGIG-(GPO)2-CONH₂ |
| SEQ ID NO: 183 | Cyanine 5.5-G3-(GPO)3-GPLGLAGIG-(GPO)2-CONH₂ |
| SEQ ID NO: 184 | Cyanine 7-G3-(GPO)3-GPLGLAGIG-(GPO)2-CONH₂ |
| SEQ ID NO: 185 | Cyanine 7.5-G3-(GPO)3-GPLGLAGIG-(GPO)2-CONH₂ |
| SEQ ID NO: 186 | Cyanine 3-phycoerythrin-G3-(GPO)3-GPLGLAGIG-(GPO)2-CONH₂ |
| SEQ ID NO: 187 | Cyanine 5-phycoerythrin-G3-(GPO)3-GPLGLAGIG-(GPO)2-CONH₂ |
| SEQ ID NO: 188 | Cyanine 5.5-phycoerythrin-G3-(GPO)3-GPLGLAGIG-(GPO)2-CONH₂ |
| SEQ ID NO: 189 | Cyanine 7-phycoerythrin-G3-(GPO)3-GPLGLAGIG-(GPO)2-CONH₂ |
| SEQ ID NO: 190 | Cyanine 7.5-phycoerythrin-G3-(GPO)3-GPLGLAGIG-(GPO)2-CONH₂ |
| SEQ ID NO: 191 | Rhodamine-G3-(GPO)3-GPLGLAGIG-(GPO)2-CONH₂ |
| SEQ ID NO: 192 | Sulfo-Cyanine3-G3-(GPO)3-GPLGLAGIG-(GPO)2-CONH₂ |
| SEQ ID NO: 193 | Sulfo-Cyanine5-G3-(GPO)3-GPLGLAGIG-(GPO)2-CONH₂ |
| SEQ ID NO: 194 | Sulfo-Cyanine5.5-G3-(GPO)3-GPLGLAGIG-(GPO)2-CONH₂ |
| SEQ ID NO: 195 | Sulfo-Cyanine7-G3-(GPO)3-GPLGLAGIG-(GPO)2-CONH₂ |
| SEQ ID NO: 196 | Sulfo-Cyanine7.5-G3-(GPO)3-GPLGLAGIG-(GPO)2-CONH₂ |
| SEQ ID NO: 197 | Texas Red-G3-(GPO)3-GPLGLAGIG-(GPO)2-CONH₂ |
| SEQ ID NO: 198 | Biotin-G3-(GPO)3-GPLGLAGIG-(GPO)3-CONH₂ |
| SEQ ID NO: 199 | 5(6)-Carboxyfluorescein-G3-(GPO)3-GPLGLAGIG-(GPO)3-CONH₂ |
| SEQ ID NO: 200 | Alexa Fluor-350-G3-(GPO)3-GPLGLAGIG-(GPO)3-CONH₂ |
| SEQ ID NO: 201 | Alexa Fluor-430-G3-(GPO)3-GPLGLAGIG-(GPO)3-CONH₂ |
| SEQ ID NO: 202 | Alexa Fluor-488-G3-(GPO)3-GPLGLAGIG-(GPO)3-CONH₂ |
| SEQ ID NO: 203 | Alexa Fluor-546-G3-(GPO)3-GPLGLAGIG-(GPO)3-CONH₂ |
| SEQ ID NO: 204 | Alexa Fluor-568-G3-(GPO)3-GPLGLAGIG-(GPO)3-CONH₂ |
| SEQ ID NO: 205 | Alexa Fluor-594-G3-(GPO)3-GPLGLAGIG-(GPO)3-CONH₂ |
| SEQ ID NO: 206 | Alexa Fluor-633-G3-(GPO)3-GPLGLAGIG-(GPO)3-CONH₂ |
| SEQ ID NO: 207 | Alexa Fluor-660-G3-(GPO)3-GPLGLAGIG-(GPO)3-CONH₂ |
| SEQ ID NO: 208 | Alexa Fluor-680-G3-(GPO)3-GPLGLAGIG-(GPO)3-CONH₂ |
| SEQ ID NO: 209 | Cyanine 3-G3-(GPO)3-GPLGLAGIG-(GPO)3-CONH₂ |
| SEQ ID NO: 210 | Cyanine 5-G3-(GPO)3-GPLGLAGIG-(GPO)3-CONH₂ |
| SEQ ID NO: 211 | Cyanine 5.5-G3-(GPO)3-GPLGLAGIG-(GPO)3-CONH₂ |
| SEQ ID NO: 212 | Cyanine 7-G3-(GPO)3-GPLGLAGIG-(GPO)3-CONH₂ |
| SEQ ID NO: 213 | Cyanine 7.5-G3-(GPO)3-GPLGLAGIG-(GPO)3-CONH₂ |
| SEQ ID NO: 214 | Cyanine 3-phycoerythrin-G3-(GPO)3-GPLGLAGIG-(GPO)3-CONH₂ |
| SEQ ID NO: 215 | Cyanine 5-phycoerythrin-G3-(GPO)3-GPLGLAGIG-(GPO)3-CONH₂ |
| SEQ ID NO: 216 | Cyanine 5.5-phycoerythrin-G3-(GPO)3-GPLGLAGIG-(GPO)3-CONH₂ |
| SEQ ID NO: 217 | Cyanine 7-phycoerythrin-G3-(GPO)3-GPLGLAGIG-(GPO)3-CONH₂ |
| SEQ ID NO: 218 | Cyanine 7.5-phycoerythrin-G3-(GPO)3-GPLGLAGIG-(GPO)3-CONH₂ |
| SEQ ID NO: 219 | Rhodamine-G3-(GPO)3-GPLGLAGIG-(GPO)3-CONH₂ |
| SEQ ID NO: 220 | Sulfo-Cyanine3-G3-(GPO)3-GPLGLAGIG-(GPO)3-CONH₂ |
| SEQ ID NO: 221 | Sulfo-Cyanine5-G3-(GPO)3-GPLGLAGIG-(GPO)3-CONH₂ |
| SEQ ID NO: 222 | Sulfo-Cyanine5.5-G3-(GPO)3-GPLGLAGIG-(GPO)3-CONH₂ |
| SEQ ID NO: 223 | Sulfo-Cyanine7-G3-(GPO)3-GPLGLAGIG-(GPO)3-CONH₂ |
| SEQ ID NO: 224 | Sulfo-Cyanine7.5-G3-(GPO)3-GPLGLAGIG-(GPO)3-CONH₂ |
| SEQ ID NO: 225 | Texas Red-G3-(GPO)3-GPLGLAGIG-(GPO)3-CONH₂ |
| SEQ ID NO: 226 | Biotin-G3-(GfO)3-GPLGLAGIG-(GPO)3-CONH₂ |
| SEQ ID NO: 227 | 5(6)-Carboxyfluorescein-G3-(GfO)3-GPLGLAGIG-(GPO)3-CONH₂ |
| SEQ ID NO: 228 | Alexa Fluor-350-G3-(GfO)3-GPLGLAGIG-(GPO)3-CONH₂ |
| SEQ ID NO: 229 | Alexa Fluor-430-G3-(GfO)3-GPLGLAGIG-(GPO)3-CONH₂ |
| SEQ ID NO: 230 | Alexa Fluor-488-G3-(GfO)3-GPLGLAGIG-(GPO)3-CONH₂ |
| SEQ ID NO: 231 | Alexa Fluor-546-G3-(GfO)3-GPLGLAGIG-(GPO)3-CONH₂ |
| SEQ ID NO: 232 | Alexa Fluor-568-G3-(GfO)3-GPLGLAGIG-(GPO)3-CONH₂ |
| SEQ ID NO: 233 | Alexa Fluor-594-G3-(GfO)3-GPLGLAGIG-(GPO)3-CONH₂ |
| SEQ ID NO: 234 | Alexa Fluor-633-G3-(GfO)3-GPLGLAGIG-(GPO)3-CONH₂ |
| SEQ ID NO: 235 | Alexa Fluor-660-G3-(GfO)3-GPLGLAGIG-(GPO)3-CONH₂ |
| SEQ ID NO: 236 | Alexa Fluor-680-G3-(GfO)3-GPLGLAGIG-(GPO)3-CONH₂ |
| SEQ ID NO: 237 | Cyanine 3-G3-(GfO)3-GPLGLAGIG-(GPO)3-CONH₂ |
| SEQ ID NO: 238 | Cyanine 5-G3-(GfO)3-GPLGLAGIG-(GPO)3-CONH₂ |
| SEQ ID NO: 239 | Cyanine 5.5-G3-(GfO)3-GPLGLAGIG-(GPO)3-CONH₂ |
| SEQ ID NO: 240 | Cyanine 7-G3-(GfO)3-GPLGLAGIG-(GPO)3-CONH₂ |
| SEQ ID NO: 241 | Cyanine 7.5-G3-(GfO)3-GPLGLAGIG-(GPO)3-CONH₂ |
| SEQ ID NO: 242 | Cyanine 3-phycoerythrin-G3-(GfO)3-GPLGLAGIG-(GPO)3-CONH₂ |
| SEQ ID NO: 243 | Cyanine 5-phycoerythrin-G3-(GfO)3-GPLGLAGIG-(GPO)3-CONH₂ |
| SEQ ID NO: 244 | Cyanine 5.5-phycoerythrin-G3-(GfO)3-GPLGLAGIG-(GPO)3-CONH₂ |
| SEQ ID NO: 245 | Cyanine 7-phycoerythrin-G3-(GfO)3-GPLGLAGIG-(GPO)3-CONH₂ |
| SEQ ID NO: 246 | Cyanine 7.5-phycoerythrin-G3-(GfO)3-GPLGLAGIG-(GPO)3-CONH₂ |
| SEQ ID NO: 247 | Rhodamine-G3-(GfO)3-GPLGLAGIG-(GPO)3-CONH₂ |
| SEQ ID NO: 248 | Sulfo-Cyanine3-G3-(GfO)3-GPLGLAGIG-(GPO)3-CONH₂ |
| SEQ ID NO: 249 | Sulfo-Cyanine5-G3-(GfO)3-GPLGLAGIG-(GPO)3-CONH₂ |
| SEQ ID NO: 250 | Sulfo-Cyanine5.5-G3-(GfO)3-GPLGLAGIG-(GPO)3-CONH₂ |
| SEQ ID NO: 251 | Sulfo-Cyanine7-G3-(GfO)3-GPLGLAGIG-(GPO)3-CONH₂ |
| SEQ ID NO: 252 | Sulfo-Cyanine7.5-G3-(GfO)3-GPLGLAGIG-(GPO)3-CONH₂ |
| SEQ ID NO: 253 | Texas Red-G3-(GfO)3-GPLGLAGIG-(GPO)3-CONH₂ |
| SEQ ID NO: 254 | Biotin-G3-(GfO)3-GPLGLAGIG-(GPO)₂-CONH₂ |
| SEQ ID NO: 255 | 5(6)-Carboxyfluorescein-G3-(GfO)3-GPLGLAGIG-(GPO)₂-CONH₂ |
| SEQ ID NO: 256 | Alexa Fluor-350-G3-(GfO)3-GPLGLAGIG-(GPO)2-CONH₂ |
| SEQ ID NO: 257 | Alexa Fluor-430-G3-(GfO)3-GPLGLAGIG-(GPO)2-CONH₂ |
| SEQ ID NO: 258 | Alexa Fluor-488-G3-(GfO)3-GPLGLAGIG-(GPO)2-CONH₂ |
| SEQ ID NO: 259 | Alexa Fluor-546-G3-(GfO)3-GPLGLAGIG-(GPO)2-CONH₂ |
| SEQ ID NO: 260 | Alexa Fluor-568-G3-(GfO)3-GPLGLAGIG-(GPO)2-CONH₂ |
| SEQ ID NO: 261 | Alexa Fluor-594-G3-(GfO)3-GPLGLAGIG-(GPO)2-CONH₂ |
| SEQ ID NO: 262 | Alexa Fluor-633-G3-(GfO)3-GPLGLAGIG-(GPO)2-CONH₂ |
| SEQ ID NO: 263 | Alexa Fluor-660-G3-(GfO)3-GPLGLAGIG-(GPO)2-CONH₂ |
| SEQ ID NO: 264 | Alexa Fluor-680-G3-(GfO)3-GPLGLAGIG-(GPO)2-CONH₂ |
| SEQ ID NO: 265 | Cyanine 3-G3-(GfO)3-GPLGLAGIG-(GPO)2-CONH₂ |
| SEQ ID NO: 266 | Cyanine 5-G3-(GfO)3-GPLGLAGIG-(GPO)2-CONH₂ |
| SEQ ID NO: 267 | Cyanine 5.5-G3-(GfO)3-GPLGLAGIG-(GPO)2-CONH₂ |
| SEQ ID NO: 268 | Cyanine 7-G3-(GfO)3-GPLGLAGIG-(GPO)2-CONH₂ |
| SEQ ID NO: 269 | Cyanine 7.5-G3-(GfO)3-GPLGLAGIG-(GPO)2-CONH₂ |
| SEQ ID NO: 270 | Cyanine 3-phycoerythrin-G3-(GfO)3-GPLGLAGIG-(GPO)2-CONH₂ |
| SEQ ID NO: 271 | Cyanine 5-phycoerythrin-G3-(GfO)3-GPLGLAGIG-(GPO)2-CONH₂ |
| SEQ ID NO: 272 | Cyanine 5.5-phycoerythrin-G3-(GfO)3-GPLGLAGIG-(GPO)2-CONH₂ |
| SEQ ID NO: 273 | Cyanine 7-phycoerythrin-G3-(GfO)3-GPLGLAGIG-(GPO)2-CONH₂ |
| SEQ ID NO: 274 | Cyanine 7.5-phycoerythrin-G3-(GfO)3-GPLGLAGIG-(GPO)2-CONH₂ |
| SEQ ID NO: 275 | Rhodamine-G3-(GfO)3-GPLGLAGIG-(GPO)2-CONH₂ |
| SEQ ID NO: 276 | Sulfo-Cyanine3-G3-(GfO)3-GPLGLAGIG-(GPO)2-CONH₂ |
| SEQ ID NO: 277 | Sulfo-Cyanine5-G3-(GfO)3-GPLGLAGIG-(GPO)2-CONH₂ |
| SEQ ID NO: 278 | Sulfo-Cyanine5.5-G3-(GfO)3-GPLGLAGIG-(GPO)2-CONH₂ |
| SEQ ID NO: 279 | Sulfo-Cyanine7-G3-(GfO)3-GPLGLAGIG-(GPO)2-CONH₂ |
| SEQ ID NO: 280 | Sulfo-Cyanine7.5-G3-(GfO)3-GPLGLAGIG-(GPO)2-CONH₂ |
| SEQ ID NO: 281 | Texas Red-G3-(GfO)3-GPLGLAGIG-(GPO)2-CONH₂ |
| SEQ ID NO: 282 | Biotin-G3-(GfO)3-GPLGLAGIG-(GfO)₂-CONH₂ |
| SEQ ID NO: 283 | 5(6)-Carboxyfluorescein-G3-(GfO)3-GPLGLAGIG-(GfO)2-CONH₂ |
| SEQ ID NO: 284 | Alexa Fluor-350-G3-(GfO)3-GPLGLAGIG-(GfO)2-CONH₂ |
| SEQ ID NO: 285 | Alexa Fluor-430-G3-(GfO)3-GPLGLAGIG-(GfO)2-CONH₂ |
| SEQ ID NO: 286 | Alexa Fluor-488-G3-(GfO)3-GPLGLAGIG-(GfO)2-CONH₂ |
| SEQ ID NO: 287 | Alexa Fluor-546-G3-(GfO)3-GPLGLAGIG-(GfO)2-CONH₂ |
| SEQ ID NO: 288 | Alexa Fluor-568-G3-(GfO)3-GPLGLAGIG-(GfO)2-CONH₂ |
| SEQ ID NO: 289 | Alexa Fluor-594-G3-(GfO)3-GPLGLAGIG-(GfO)2-CONH₂ |
| SEQ ID NO: 290 | Alexa Fluor-633-G3-(GfO)3-GPLGLAGIG-(GfO)2-CONH₂ |
| SEQ ID NO: 291 | Alexa Fluor-660-G3-(GfO)3-GPLGLAGIG-(GfO)2-CONH₂ |
| SEQ ID NO: 292 | Alexa Fluor-680-G3-(GfO)3-GPLGLAGIG-(GfO)2-CONH₂ |
| SEQ ID NO: 293 | Cyanine 3-G3-(GfO)3-GPLGLAGIG-(GfO)2-CONH₂ |
| SEQ ID NO: 294 | Cyanine 5-G3-(GfO)3-GPLGLAGIG-(GfO)2-CONH₂ |
| SEQ ID NO: 295 | Cyanine 5.5-G3-(GfO)3-GPLGLAGIG-(GfO)2-CONH₂ |
| SEQ ID NO: 296 | Cyanine 7-G3-(GfO)3-GPLGLAGIG-(GfO)2-CONH₂ |
| SEQ ID NO: 297 | Cyanine 7.5-G3-(GfO)3-GPLGLAGIG-(GfO)2-CONH₂ |
| SEQ ID NO: 298 | Cyanine 3-phycoerythrin-G3-(GfO)3-GPLGLAGIG-(GfO)2-CONH₂ |
| SEQ ID NO: 299 | Cyanine 5-phycoerythrin-G3-(GfO)3-GPLGLAGIG-(GfO)2-CONH₂ |
| SEQ ID NO: 300 | Cyanine 5.5-phycoerythrin-G3-(GfO)3-GPLGLAGIG-(GfO)2-CONH₂ |
| SEQ ID NO: 301 | Cyanine 7-phycoerythrin-G3-(GfO)3-GPLGLAGIG-(GfO)2-CONH₂ |
| SEQ ID NO: 302 | Cyanine 7.5-phycoerythrin-G3-(GfO)3-GPLGLAGIG-(GfO)2-CONH₂ |
| SEQ ID NO: 303 | Rhodamine-G3-(GfO)3-GPLGLAGIG-(GfO)2-CONH₂ |
| SEQ ID NO: 304 | Sulfo-Cyanine3-G3-(GfO)3-GPLGLAGIG-(GfO)2-CONH₂ |
| SEQ ID NO: 305 | Sulfo-Cyanine5-G3-(GfO)3-GPLGLAGIG-(GfO)2-CONH₂ |
| SEQ ID NO: 306 | Sulfo-Cyanine5.5-G3-(GfO)3-GPLGLAGIG-(GfO)2-CONH₂ |
| SEQ ID NO: 307 | Sulfo-Cyanine7-G3-(GfO)3-GPLGLAGIG-(GfO)2-CONH₂ |
| SEQ ID NO: 308 | Sulfo-Cyanine7.5-G3-(GfO)3-GPLGLAGIG-(GfO)2-CONH₂ |
| SEQ ID NO: 309 | Texas Red-G3-(GfO)3-GPLGLAGIG-(GfO)2-CONH₂ |
| SEQ ID NO: 310 | Biotin-G3-(GfO)3-GPLGLAGIG-(GfO)3-CONH₂ |
| SEQ ID NO: 311 | 5(6)-Carboxyfluorescein-G3-(GfO)3-GPLGLAGIG-(GfO)3-CONH₂ |
| SEQ ID NO: 312 | Alexa Fluor-350-G3-(GfO)3-GPLGLAGIG-(GfO)3-CONH₂ |
| SEQ ID NO: 313 | Alexa Fluor-430-G3-(GfO)3-GPLGLAGIG-(GfO)3-CONH₂ |
| SEQ ID NO: 314 | Alexa Fluor-488-G3-(GfO)3-GPLGLAGIG-(GfO)3-CONH₂ |
| SEQ ID NO: 315 | Alexa Fluor-546-G3-(GfO)3-GPLGLAGIG-(GfO)3-CONH₂ |
| SEQ ID NO: 316 | Alexa Fluor-568-G3-(GfO)3-GPLGLAGIG-(GfO)3-CONH₂ |
| SEQ ID NO: 317 | Alexa Fluor-594-G3-(GfO)3-GPLGLAGIG-(GfO)3-CONH₂ |
| SEQ ID NO: 318 | Alexa Fluor-633-G3-(GfO)3-GPLGLAGIG-(GfO)3-CONH₂ |
| SEQ ID NO: 319 | Alexa Fluor-660-G3-(GfO)3-GPLGLAGIG-(GfO)3-CONH₂ |
| SEQ ID NO: 320 | Alexa Fluor-680-G3-(GfO)3-GPLGLAGIG-(GfO)3-CONH₂ |
| SEQ ID NO: 321 | Cyanine 3-G3-(GfO)3-GPLGLAGIG-(GfO)3-CONH₂ |
| SEQ ID NO: 322 | Cyanine 5-G3-(GfO)3-GPLGLAGIG-(GfO)3-CONH₂ |
| SEQ ID NO: 323 | Cyanine 5.5-G3-(GfO)3-GPLGLAGIG-(GfO)3-CONH₂ |
| SEQ ID NO: 324 | Cyanine 7-G3-(GfO)3-GPLGLAGIG-(GfO)3-CONH₂ |
| SEQ ID NO: 325 | Cyanine 7.5-G3-(GfO)3-GPLGLAGIG-(GfO)3-CONH₂ |
| SEQ ID NO: 326 | Cyanine 3-phycoerythrin-G3-(GfO)3-GPLGLAGIG-(GfO)3-CONH₂ |
| SEQ ID NO: 327 | Cyanine 5-phycoerythrin-G3-(GfO)3-GPLGLAGIG-(GfO)3-CONH₂ |
| SEQ ID NO: 328 | Cyanine 5.5-phycoerythrin-G3-(GfO)3-GPLGLAGIG-(GfO)3-CONH₂ |
| SEQ ID NO: 329 | Cyanine 7-phycoerythrin-G3-(GfO)3-GPLGLAGIG-(GfO)3-CONH₂ |
| SEQ ID NO: 330 | Cyanine 7.5-phycoerythrin-G3-(GfO)3-GPLGLAGIG-(GfO)3-CONH₂ |
| SEQ ID NO: 331 | Rhodamine-G3-(GfO)3-GPLGLAGIG-(GfO)3-CONH₂ |
| SEQ ID NO: 332 | Sulfo-Cyanine3-G3-(GfO)3-GPLGLAGIG-(GfO)3-CONH₂ |
| SEQ ID NO: 333 | Sulfo-Cyanine5-G3-(GfO)3-GPLGLAGIG-(GfO)3-CONH₂ |
| SEQ ID NO: 334 | Sulfo-Cyanine5.5-G3-(GfO)3-GPLGLAGIG-(GfO)3-CONH₂ |
| SEQ ID NO: 335 | Sulfo-Cyanine7-G3-(GfO)3-GPLGLAGIG-(GfO)3-CONH₂ |
| SEQ ID NO: 336 | Sulfo-Cyanine7.5-G3-(GfO)3-GPLGLAGIG-(GfO)3-CONH₂ |
| SEQ ID NO: 337 | Texas Red-G3-(GfO)3-GPLGLAGIG-(GfO)3-CONH₂ |
| SEQ ID NO: 338 | Biotin-G3-(GPO)3-GPQGIAGQG-(GPO)2-CONH₂ |
| SEQ ID NO: 339 | 5(6)-Carboxyfluorescein-G3-(GPO)3-GPQGIAGQG-(GPO)₂-CONH₂ |
| SEQ ID NO: 340 | Alexa Fluor-350-G3-(GPO)3-GPQGIAGQG-(GPO)2-CONH₂ |
| SEQ ID NO: 341 | Alexa Fluor-430-G3-(GPO)3-GPQGIAGQG-(GPO)2-CONH₂ |
| SEQ ID NO: 342 | Alexa Fluor-488-G3-(GPO)3-GPQGIAGQG-(GPO)2-CONH₂ |
| SEQ ID NO: 343 | Alexa Fluor-546-G3-(GPO)3-GPQGIAGQG-(GPO)2-CONH₂ |
| SEQ ID NO: 344 | Alexa Fluor-568-G3-(GPO)3-GPQGIAGQG-(GPO)2-CONH₂ |
| SEQ ID NO: 345 | Alexa Fluor-594-G3-(GPO)3-GPQGIAGQG-(GPO)2-CONH₂ |
| SEQ ID NO: 346 | Alexa Fluor-633-G3-(GPO)3-GPQGIAGQG-(GPO)2-CONH₂ |
| SEQ ID NO: 347 | Alexa Fluor-660-G3-(GPO)3-GPQGIAGQG-(GPO)2-CONH₂ |
| SEQ ID NO: 348 | Alexa Fluor-680-G3-(GPO)3-GPQGIAGQG-(GPO)2-CONH₂ |
| SEQ ID NO: 349 | Cyanine 3-G3-(GPO)3-GPQGIAGQG-(GPO)2-CONH₂ |
| SEQ ID NO: 350 | Cyanine 5-G3-(GPO)3-GPQGIAGQG-(GPO)2-CONH₂ |
| SEQ ID NO: 351 | Cyanine 5.5-G3-(GPO)3-GPQGIAGQG-(GPO)2-CONH₂ |
| SEQ ID NO: 352 | Cyanine 7-G3-(GPO)3-GPQGIAGQG-(GPO)2-CONH₂ |
| SEQ ID NO: 353 | Cyanine 7.5-G3-(GPO)3-GPQGIAGQG-(GPO)2-CONH₂ |
| SEQ ID NO: 354 | Cyanine 3-phycoerythrin-G3-(GPO)3-GPQGIAGQG-(GPO)2-CONH₂ |
| SEQ ID NO: 355 | Cyanine 5-phycoerythrin-G3-(GPO)3-GPQGIAGQG-(GPO)2-CONH₂ |
| SEQ ID NO: 356 | Cyanine 5.5-phycoerythrin-G3-(GPO)3-GPQGIAGQG-(GPO)2-CONH₂ |
| SEQ ID NO: 357 | Cyanine 7-phycoerythrin-G3-(GPO)3-GPQGIAGQG-(GPO)2-CONH₂ |
| SEQ ID NO: 358 | Cyanine 7.5-phycoerythrin-G3-(GPO)3-GPQGIAGQG-(GPO)2-CONH₂ |
| SEQ ID NO: 359 | Rhodamine-G3-(GPO)3-GPQGIAGQG-(GPO)2-CONH₂ |
| SEQ ID NO: 360 | Sulfo-Cyanine3-G3-(GPO)3-GPQGIAGQG-(GPO)2-CONH₂ |
| SEQ ID NO: 361 | Sulfo-Cyanine5-G3-(GPO)3-GPQGIAGQG-(GPO)2-CONH₂ |
| SEQ ID NO: 362 | Sulfo-Cyanine5.5-G3-(GPO)3-GPQGIAGQG-(GPO)2-CONH₂ |
| SEQ ID NO: 363 | Sulfo-Cyanine7-G3-(GPO)3-GPQGIAGQG-(GPO)2-CONH₂ |
| SEQ ID NO: 364 | Sulfo-Cyanine7.5-G3-(GPO)3-GPQGIAGQG-(GPO)2-CONH₂ |
| SEQ ID NO: 365 | Texas Red-G3-(GPO)3-GPQGIAGQG-(GPO)2-CONH₂ |
| SEQ ID NO: 366 | Biotin-G3-(GPO)3-GPQGIAGQG-(GPO)3-CONH₂ |
| SEQ ID NO: 367 | 5(6)-Carboxyfluorescein-G3-(GPO)3-GPQGIAGQG-(GPO)3-CONH₂ |
| SEQ ID NO: 368 | Alexa Fluor-350-G3-(GPO)3-GPQGIAGQG-(GPO)3-CONH₂ |
| SEQ ID NO: 369 | Alexa Fluor-430-G3-(GPO)3-GPQGIAGQG-(GPO)3-CONH₂ |
| SEQ ID NO: 370 | Alexa Fluor-488-G3-(GPO)3-GPQGIAGQG-(GPO)3-CONH₂ |
| SEQ ID NO: 371 | Alexa Fluor-546-G3-(GPO)3-GPQGIAGQG-(GPO)3-CONH₂ |
| SEQ ID NO: 372 | Alexa Fluor-568-G3-(GPO)3-GPQGIAGQG-(GPO)3-CONH₂ |
| SEQ ID NO: 373 | Alexa Fluor-594-G3-(GPO)3-GPQGIAGQG-(GPO)3-CONH₂ |
| SEQ ID NO: 374 | Alexa Fluor-633-G3-(GPO)3-GPQGIAGQG-(GPO)3-CONH₂ |
| SEQ ID NO: 375 | Alexa Fluor-660-G3-(GPO)3-GPQGIAGQG-(GPO)3-CONH₂ |
| SEQ ID NO: 376 | Alexa Fluor-680-G3-(GPO)3-GPQGIAGQG-(GPO)3-CONH₂ |
| SEQ ID NO: 377 | Cyanine 3-G3-(GPO)3-GPQGIAGQG-(GPO)3-CONH₂ |
| SEQ ID NO: 378 | Cyanine 5-G3-(GPO)3-GPQGIAGQG-(GPO)3-CONH₂ |
| SEQ ID NO: 379 | Cyanine 5.5-G3-(GPO)3-GPQGIAGQG-(GPO)3-CONH₂ |
| SEQ ID NO: 380 | Cyanine 7-G3-(GPO)3-GPQGIAGQG-(GPO)3-CONH₂ |
| SEQ ID NO: 381 | Cyanine 7.5-G3-(GPO)3-GPQGIAGQG-(GPO)3-CONH₂ |
| SEQ ID NO: 382 | Cyanine 3-phycoerythrin-G3-(GPO)3-GPQGIAGQG-(GPO)3-CONH₂ |
| SEQ ID NO: 383 | Cyanine 5-phycoerythrin-G3-(GPO)3-GPQGIAGQG-(GPO)3-CONH₂ |
| SEQ ID NO: 384 | Cyanine 5.5-phycoerythrin-G3-(GPO)3-GPQGIAGQG-(GPO)3-CONH₂ |
| SEQ ID NO: 385 | Cyanine 7-phycoerythrin-G3-(GPO)3-GPQGIAGQG-(GPO)3-CONH₂ |
| SEQ ID NO: 386 | Cyanine 7.5-phycoerythrin-G3-(GPO)3-GPQGIAGQG-(GPO)3-CONH₂ |
| SEQ ID NO: 387 | Rhodamine-G3-(GPO)3-GPQGIAGQG-(GPO)3-CONH₂ |
| SEQ ID NO: 388 | Sulfo-Cyanine3-G3-(GPO)3-GPQGIAGQG-(GPO)3-CONH₂ |
| SEQ ID NO: 389 | Sulfo-Cyanine5-G3-(GPO)3-GPQGIAGQG-(GPO)3-CONH₂ |
| SEQ ID NO: 390 | Sulfo-Cyanine5.5-G3-(GPO)3-GPQGIAGQG-(GPO)3-CONH₂ |
| SEQ ID NO: 391 | Sulfo-Cyanine7-G3-(GPO)3-GPQGIAGQG-(GPO)3-CONH₂ |
| SEQ ID NO: 392 | Sulfo-Cyanine7.5-G3-(GPO)3-GPQGIAGQG-(GPO)3-CONH₂ |
| SEQ ID NO: 393 | Texas Red-G3-(GPO)3-GPQGIAGQG-(GPO)3-CONH₂ |
| SEQ ID NO: 394 | Biotin-G3-(GfO)3-GPQGIAGQG-(GPO)3-CONH₂ |
| SEQ ID NO: 395 | 5(6)-Carboxyfluorescein-G3-(GfO)3-GPQGIAGQG-(GPO)3-CONH₂ |
| SEQ ID NO: 396 | Alexa Fluor-350-G3-(GfO)3-GPQGIAGQG-(GPO)3-CONH₂ |
| SEQ ID NO: 397 | Alexa Fluor-430-G3-(GfO)3-GPQGIAGQG-(GPO)3-CONH₂ |
| SEQ ID NO: 398 | Alexa Fluor-488-G3-(GfO)3-GPQGIAGQG-(GPO)3-CONH₂ |
| SEQ ID NO: 399 | Alexa Fluor-546-G3-(GfO)3-GPQGIAGQG-(GPO)3-CONH₂ |
| SEQ ID NO: 400 | Alexa Fluor-568-G3-(GfO)3-GPQGIAGQG-(GPO)3-CONH₂ |
| SEQ ID NO: 401 | Alexa Fluor-594-G3-(GfO)3-GPQGIAGQG-(GPO)3-CONH₂ |
| SEQ ID NO: 402 | Alexa Fluor-633-G3-(GfO)3-GPQGIAGQG-(GPO)3-CONH₂ |
| SEQ ID NO: 403 | Alexa Fluor-660-G3-(GfO)3-GPQGIAGQG-(GPO)3-CONH₂ |
| SEQ ID NO: 404 | Alexa Fluor-680-G3-(GfO)3-GPQGIAGQG-(GPO)3-CONH₂ |
| SEQ ID NO: 405 | Cyanine 3-G3-(GfO)3-GPQGIAGQG-(GPO)3-CONH₂ |
| SEQ ID NO: 406 | Cyanine 5-G3-(GfO)3-GPQGIAGQG-(GPO)3-CONH₂ |
| SEQ ID NO: 407 | Cyanine 5.5-G3-(GfO)3-GPQGIAGQG-(GPO)3-CONH₂ |
| SEQ ID NO: 408 | Cyanine 7-G3-(GfO)3-GPQGIAGQG-(GPO)3-CONH₂ |
| SEQ ID NO: 409 | Cyanine 7.5-G3-(GfO)3-GPQGIAGQG-(GPO)3-CONH₂ |
| SEQ ID NO: 410 | Cyanine 3-phycoerythrin-G3-(GfO)3-GPQGIAGQG-(GPO)3-CONH₂ |
| SEQ ID NO: 411 | Cyanine 5-phycoerythrin-G3-(GfO)3-GPQGIAGQG-(GPO)3-CONH₂ |
| SEQ ID NO: 412 | Cyanine 5.5-phycoerythrin-G3-(GfO)3-GPQGIAGQG-(GPO)3-CONH₂ |
| SEQ ID NO: 413 | Cyanine 7-phycoerythrin-G3-(GfO)3-GPQGIAGQG-(GPO)3-CONH₂ |
| SEQ ID NO: 414 | Cyanine 7.5-phycoerythrin-G3-(GfO)3-GPQGIAGQG-(GPO)3-CONH₂ |
| SEQ ID NO: 415 | Rhodamine-G3-(GfO)3-GPQGIAGQG-(GPO)3-CONH₂ |
| SEQ ID NO: 416 | Sulfo-Cyanine3-G3-(GfO)3-GPQGIAGQG-(GPO)3-CONH₂ |
| SEQ ID NO: 417 | Sulfo-Cyanine5-G3-(GfO)3-GPQGIAGQG-(GPO)3-CONH₂ |
| SEQ ID NO: 418 | Sulfo-Cyanine5.5-G3-(GfO)3-GPQGIAGQG-(GPO)3-CONH₂ |
| SEQ ID NO: 419 | Sulfo-Cyanine7-G3-(GfO)3-GPQGIAGQG-(GPO)3-CONH₂ |
| SEQ ID NO: 420 | Sulfo-Cyanine7.5-G3-(GfO)3-GPQGIAGQG-(GPO)3-CONH₂ |
| SEQ ID NO: 421 | Texas Red-G3-(GfO)3-GPQGIAGQG-(GPO)3-CONH₂ |
| SEQ ID NO: 422 | Biotin-G3-(GfO)3-GPQGIAGQG-(GPO)₂-CONH₂ |
| SEQ ID NO: 423 | 5(6)-Carboxyfluorescein-G3-(GfO)3-GPQGIAGQG-(GPO)₂-CONH₂ |
| SEQ ID NO: 424 | Alexa Fluor-350-G3-(GfO)3-GPQGIAGQG-(GPO)2-CONH₂ |
| SEQ ID NO: 425 | Alexa Fluor-430-G3-(GfO)3-GPQGIAGQG-(GPO)2-CONH₂ |
| SEQ ID NO: 426 | Alexa Fluor-488-G3-(GfO)3-GPQGIAGQG-(GPO)2-CONH₂ |
| SEQ ID NO: 427 | Alexa Fluor-546-G3-(GfO)3-GPQGIAGQG-(GPO)2-CONH₂ |
| SEQ ID NO: 428 | Alexa Fluor-568-G3-(GfO)3-GPQGIAGQG-(GPO)2-CONH₂ |
| SEQ ID NO: 429 | Alexa Fluor-594-G3-(GfO)3-GPQGIAGQG-(GPO)2-CONH₂ |
| SEQ ID NO: 430 | Alexa Fluor-633-G3-(GfO)3-GPQGIAGQG-(GPO)2-CONH₂ |
| SEQ ID NO: 431 | Alexa Fluor-660-G3-(GfO)3-GPQGIAGQG-(GPO)2-CONH₂ |
| SEQ ID NO: 432 | Alexa Fluor-680-G3-(GfO)3-GPQGIAGQG-(GPO)2-CONH₂ |
| SEQ ID NO: 433 | Cyanine 3-G3-(GfO)3-GPQGIAGQG-(GPO)2-CONH₂ |
| SEQ ID NO: 434 | Cyanine 5-G3-(GfO)3-GPQGIAGQG-(GPO)2-CONH₂ |
| SEQ ID NO: 435 | Cyanine 5.5-G3-(GfO)3-GPQGIAGQG-(GPO)2-CONH₂ |
| SEQ ID NO: 436 | Cyanine 7-G3-(GfO)3-GPQGIAGQG-(GPO)2-CONH₂ |
| SEQ ID NO: 437 | Cyanine 7.5-G3-(GfO)3-GPQGIAGQG-(GPO)2-CONH₂ |
| SEQ ID NO: 438 | Cyanine 3-phycoerythrin-G3-(GfO)3-GPQGIAGQG-(GPO)2-CONH₂ |
| SEQ ID NO: 439 | Cyanine 5-phycoerythrin-G3-(GfO)3-GPQGIAGQG-(GPO)2-CONH₂ |
| SEQ ID NO: 440 | Cyanine 5.5-phycoerythrin-G3-(GfO)3-GPQGIAGQG-(GPO)2-CONH₂ |
| SEQ ID NO: 441 | Cyanine 7-phycoerythrin-G3-(GfO)3-GPQGIAGQG-(GPO)2-CONH₂ |
| SEQ ID NO: 442 | Cyanine 7.5-phycoerythrin-G3-(GfO)3-GPQGIAGQG-(GPO)2-CONH₂ |
| SEQ ID NO: 443 | Rhodamine-G3-(GfO)3-GPQGIAGQG-(GPO)2-CONH₂ |
| SEQ ID NO: 444 | Sulfo-Cyanine3-G3-(GfO)3-GPQGIAGQG-(GPO)2-CONH₂ |
| SEQ ID NO: 445 | Sulfo-Cyanine5-G3-(GfO)3-GPQGIAGQG-(GPO)2-CONH₂ |
| SEQ ID NO: 446 | Sulfo-Cyanine5.5-G3-(GfO)3-GPQGIAGQG-(GPO)2-CONH₂ |
| SEQ ID NO: 447 | Sulfo-Cyanine7-G3-(GfO)3-GPQGIAGQG-(GPO)2-CONH₂ |
| SEQ ID NO: 448 | Sulfo-Cyanine7.5-G3-(GfO)3-GPQGIAGQG-(GPO)2-CONH₂ |
| SEQ ID NO: 449 | Texas Red-G3-(GfO)3-GPQGIAGQG-(GPO)2-CONH₂ |
| SEQ ID NO: 450 | Biotin-G3-(GfO)3-GPQGIAGQG-(GfO)2-CONH₂ |
| SEQ ID NO: 451 | 5(6)-Carboxyfluorescein-G3-(GfO)3-GPQGIAGQG-(GfO)2-CONH₂ |
| SEQ ID NO: 452 | Alexa Fluor-350-G3-(GfO)3-GPQGIAGQG-(GfO)2-CONH₂ |
| SEQ ID NO: 453 | Alexa Fluor-430-G3-(GfO)3-GPQGIAGQG-(GfO)2-CONH₂ |
| SEQ ID NO: 454 | Alexa Fluor-488-G3-(GfO)3-GPQGIAGQG-(GfO)2-CONH₂ |
| SEQ ID NO: 455 | Alexa Fluor-546-G3-(GfO)3-GPQGIAGQG-(GfO)2-CONH₂ |
| SEQ ID NO: 456 | Alexa Fluor-568-G3-(GfO)3-GPQGIAGQG-(GfO)2-CONH₂ |
| SEQ ID NO: 457 | Alexa Fluor-594-G3-(GfO)3-GPQGIAGQG-(GfO)2-CONH₂ |
| SEQ ID NO: 458 | Alexa Fluor-633-G3-(GfO)3-GPQGIAGQG-(GfO)2-CONH₂ |
| SEQ ID NO: 459 | Alexa Fluor-660-G3-(GfO)3-GPQGIAGQG-(GfO)2-CONH₂ |
| SEQ ID NO: 460 | Alexa Fluor-680-G3-(GfO)3-GPQGIAGQG-(GfO)2-CONH₂ |
| SEQ ID NO: 461 | Cyanine 3-G3-(GfO)3-GPQGIAGQG-(GfO)2-CONH₂ |
| SEQ ID NO: 462 | Cyanine 5-G3-(GfO)3-GPQGIAGQG-(GfO)2-CONH₂ |
| SEQ ID NO: 463 | Cyanine 5.5-G3-(GfO)3-GPQGIAGQG-(GfO)2-CONH₂ |
| SEQ ID NO: 464 | Cyanine 7-G3-(GfO)3-GPQGIAGQG-(GfO)2-CONH₂ |
| SEQ ID NO: 465 | Cyanine 7.5-G3-(GfO)3-GPQGIAGQG-(GfO)2-CONH₂ |
| SEQ ID NO: 466 | Cyanine 3-phycoerythrin-G3-(GfO)3-GPQGIAGQG-(GfO)2-CONH₂ |
| SEQ ID NO: 467 | Cyanine 5-phycoerythrin-G3-(GfO)3-GPQGIAGQG-(GfO)2-CONH₂ |
| SEQ ID NO: 468 | Cyanine 5.5-phycoerythrin-G3-(GfO)3-GPQGIAGQG-(GfO)2-CONH₂ |
| SEQ ID NO: 469 | Cyanine 7-phycoerythrin-G3-(GfO)3-GPQGIAGQG-(GfO)2-CONH₂ |
| SEQ ID NO: 470 | Cyanine 7.5-phycoerythrin-G3-(GfO)3-GPQGIAGQG-(GfO)2-CONH₂ |
| SEQ ID NO: 471 | Rhodamine-G3-(GfO)3-GPQGIAGQG-(GfO)2-CONH₂ |
| SEQ ID NO: 472 | Sulfo-Cyanine3-G3-(GfO)3-GPQGIAGQG-(GfO)2-CONH₂ |
| SEQ ID NO: 473 | Sulfo-Cyanine5-G3-(GfO)3-GPQGIAGQG-(GfO)2-CONH₂ |
| SEQ ID NO: 474 | Sulfo-Cyanine5.5-G3-(GfO)3-GPQGIAGQG-(GfO)2-CONH₂ |
| SEQ ID NO: 475 | Sulfo-Cyanine7-G3-(GfO)3-GPQGIAGQG-(GfO)2-CONH₂ |
| SEQ ID NO: 476 | Sulfo-Cyanine7.5-G3-(GfO)3-GPQGIAGQG-(GfO)2-CONH₂ |
| SEQ ID NO: 477 | Texas Red-G3-(GfO)3-GPQGIAGQG-(GfO)2-CONH₂ |
| SEQ ID NO: 478 | Biotin-G3-(GfO)3-GPQGIAGQG-(GfO)3-CONH₂ |
| SEQ ID NO: 479 | 5(6)-Carboxyfluorescein-G3-(GfO)3-GPQGIAGQG-(GfO)3-CONH₂ |
| SEQ ID NO: 480 | Alexa Fluor-350-G3-(GfO)3-GPQGIAGQG-(GfO)3-CONH₂ |
| SEQ ID NO: 481 | Alexa Fluor-430-G3-(GfO)3-GPQGIAGQG-(GfO)3-CONH₂ |
| SEQ ID NO: 482 | Alexa Fluor-488-G3-(GfO)3-GPQGIAGQG-(GfO)3-CONH₂ |
| SEQ ID NO: 483 | Alexa Fluor-546-G3-(GfO)3-GPQGIAGQG-(GfO)3-CONH₂ |
| SEQ ID NO: 484 | Alexa Fluor-568-G3-(GfO)3-GPQGIAGQG-(GfO)3-CONH₂ |
| SEQ ID NO: 485 | Alexa Fluor-594-G3-(GfO)3-GPQGIAGQG-(GfO)3-CONH₂ |
| SEQ ID NO: 486 | Alexa Fluor-633-G3-(GfO)3-GPQGIAGQG-(GfO)3-CONH₂ |
| SEQ ID NO: 487 | Alexa Fluor-660-G3-(GfO)3-GPQGIAGQG-(GfO)3-CONH₂ |
| SEQ ID NO: 488 | Alexa Fluor-680-G3-(GfO)3-GPQGIAGQG-(GfO)3-CONH₂ |
| SEQ ID NO: 489 | Cyanine 3-G3-(GfO)3-GPQGIAGQG-(GfO)3-CONH₂ |
| SEQ ID NO: 490 | Cyanine 5-G3-(GfO)3-GPQGIAGQG-(GfO)3-CONH₂ |
| SEQ ID NO: 491 | Cyanine 5.5-G3-(GfO)3-GPQGIAGQG-(GfO)3-CONH₂ |
| SEQ ID NO: 492 | Cyanine 7-G3-(GfO)3-GPQGIAGQG-(GfO)3-CONH₂ |
| SEQ ID NO: 493 | Cyanine 7.5-G3-(GfO)3-GPQGIAGQG-(GfO)3-CONH₂ |
| SEQ ID NO: 494 | Cyanine 3-phycoerythrin-G3-(GfO)3-GPQGIAGQG-(GfO)3-CONH₂ |
| SEQ ID NO: 495 | Cyanine 5-phycoerythrin-G3-(GfO)3-GPQGIAGQG-(GfO)3-CONH₂ |
| SEQ ID NO: 496 | Cyanine 5.5-phycoerythrin-G3-(GfO)3-GPQGIAGQG-(GfO)3-CONH₂ |
| SEQ ID NO: 497 | Cyanine 7-phycoerythrin-G3-(GfO)3-GPQGIAGQG-(GfO)3-CONH₂ |
| SEQ ID NO: 498 | Cyanine 7.5-phycoerythrin-G3-(GfO)3-GPQGIAGQG-(GfO)3-CONH₂ |
| SEQ ID NO: 499 | Rhodamine-G3-(GfO)3-GPQGIAGQG-(GfO)3-CONH₂ |
| SEQ ID NO: 500 | Sulfo-Cyanine3-G3-(GfO)3-GPQGIAGQG-(GfO)3-CONH₂ |
| SEQ ID NO: 501 | Sulfo-Cyanine5-G3-(GfO)3-GPQGIAGQG-(GfO)3-CONH₂ |
| SEQ ID NO: 502 | Sulfo-Cyanine5.5-G3-(GfO)3-GPQGIAGQG-(GfO)3-CONH₂ |
| SEQ ID NO: 503 | Sulfo-Cyanine7-G3-(GfO)3-GPQGIAGQG-(GfO)3-CONH₂ |
| SEQ ID NO: 504 | Sulfo-Cyanine7.5-G3-(GfO)3-GPQGIAGQG-(GfO)3-CONH₂ |
| SEQ ID NO: 505 | Texas Red-G3-(GfO)3-GPQGIAGQG-(GfO)3-CONH₂ |
| SEQ ID NO: 506 | Biotin-G3-(GPO)3-GPLGIAGIG-(GPO)2-CONH₂ |
| SEQ ID NO: 507 | 5(6)-Carboxyfluorescein-G3-(GPO)3-GPLGIAGIG-(GPO)2-CONH₂ |
| SEQ ID NO: 508 | Alexa Fluor-350-G3-(GPO)3-GPLGIAGIG-(GPO)2-CONH₂ |
| SEQ ID NO: 509 | Alexa Fluor-430-G3-(GPO)3-GPLGIAGIG-(GPO)2-CONH₂ |
| SEQ ID NO: 510 | Alexa Fluor-488-G3-(GPO)3-GPLGIAGIG-(GPO)2-CONH₂ |
| SEQ ID NO: 511 | Alexa Fluor-546-G3-(GPO)3-GPLGIAGIG-(GPO)2-CONH₂ |
| SEQ ID NO: 512 | Alexa Fluor-568-G3-(GPO)3-GPLGIAGIG-(GPO)2-CONH₂ |
| SEQ ID NO: 513 | Alexa Fluor-594-G3-(GPO)3-GPLGIAGIG-(GPO)2-CONH₂ |
| SEQ ID NO: 514 | Alexa Fluor-633-G3-(GPO)3-GPLGIAGIG-(GPO)2-CONH₂ |
| SEQ ID NO: 515 | Alexa Fluor-660-G3-(GPO)3-GPLGIAGIG-(GPO)2-CONH₂ |
| SEQ ID NO: 516 | Alexa Fluor-680-G3-(GPO)3-GPLGIAGIG-(GPO)2-CONH₂ |
| SEQ ID NO: 517 | Cyanine 3-G3-(GPO)3-GPLGIAGIG-(GPO)2-CONH₂ |
| SEQ ID NO: 518 | Cyanine 5-G3-(GPO)3-GPLGIAGIG-(GPO)2-CONH₂ |
| SEQ ID NO: 519 | Cyanine 5.5-G3-(GPO)3-GPLGIAGIG-(GPO)2-CONH₂ |
| SEQ ID NO: 520 | Cyanine 7-G3-(GPO)3-GPLGIAGIG-(GPO)2-CONH₂ |
| SEQ ID NO: 521 | Cyanine 7.5-G3-(GPO)3-GPLGIAGIG-(GPO)2-CONH₂ |
| SEQ ID NO: 522 | Cyanine 3-phycoerythrin-G3-(GPO)3-GPLGIAGIG-(GPO)2-CONH₂ |
| SEQ ID NO: 523 | Cyanine 5-phycoerythrin-G3-(GPO)3-GPLGIAGIG-(GPO)2-CONH₂ |
| SEQ ID NO: 524 | Cyanine 5.5-phycoerythrin-G3-(GPO)3-GPLGIAGIG-(GPO)2-CONH₂ |
| SEQ ID NO: 525 | Cyanine 7-phycoerythrin-G3-(GPO)3-GPLGIAGIG-(GPO)2-CONH₂ |
| SEQ ID NO: 526 | Cyanine 7.5-phycoerythrin-G3-(GPO)3-GPLGIAGIG-(GPO)2-CONH₂ |
| SEQ ID NO: 527 | Rhodamine-G3-(GPO)3-GPLGIAGIG-(GPO)2-CONH₂ |
| SEQ ID NO: 528 | Sulfo-Cyanine3-G3-(GPO)3-GPLGIAGIG-(GPO)2-CONH₂ |
| SEQ ID NO: 529 | Sulfo-Cyanine5-G3-(GPO)3-GPLGIAGIG-(GPO)2-CONH₂ |
| SEQ ID NO: 530 | Sulfo-Cyanine5.5-G3-(GPO)3-GPLGIAGIG-(GPO)2-CONH₂ |
| SEQ ID NO: 531 | Sulfo-Cyanine7-G3-(GPO)3-GPLGIAGIG-(GPO)2-CONH₂ |
| SEQ ID NO: 532 | Sulfo-Cyanine7.5-G3-(GPO)3-GPLGIAGIG-(GPO)2-CONH₂ |
| SEQ ID NO: 533 | Texas Red-G3-(GPO)3-GPLGIAGIG-(GPO)2-CONH₂ |
| SEQ ID NO: 534 | Biotin-G3-(GPO)3-GPLGIAGIG-(GPO)3-CONH₂ |
| SEQ ID NO: 535 | 5(6)-Carboxyfluorescein-G3-(GPO)3-GPLGIAGIG-(GPO)3-CONH₂ |
| SEQ ID NO: 536 | Alexa Fluor-350-G3-(GPO)3-GPLGIAGIG-(GPO)3-CONH₂ |
| SEQ ID NO: 537 | Alexa Fluor-430-G3-(GPO)3-GPLGIAGIG-(GPO)3-CONH₂ |
| SEQ ID NO: 538 | Alexa Fluor-488-G3-(GPO)3-GPLGIAGIG-(GPO)3-CONH₂ |
| SEQ ID NO: 539 | Alexa Fluor-546-G3-(GPO)3-GPLGIAGIG-(GPO)3-CONH₂ |
| SEQ ID NO: 540 | Alexa Fluor-568-G3-(GPO)3-GPLGIAGIG-(GPO)3-CONH₂ |
| SEQ ID NO: 541 | Alexa Fluor-594-G3-(GPO)3-GPLGIAGIG-(GPO)3-CONH₂ |
| SEQ ID NO: 542 | Alexa Fluor-633-G3-(GPO)3-GPLGIAGIG-(GPO)3-CONH₂ |
| SEQ ID NO: 543 | Alexa Fluor-660-G3-(GPO)3-GPLGIAGIG-(GPO)3-CONH₂ |
| SEQ ID NO: 544 | Alexa Fluor-680-G3-(GPO)3-GPLGIAGIG-(GPO)3-CONH₂ |
| SEQ ID NO: 545 | Cyanine 3-G3-(GPO)3-GPLGIAGIG-(GPO)3-CONH₂ |
| SEQ ID NO: 546 | Cyanine 5-G3-(GPO)3-GPLGIAGIG-(GPO)3-CONH₂ |
| SEQ ID NO: 547 | Cyanine 5.5-G3-(GPO)3-GPLGIAGIG-(GPO)3-CONH₂ |
| SEQ ID NO: 548 | Cyanine 7-G3-(GPO)3-GPLGIAGIG-(GPO)3-CONH₂ |
| SEQ ID NO: 549 | Cyanine 7.5-G3-(GPO)3-GPLGIAGIG-(GPO)3-CONH₂ |
| SEQ ID NO: 550 | Cyanine 3-phycoerythrin-G3-(GPO)3-GPLGIAGIG-(GPO)3-CONH₂ |
| SEQ ID NO: 551 | Cyanine 5-phycoerythrin-G3-(GPO)3-GPLGIAGIG-(GPO)3-CONH₂ |
| SEQ ID NO: 552 | Cyanine 5.5-phycoerythrin-G3-(GPO)3-GPLGIAGIG-(GPO)3-CONH₂ |
| SEQ ID NO: 553 | Cyanine 7-phycoerythrin-G3-(GPO)3-GPLGIAGIG-(GPO)3-CONH₂ |
| SEQ ID NO: 554 | Cyanine 7.5-phycoerythrin-G3-(GPO)3-GPLGIAGIG-(GPO)3-CONH₂ |
| SEQ ID NO: 555 | Rhodamine-G3-(GPO)3-GPLGIAGIG-(GPO)3-CONH₂ |
| SEQ ID NO: 556 | Sulfo-Cyanine3-G3-(GPO)3-GPLGIAGIG-(GPO)3-CONH₂ |
| SEQ ID NO: 557 | Sulfo-Cyanine5-G3-(GPO)3-GPLGIAGIG-(GPO)3-CONH₂ |
| SEQ ID NO: 558 | Sulfo-Cyanine5.5-G3-(GPO)3-GPLGIAGIG-(GPO)3-CONH₂ |
| SEQ ID NO: 559 | Sulfo-Cyanine7-G3-(GPO)3-GPLGIAGIG-(GPO)3-CONH₂ |
| SEQ ID NO: 560 | Sulfo-Cyanine7.5-G3-(GPO)3-GPLGIAGIG-(GPO)3-CONH₂ |
| SEQ ID NO: 561 | Texas Red-G3-(GPO)3-GPLGIAGIG-(GPO)3-CONH₂ |
| SEQ ID NO: 562 | Biotin-G3-(GfO)3-GPLGIAGIG-(GPO)3-CONH₂ |
| SEQ ID NO: 563 | 5(6)-Carboxyfluorescein-G3-(GfO)3-GPLGIAGIG-(GPO)3-CONH₂ |
| SEQ ID NO: 564 | Alexa Fluor-350-G3-(GfO)3-GPLGIAGIG-(GPO)3-CONH₂ |
| SEQ ID NO: 565 | Alexa Fluor-430-G3-(GfO)3-GPLGIAGIG-(GPO)3-CONH₂ |
| SEQ ID NO: 566 | Alexa Fluor-488-G3-(GfO)3-GPLGIAGIG-(GPO)3-CONH₂ |
| SEQ ID NO: 567 | Alexa Fluor-546-G3-(GfO)3-GPLGIAGIG-(GPO)3-CONH₂ |
| SEQ ID NO: 568 | Alexa Fluor-568-G3-(GfO)3-GPLGIAGIG-(GPO)3-CONH₂ |
| SEQ ID NO: 569 | Alexa Fluor-594-G3-(GfO)3-GPLGIAGIG-(GPO)3-CONH₂ |
| SEQ ID NO: 570 | Alexa Fluor-633-G3-(GfO)3-GPLGIAGIG-(GPO)3-CONH₂ |
| SEQ ID NO: 571 | Alexa Fluor-660-G3-(GfO)3-GPLGIAGIG-(GPO)3-CONH₂ |
| SEQ ID NO: 572 | Alexa Fluor-680-G3-(GfO)3-GPLGIAGIG-(GPO)3-CONH₂ |
| SEQ ID NO: 573 | Cyanine 3-G3-(GfO)3-GPLGIAGIG-(GPO)3-CONH₂ |
| SEQ ID NO: 574 | Cyanine 5-G3-(GfO)3-GPLGIAGIG-(GPO)3-CONH₂ |
| SEQ ID NO: 575 | Cyanine 5.5-G3-(GfO)3-GPLGIAGIG-(GPO)3-CONH₂ |
| SEQ ID NO: 576 | Cyanine 7-G3-(GfO)3-GPLGIAGIG-(GPO)3-CONH₂ |
| SEQ ID NO: 577 | Cyanine 7.5-G3-(GfO)3-GPLGIAGIG-(GPO)3-CONH₂ |
| SEQ ID NO: 578 | Cyanine 3-phycoerythrin-G3-(GfO)3-GPLGIAGIG-(GPO)3-CONH₂ |
| SEQ ID NO: 579 | Cyanine 5-phycoerythrin-G3-(GfO)3-GPLGIAGIG-(GPO)3-CONH₂ |
| SEQ ID NO: 580 | Cyanine 5.5-phycoerythrin-G3-(GfO)3-GPLGIAGIG-(GPO)3-CONH₂ |
| SEQ ID NO: 581 | Cyanine 7-phycoerythrin-G3-(GfO)3-GPLGIAGIG-(GPO)3-CONH₂ |
| SEQ ID NO: 582 | Cyanine 7.5-phycoerythrin-G3-(GfO)3-GPLGIAGIG-(GPO)3-CONH₂ |
| SEQ ID NO: 583 | Rhodamine-G3-(GfO)3-GPLGIAGIG-(GPO)3-CONH₂ |
| SEQ ID NO: 584 | Sulfo-Cyanine3-G3-(GfO)3-GPLGIAGIG-(GPO)3-CONH₂ |
| SEQ ID NO: 585 | Sulfo-Cyanine5-G3-(GfO)3-GPLGIAGIG-(GPO)3-CONH₂ |
| SEQ ID NO: 586 | Sulfo-Cyanine5.5-G3-(GfO)3-GPLGIAGIG-(GPO)3-CONH₂ |
| SEQ ID NO: 587 | Sulfo-Cyanine7-G3-(GfO)3-GPLGIAGIG-(GPO)3-CONH₂ |
| SEQ ID NO: 588 | Sulfo-Cyanine7.5-G3-(GfO)3-GPLGIAGIG-(GPO)3-CONH₂ |
| SEQ ID NO: 589 | Texas Red-G3-(GfO)3-GPLGIAGIG-(GPO)3-CONH₂ |
| SEQ ID NO: 590 | Biotin-G3-(GfO)3-GPLGIAGIG-(GPO)2-CONH₂ |
| SEQ ID NO: 591 | 5(6)-Carboxyfluorescein-G3-(GfO)3-GPLGIAGIG-(GPO)₂-CONH₂ |
| SEQ ID NO: 592 | Alexa Fluor-350-G3-(GfO)3-GPLGIAGIG-(GPO)2-CONH₂ |
| SEQ ID NO: 593 | Alexa Fluor-430-G3-(GfO)3-GPLGIAGIG-(GPO)2-CONH₂ |
| SEQ ID NO: 594 | Alexa Fluor-488-G3-(GfO)3-GPLGIAGIG-(GPO)2-CONH₂ |
| SEQ ID NO: 595 | Alexa Fluor-546-G3-(GfO)3-GPLGIAGIG-(GPO)2-CONH₂ |
| SEQ ID NO: 596 | Alexa Fluor-568-G3-(GfO)3-GPLGIAGIG-(GPO)2-CONH₂ |
| SEQ ID NO: 597 | Alexa Fluor-594-G3-(GfO)3-GPLGIAGIG-(GPO)2-CONH₂ |
| SEQ ID NO: 598 | Alexa Fluor-633-G3-(GfO)3-GPLGIAGIG-(GPO)2-CONH₂ |
| SEQ ID NO: 599 | Alexa Fluor-660-G3-(GfO)3-GPLGIAGIG-(GPO)2-CONH₂ |
| SEQ ID NO: 600 | Alexa Fluor-680-G3-(GfO)3-GPLGIAGIG-(GPO)2-CONH₂ |
| SEQ ID NO: 601 | Cyanine 3-G3-(GfO)3-GPLGIAGIG-(GPO)2-CONH₂ |
| SEQ ID NO: 602 | Cyanine 5-G3-(GfO)3-GPLGIAGIG-(GPO)2-CONH₂ |
| SEQ ID NO: 603 | Cyanine 5.5-G3-(GfO)3-GPLGIAGIG-(GPO)2-CONH₂ |
| SEQ ID NO: 604 | Cyanine 7-G3-(GfO)3-GPLGIAGIG-(GPO)2-CONH₂ |
| SEQ ID NO: 605 | Cyanine 7.5-G3-(GfO)3-GPLGIAGIG-(GPO)2-CONH₂ |
| SEQ ID NO: 606 | Cyanine 3-phycoerythrin-G3-(GfO)3-GPLGIAGIG-(GPO)2-CONH₂ |
| SEQ ID NO: 607 | Cyanine 5-phycoerythrin-G3-(GfO)3-GPLGIAGIG-(GPO)2-CONH₂ |
| SEQ ID NO: 608 | Cyanine 5.5-phycoerythrin-G3-(GfO)3-GPLGIAGIG-(GPO)2-CONH₂ |
| SEQ ID NO: 609 | Cyanine 7-phycoerythrin-G3-(GfO)3-GPLGIAGIG-(GPO)2-CONH₂ |
| SEQ ID NO: 610 | Cyanine 7.5-phycoerythrin-G3-(GfO)3-GPLGIAGIG-(GPO)2-CONH₂ |
| SEQ ID NO: 611 | Rhodamine-G3-(GfO)3-GPLGIAGIG-(GPO)2-CONH₂ |
| SEQ ID NO: 612 | Sulfo-Cyanine3-G3-(GfO)3-GPLGIAGIG-(GPO)2-CONH₂ |
| SEQ ID NO: 613 | Sulfo-Cyanine5-G3-(GfO)3-GPLGIAGIG-(GPO)2-CONH₂ |
| SEQ ID NO: 614 | Sulfo-Cyanine5.5-G3-(GfO)3-GPLGIAGIG-(GPO)2-CONH₂ |
| SEQ ID NO: 615 | Sulfo-Cyanine7-G3-(GfO)3-GPLGIAGIG-(GPO)2-CONH₂ |
| SEQ ID NO: 616 | Sulfo-Cyanine7.5-G3-(GfO)3-GPLGIAGIG-(GPO)2-CONH₂ |
| SEQ ID NO: 617 | Texas Red-G3-(GfO)3-GPLGIAGIG-(GPO)2-CONH₂ |
| SEQ ID NO: 618 | Biotin-G3-(GfO)3-GPLGIAGIG-(GfO)2-CONH₂ |
| SEQ ID NO: 619 | 5(6)-Carboxyfluorescein-G3-(GfO)3-GPLGIAGIG-(GfO)2-CONH₂ |
| SEQ ID NO: 620 | Alexa Fluor-350-G3-(GfO)3-GPLGIAGIG-(GfO)2-CONH₂ |
| SEQ ID NO: 621 | Alexa Fluor-430-G3-(GfO)3-GPLGIAGIG-(GfO)2-CONH₂ |
| SEQ ID NO: 622 | Alexa Fluor-488-G3-(GfO)3-GPLGIAGIG-(GfO)2-CONH₂ |
| SEQ ID NO: 623 | Alexa Fluor-546-G3-(GfO)3-GPLGIAGIG-(GfO)2-CONH₂ |
| SEQ ID NO: 624 | Alexa Fluor-568-G3-(GfO)3-GPLGIAGIG-(GfO)2-CONH₂ |
| SEQ ID NO: 625 | Alexa Fluor-594-G3-(GfO)3-GPLGIAGIG-(GfO)2-CONH₂ |
| SEQ ID NO: 626 | Alexa Fluor-633-G3-(GfO)3-GPLGIAGIG-(GfO)2-CONH₂ |
| SEQ ID NO: 627 | Alexa Fluor-660-G3-(GfO)3-GPLGIAGIG-(GfO)2-CONH₂ |
| SEQ ID NO: 628 | Alexa Fluor-680-G3-(GfO)3-GPLGIAGIG-(GfO)2-CONH₂ |
| SEQ ID NO: 629 | Cyanine 3-G3-(GfO)3-GPLGIAGIG-(GfO)2-CONH₂ |
| SEQ ID NO: 630 | Cyanine 5-G3-(GfO)3-GPLGIAGIG-(GfO)2-CONH₂ |
| SEQ ID NO: 631 | Cyanine 5.5-G3-(GfO)3-GPLGIAGIG-(GfO)2-CONH₂ |
| SEQ ID NO: 632 | Cyanine 7-G3-(GfO)3-GPLGIAGIG-(GfO)2-CONH₂ |
| SEQ ID NO: 633 | Cyanine 7.5-G3-(GfO)3-GPLGIAGIG-(GfO)2-CONH₂ |
| SEQ ID NO: 634 | Cyanine 3-phycoerythrin-G3-(GfO)3-GPLGIAGIG-(GfO)2-CONH₂ |
| SEQ ID NO: 635 | Cyanine 5-phycoerythrin-G3-(GfO)3-GPLGIAGIG-(GfO)2-CONH₂ |
| SEQ ID NO: 636 | Cyanine 5.5-phycoerythrin-G3-(GfO)3-GPLGIAGIG-(GfO)2-CONH₂ |
| SEQ ID NO: 637 | Cyanine 7-phycoerythrin-G3-(GfO)3-GPLGIAGIG-(GfO)2-CONH₂ |
| SEQ ID NO: 638 | Cyanine 7.5-phycoerythrin-G3-(GfO)3-GPLGIAGIG-(GfO)2-CONH₂ |
| SEQ ID NO: 639 | Rhodamine-G3-(GfO)3-GPLGIAGIG-(GfO)2-CONH₂ |
| SEQ ID NO: 640 | Sulfo-Cyanine3-G3-(GfO)3-GPLGIAGIG-(GfO)2-CONH₂ |
| SEQ ID NO: 641 | Sulfo-Cyanine5-G3-(GfO)3-GPLGIAGIG-(GfO)2-CONH₂ |
| SEQ ID NO: 642 | Sulfo-Cyanine5.5-G3-(GfO)3-GPLGIAGIG-(GfO)2-CONH₂ |
| SEQ ID NO: 643 | Sulfo-Cyanine7-G3-(GfO)3-GPLGIAGIG-(GfO)2-CONH₂ |
| SEQ ID NO: 644 | Sulfo-Cyanine7.5-G3-(GfO)3-GPLGIAGIG-(GfO)2-CONH₂ |
| SEQ ID NO: 645 | Texas Red-G3-(GfO)3-GPLGIAGIG-(GfO)2-CONH₂ |
| SEQ ID NO: 646 | Biotin-G3-(GfO)3-GPLGIAGIG-(GfO)3-CONH₂ |
| SEQ ID NO: 647 | 5(6)-Carboxyfluorescein-G3-(GfO)3-GPLGIAGIG-(GfO)3-CONH₂ |
| SEQ ID NO: 648 | Alexa Fluor-350-G3-(GfO)3-GPLGIAGIG-(GfO)3-CONH₂ |
| SEQ ID NO: 649 | Alexa Fluor-430-G3-(GfO)3-GPLGIAGIG-(GfO)3-CONH₂ |
| SEQ ID NO: 650 | Alexa Fluor-488-G3-(GfO)3-GPLGIAGIG-(GfO)3-CONH₂ |
| SEQ ID NO: 651 | Alexa Fluor-546-G3-(GfO)3-GPLGIAGIG-(GfO)3-CONH₂ |
| SEQ ID NO: 652 | Alexa Fluor-568-G3-(GfO)3-GPLGIAGIG-(GfO)3-CONH₂ |
| SEQ ID NO: 653 | Alexa Fluor-594-G3-(GfO)3-GPLGIAGIG-(GfO)3-CONH₂ |
| SEQ ID NO: 654 | Alexa Fluor-633-G3-(GfO)3-GPLGIAGIG-(GfO)3-CONH₂ |
| SEQ ID NO: 655 | Alexa Fluor-660-G3-(GfO)3-GPLGIAGIG-(GfO)3-CONH₂ |
| SEQ ID NO: 656 | Alexa Fluor-680-G3-(GfO)3-GPLGIAGIG-(GfO)3-CONH₂ |
| SEQ ID NO: 657 | Cyanine 3-G3-(GfO)3-GPLGIAGIG-(GfO)3-CONH₂ |
| SEQ ID NO: 658 | Cyanine 5-G3-(GfO)3-GPLGIAGIG-(GfO)3-CONH₂ |
| SEQ ID NO: 659 | Cyanine 5.5-G3-(GfO)3-GPLGIAGIG-(GfO)3-CONH₂ |
| SEQ ID NO: 660 | Cyanine 7-G3-(GfO)3-GPLGIAGIG-(GfO)3-CONH₂ |
| SEQ ID NO: 661 | Cyanine 7.5-G3-(GfO)3-GPLGIAGIG-(GfO)3-CONH₂ |
| SEQ ID NO: 662 | Cyanine 3-phycoerythrin-G3-(GfO)3-GPLGIAGIG-(GfO)3-CONH₂ |
| SEQ ID NO: 663 | Cyanine 5-phycoerythrin-G3-(GfO)3-GPLGIAGIG-(GfO)3-CONH₂ |
| SEQ ID NO: 664 | Cyanine 5.5-phycoerythrin-G3-(GfO)3-GPLGIAGIG-(GfO)3-CONH₂ |
| SEQ ID NO: 665 | Cyanine 7-phycoerythrin-G3-(GfO)3-GPLGIAGIG-(GfO)3-CONH₂ |
| SEQ ID NO: 666 | Cyanine 7.5-phycoerythrin-G3-(GfO)3-GPLGIAGIG-(GfO)3-CONH₂ |
| SEQ ID NO: 667 | Rhodamine-G3-(GfO)3-GPLGIAGIG-(GfO)3-CONH₂ |
| SEQ ID NO: 668 | Sulfo-Cyanine3-G3-(GfO)3-GPLGIAGIG-(GfO)3-CONH₂ |
| SEQ ID NO: 669 | Sulfo-Cyanine5-G3-(GfO)3-GPLGIAGIG-(GfO)3-CONH₂ |
| SEQ ID NO: 670 | Sulfo-Cyanine5.5-G3-(GfO)3-GPLGIAGIG-(GfO)3-CONH₂ |
| SEQ ID NO: 671 | Sulfo-Cyanine7-G3-(GfO)3-GPLGIAGIG-(GfO)3-CONH₂ |
| SEQ ID NO: 672 | Sulfo-Cyanine7.5-G3-(GfO)3-GPLGIAGIG-(GfO)3-CONH₂ |
| SEQ ID NO: 673 | Texas Red-G3-(GfO)3-GPLGIAGIG-(GfO)3-CONH₂ |
| SEQ ID NO: 674 | Biotin-G3-(GPO)3-GPLGIAGITGARGLA-(GPO)2-CONH₂ |
| SEQ ID NO: 675 | 5(6)-Carboxyfluorescein-G3-(GPO)3-GPLGIAGITGARGLA-(GPO)2-CONH₂ |
| SEQ ID NO: 676 | Alexa Fluor-350-G3-(GPO)3-GPLGIAGITGARGLA-(GPO)2-CONH₂ |
| SEQ ID NO: 677 | Alexa Fluor-430-G3-(GPO)3-GPLGIAGITGARGLA-(GPO)2-CONH₂ |
| SEQ ID NO: 678 | Alexa Fluor-488-G3-(GPO)3-GPLGIAGITGARGLA-(GPO)2-CONH₂ |
| SEQ ID NO: 679 | Alexa Fluor-546-G3-(GPO)3-GPLGIAGITGARGLA-(GPO)2-CONH₂ |
| SEQ ID NO: 680 | Alexa Fluor-568-G3-(GPO)3-GPLGIAGITGARGLA-(GPO)2-CONH₂ |
| SEQ ID NO: 681 | Alexa Fluor-594-G3-(GPO)3-GPLGIAGITGARGLA-(GPO)2-CONH₂ |
| SEQ ID NO: 682 | Alexa Fluor-633-G3-(GPO)3-GPLGIAGITGARGLA-(GPO)2-CONH₂ |
| SEQ ID NO: 683 | Alexa Fluor-660-G3-(GPO)3-GPLGIAGITGARGLA-(GPO)2-CONH₂ |
| SEQ ID NO: 684 | Alexa Fluor-680-G3-(GPO)3-GPLGIAGITGARGLA-(GPO)2-CONH₂ |
| SEQ ID NO: 685 | Cyanine 3-G3-(GPO)3-GPLGIAGITGARGLA-(GPO)2-CONH₂ |
| SEQ ID NO: 686 | Cyanine 5-G3-(GPO)3-GPLGIAGITGARGLA-(GPO)2-CONH₂ |
| SEQ ID NO: 687 | Cyanine 5.5-G3-(GPO)3-GPLGIAGITGARGLA-(GPO)2-CONH₂ |
| SEQ ID NO: 688 | Cyanine 7-G3-(GPO)3-GPLGIAGITGARGLA-(GPO)2-CONH₂ |
| SEQ ID NO: 689 | Cyanine 7.5-G3-(GPO)3-GPLGIAGITGARGLA-(GPO)2-CONH₂ |
| SEQ ID NO: 690 | Cyanine 3-phycoerythrin-G3-(GPO)3-GPLGIAGITGARGLA-(GPO)2-CONH₂ |
| SEQ ID NO: 691 | Cyanine 5-phycoerythrin-G3-(GPO)3-GPLGIAGITGARGLA-(GPO)2-CONH₂ |
| SEQ ID NO: 692 | Cyanine 5.5-phycoerythrin-G3-(GPO)3-GPLGIAGITGARGLA-(GPO)2-CONH₂ |
| SEQ ID NO: 693 | Cyanine 7-phycoerythrin-G3-(GPO)3-GPLGIAGITGARGLA-(GPO)2-CONH₂ |
| SEQ ID NO: 694 | Cyanine 7.5-phycoerythrin-G3-(GPO)3-GPLGIAGITGARGLA-(GPO)2-CONH₂ |
| SEQ ID NO: 695 | Rhodamine-G3-(GPO)3-GPLGIAGITGARGLA-(GPO)2-CONH₂ |
| SEQ ID NO: 696 | Sulfo-Cyanine3-G3-(GPO)3-GPLGIAGITGARGLA-(GPO)2-CONH₂ |
| SEQ ID NO: 697 | Sulfo-Cyanine5-G3-(GPO)3-GPLGIAGITGARGLA-(GPO)2-CONH₂ |
| SEQ ID NO: 698 | Sulfo-Cyanine5.5-G3-(GPO)3-GPLGIAGITGARGLA-(GPO)2-CONH₂ |
| SEQ ID NO: 699 | Sulfo-Cyanine7-G3-(GPO)3-GPLGIAGITGARGLA-(GPO)2-CONH₂ |
| SEQ ID NO: 700 | Sulfo-Cyanine7.5-G3-(GPO)3-GPLGIAGITGARGLA-(GPO)2-CONH₂ |
| SEQ ID NO: 701 | Texas Red-G3-(GPO)3-GPLGIAGITGARGLA-(GPO)2-CONH₂ |
| SEQ ID NO: 702 | Biotin-G3-(GPO)3-GPLGIAGITGARGLA-(GPO)3-CONH₂ |
| SEQ ID NO: 703 | 5(6)-Carboxyfluorescein-G3-(GPO)3-GPLGIAGITGARGLA-(GPO)3-CONH₂ |
| SEQ ID NO: 704 | Alexa Fluor-350-G3-(GPO)3-GPLGIAGITGARGLA-(GPO)3-CONH₂ |
| SEQ ID NO: 705 | Alexa Fluor-430-G3-(GPO)3-GPLGIAGITGARGLA-(GPO)3-CONH₂ |
| SEQ ID NO: 706 | Alexa Fluor-488-G3-(GPO)3-GPLGIAGITGARGLA-(GPO)3-CONH₂ |
| SEQ ID NO: 707 | Alexa Fluor-546-G3-(GPO)3-GPLGIAGITGARGLA-(GPO)3-CONH₂ |
| SEQ ID NO: 708 | Alexa Fluor-568-G3-(GPO)3-GPLGIAGITGARGLA-(GPO)3-CONH₂ |
| SEQ ID NO: 709 | Alexa Fluor-594-G3-(GPO)3-GPLGIAGITGARGLA-(GPO)3-CONH₂ |
| SEQ ID NO: 710 | Alexa Fluor-633-G3-(GPO)3-GPLGIAGITGARGLA-(GPO)3-CONH₂ |
| SEQ ID NO: 711 | Alexa Fluor-660-G3-(GPO)3-GPLGIAGITGARGLA-(GPO)3-CONH₂ |
| SEQ ID NO: 712 | Alexa Fluor-680-G3-(GPO)3-GPLGIAGITGARGLA-(GPO)3-CONH₂ |
| SEQ ID NO: 713 | Cyanine 3-G3-(GPO)3-GPLGIAGITGARGLA-(GPO)3-CONH₂ |
| SEQ ID NO: 714 | Cyanine 5-G3-(GPO)3-GPLGIAGITGARGLA-(GPO)3-CONH₂ |
| SEQ ID NO: 715 | Cyanine 5.5-G3-(GPO)3-GPLGIAGITGARGLA-(GPO)3-CONH₂ |
| SEQ ID NO: 716 | Cyanine 7-G3-(GPO)3-GPLGIAGITGARGLA-(GPO)3-CONH₂ |
| SEQ ID NO: 717 | Cyanine 7.5-G3-(GPO)3-GPLGIAGITGARGLA-(GPO)3-CONH₂ |
| SEQ ID NO: 718 | Cyanine 3-phycoerythrin-G3-(GPO)3-GPLGIAGITGARGLA-(GPO)3-CONH₂ |
| SEQ ID NO: 719 | Cyanine 5-phycoerythrin-G3-(GPO)3-GPLGIAGITGARGLA-(GPO)3-CONH₂ |
| SEQ ID NO: 720 | Cyanine 5.5-phycoerythrin-G3-(GPO)3-GPLGIAGITGARGLA-(GPO)3-CONH₂ |
| SEQ ID NO: 721 | Cyanine 7-phycoerythrin-G3-(GPO)3-GPLGIAGITGARGLA-(GPO)3-CONH₂ |
| SEQ ID NO: 722 | Cyanine 7.5-phycoerythrin-G3-(GPO)3-GPLGIAGITGARGLA-(GPO)3-CONH₂ |
| SEQ ID NO: 723 | Rhodamine-G3-(GPO)3-GPLGIAGITGARGLA-(GPO)3-CONH₂ |
| SEQ ID NO: 724 | Sulfo-Cyanine3-G3-(GPO)3-GPLGIAGITGARGLA-(GPO)3-CONH₂ |
| SEQ ID NO: 725 | Sulfo-Cyanine5-G3-(GPO)3-GPLGIAGITGARGLA-(GPO)3-CONH₂ |
| SEQ ID NO: 726 | Sulfo-Cyanine5.5-G3-(GPO)3-GPLGIAGITGARGLA-(GPO)3-CONH₂ |
| SEQ ID NO: 727 | Sulfo-Cyanine7-G3-(GPO)3-GPLGIAGITGARGLA-(GPO)3-CONH₂ |
| SEQ ID NO: 728 | Sulfo-Cyanine7.5-G3-(GPO)3-GPLGIAGITGARGLA-(GPO)3-CONH₂ |
| SEQ ID NO: 729 | Texas Red-G3-(GPO)3-GPLGIAGITGARGLA-(GPO)3-CONH₂ |
| SEQ ID NO: 730 | Biotin-G3-(GfO)3-GPLGIAGITGARGLA-(GPO)3-CONH₂ |
| SEQ ID NO: 731 | 5(6)-Carboxyfluorescein-G3-(GfO)3-GPLGIAGITGARGLA-(GPO)3-CONH₂ |
| SEQ ID NO: 732 | Alexa Fluor-350-G3-(GfO)3-GPLGIAGITGARGLA-(GPO)3-CONH₂ |
| SEQ ID NO: 733 | Alexa Fluor-430-G3-(GfO)3-GPLGIAGITGARGLA-(GPO)3-CONH₂ |
| SEQ ID NO: 734 | Alexa Fluor-488-G3-(GfO)3-GPLGIAGITGARGLA-(GPO)3-CONH₂ |
| SEQ ID NO: 735 | Alexa Fluor-546-G3-(GfO)3-GPLGIAGITGARGLA-(GPO)3-CONH₂ |
| SEQ ID NO: 736 | Alexa Fluor-568-G3-(GfO)3-GPLGIAGITGARGLA-(GPO)3-CONH₂ |
| SEQ ID NO: 737 | Alexa Fluor-594-G3-(GfO)3-GPLGIAGITGARGLA-(GPO)3-CONH₂ |
| SEQ ID NO: 738 | Alexa Fluor-633-G3-(GfO)3-GPLGIAGITGARGLA-(GPO)3-CONH₂ |
| SEQ ID NO: 739 | Alexa Fluor-660-G3-(GfO)3-GPLGIAGITGARGLA-(GPO)3-CONH₂ |
| SEQ ID NO: 740 | Alexa Fluor-680-G3-(GfO)3-GPLGIAGITGARGLA-(GPO)3-CONH₂ |
| SEQ ID NO: 741 | Cyanine 3-G3-(GfO)3-GPLGIAGITGARGLA-(GPO)3-CONH₂ |
| SEQ ID NO: 742 | Cyanine 5-G3-(GfO)3-GPLGIAGITGARGLA-(GPO)3-CONH₂ |
| SEQ ID NO: 743 | Cyanine 5.5-G3-(GfO)3-GPLGIAGITGARGLA-(GPO)3-CONH₂ |
| SEQ ID NO: 744 | Cyanine 7-G3-(GfO)3-GPLGIAGITGARGLA-(GPO)3-CONH₂ |
| SEQ ID NO: 745 | Cyanine 7.5-G3-(GfO)3-GPLGIAGITGARGLA-(GPO)3-CONH₂ |
| SEQ ID NO: 746 | Cyanine 3-phycoerythrin-G3-(GfO)3-GPLGIAGITGARGLA-(GPO)3-CONH₂ |
| SEQ ID NO: 747 | Cyanine 5-phycoerythrin-G3-(GfO)3-GPLGIAGITGARGLA-(GPO)3-CONH₂ |
| SEQ ID NO: 748 | Cyanine 5.5-phycoerythrin-G3-(GfO)3-GPLGIAGITGARGLA-(GPO)3-CONH₂ |
| SEQ ID NO: 749 | Cyanine 7-phycoerythrin-G3-(GfO)3-GPLGIAGITGARGLA-(GPO)3-CONH₂ |
| SEQ ID NO: 750 | Cyanine 7.5-phycoerythrin-G3-(GfO)3-GPLGIAGITGARGLA-(GPO)3-CONH₂ |
| SEQ ID NO: 751 | Rhodamine-G3-(GfO)3-GPLGIAGITGARGLA-(GPO)3-CONH₂ |
| SEQ ID NO: 752 | Sulfo-Cyanine3-G3-(GfO)3-GPLGIAGITGARGLA-(GPO)3-CONH₂ |
| SEQ ID NO: 753 | Sulfo-Cyanine5-G3-(GfO)3-GPLGIAGITGARGLA-(GPO)3-CONH₂ |
| SEQ ID NO: 754 | Sulfo-Cyanine5.5-G3-(GfO)3-GPLGIAGITGARGLA-(GPO)3-CONH₂ |
| SEQ ID NO: 755 | Sulfo-Cyanine7-G3-(GfO)3-GPLGIAGITGARGLA-(GPO)3-CONH₂ |
| SEQ ID NO: 756 | Sulfo-Cyanine7.5-G3-(GfO)3-GPLGIAGITGARGLA-(GPO)3-CONH₂ |
| SEQ ID NO: 757 | Texas Red-G3-(GfO)3-GPLGIAGITGARGLA-(GPO)3-CONH₂ |
| SEQ ID NO: 758 | Biotin-G3-(GfO)3-GPLGIAGITGARGLA-(GPO)2-CONH₂ |
| SEQ ID NO: 759 | 5(6)-Carboxyfluorescein-G3-(GfO)3-GPLGIAGITGARGLA-(GPO)2-CONH₂ |
| SEQ ID NO: 760 | Alexa Fluor-350-G3-(GfO)3-GPLGIAGITGARGLA-(GPO)2-CONH₂ |
| SEQ ID NO: 761 | Alexa Fluor-430-G3-(GfO)3-GPLGIAGITGARGLA-(GPO)2-CONH₂ |
| SEQ ID NO: 762 | Alexa Fluor-488-G3-(GfO)3-GPLGIAGITGARGLA-(GPO)2-CONH₂ |
| SEQ ID NO: 763 | Alexa Fluor-546-G3-(GfO)3-GPLGIAGITGARGLA-(GPO)2-CONH₂ |
| SEQ ID NO: 764 | Alexa Fluor-568-G3-(GfO)3-GPLGIAGITGARGLA-(GPO)2-CONH₂ |
| SEQ ID NO: 765 | Alexa Fluor-594-G3-(GfO)3-GPLGIAGITGARGLA-(GPO)2-CONH₂ |
| SEQ ID NO: 766 | Alexa Fluor-633-G3-(GfO)3-GPLGIAGITGARGLA-(GPO)2-CONH₂ |
| SEQ ID NO: 767 | Alexa Fluor-660-G3-(GfO)3-GPLGIAGITGARGLA-(GPO)2-CONH₂ |
| SEQ ID NO: 768 | Alexa Fluor-680-G3-(GfO)3-GPLGIAGITGARGLA-(GPO)2-CONH₂ |
| SEQ ID NO: 769 | Cyanine 3-G3-(GfO)3-GPLGIAGITGARGLA-(GPO)2-CONH₂ |
| SEQ ID NO: 770 | Cyanine 5-G3-(GfO)3-GPLGIAGITGARGLA-(GPO)2-CONH₂ |
| SEQ ID NO: 771 | Cyanine 5.5-G3-(GfO)3-GPLGIAGITGARGLA-(GPO)2-CONH₂ |
| SEQ ID NO: 772 | Cyanine 7-G3-(GfO)3-GPLGIAGITGARGLA-(GPO)2-CONH₂ |
| SEQ ID NO: 773 | Cyanine 7.5-G3-(GfO)3-GPLGIAGITGARGLA-(GPO)2-CONH₂ |
| SEQ ID NO: 774 | Cyanine 3-phycoerythrin-G3-(GfO)3-GPLGIAGITGARGLA-(GPO)2-CONH₂ |
| SEQ ID NO: 775 | Cyanine 5-phycoerythrin-G3-(GfO)3-GPLGIAGITGARGLA-(GPO)2-CONH₂ |
| SEQ ID NO: 776 | Cyanine 5.5-phycoerythrin-G3-(GfO)3-GPLGIAGITGARGLA-(GPO)2-CONH₂ |
| SEQ ID NO: 777 | Cyanine 7-phycoerythrin-G3-(GfO)3-GPLGIAGITGARGLA-(GPO)2-CONH₂ |
| SEQ ID NO: 778 | Cyanine 7.5-phycoerythrin-G3-(GfO)3-GPLGIAGITGARGLA-(GPO)2-CONH₂ |
| SEQ ID NO: 779 | Rhodamine-G3-(GfO)3-GPLGIAGITGARGLA-(GPO)2-CONH₂ |
| SEQ ID NO: 780 | Sulfo-Cyanine3-G3-(GfO)3-GPLGIAGITGARGLA-(GPO)2-CONH₂ |
| SEQ ID NO: 781 | Sulfo-Cyanine5-G3-(GfO)3-GPLGIAGITGARGLA-(GPO)2-CONH₂ |
| SEQ ID NO: 782 | Sulfo-Cyanine5.5-G3-(GfO)3-GPLGIAGITGARGLA-(GPO)2-CONH₂ |
| SEQ ID NO: 783 | Sulfo-Cyanine7-G3-(GfO)3-GPLGIAGITGARGLA-(GPO)2-CONH₂ |
| SEQ ID NO: 784 | Sulfo-Cyanine7.5-G3-(GfO)3-GPLGIAGITGARGLA-(GPO)2-CONH₂ |
| SEQ ID NO: 785 | Texas Red-G3-(GfO)3-GPLGIAGITGARGLA-(GPO)2-CONH₂ |
| SEQ ID NO: 786 | Biotin-G3-(GfO)3-GPLGIAGITGARGLA-(GfO)2-CONH₂ |
| SEQ ID NO: 787 | 5(6)-Carboxyfluorescein-G3-(GfO)3-GPLGIAGITGARGLA-(GfO)2-CONH₂ |
| SEQ ID NO: 788 | Alexa Fluor-350-G3-(GfO)3-GPLGIAGITGARGLA-(GfO)2-CONH₂ |
| SEQ ID NO: 789 | Alexa Fluor-430-G3-(GfO)3-GPLGIAGITGARGLA-(GfO)2-CONH₂ |
| SEQ ID NO: 790 | Alexa Fluor-488-G3-(GfO)3-GPLGIAGITGARGLA-(GfO)2-CONH₂ |
| SEQ ID NO: 791 | Alexa Fluor-546-G3-(GfO)3-GPLGIAGITGARGLA-(GfO)2-CONH₂ |
| SEQ ID NO: 792 | Alexa Fluor-568-G3-(GfO)3-GPLGIAGITGARGLA-(GfO)2-CONH₂ |
| SEQ ID NO: 793 | Alexa Fluor-594-G3-(GfO)3-GPLGIAGITGARGLA-(GfO)2-CONH₂ |
| SEQ ID NO: 794 | Alexa Fluor-633-G3-(GfO)3-GPLGIAGITGARGLA-(GfO)2-CONH₂ |
| SEQ ID NO: 795 | Alexa Fluor-660-G3-(GfO)3-GPLGIAGITGARGLA-(GfO)2-CONH₂ |
| SEQ ID NO: 796 | Alexa Fluor-680-G3-(GfO)3-GPLGIAGITGARGLA-(GfO)2-CONH₂ |
| SEQ ID NO: 797 | Cyanine 3-G3-(GfO)3-GPLGIAGITGARGLA-(GfO)2-CONH₂ |
| SEQ ID NO: 798 | Cyanine 5-G3-(GfO)3-GPLGIAGITGARGLA-(GfO)2-CONH₂ |
| SEQ ID NO: 799 | Cyanine 5.5-G3-(GfO)3-GPLGIAGITGARGLA-(GfO)2-CONH₂ |
| SEQ ID NO: 800 | Cyanine 7-G3-(GfO)3-GPLGIAGITGARGLA-(GfO)2-CONH₂ |
| SEQ ID NO: 801 | Cyanine 7.5-G3-(GfO)3-GPLGIAGITGARGLA-(GfO)2-CONH₂ |
| SEQ ID NO: 802 | Cyanine 3-phycoerythrin-G3-(GfO)3-GPLGIAGITGARGLA-(GfO)2-CONH₂ |
| SEQ ID NO: 803 | Cyanine 5-phycoerythrin-G3-(GfO)3-GPLGIAGITGARGLA-(GfO)2-CONH₂ |
| SEQ ID NO: 804 | Cyanine 5.5-phycoerythrin-G3-(GfO)3-GPLGIAGITGARGLA-(GfO)2-CONH₂ |
| SEQ ID NO: 805 | Cyanine 7-phycoerythrin-G3-(GfO)3-GPLGIAGITGARGLA-(GfO)2-CONH₂ |
| SEQ ID NO: 806 | Cyanine 7.5-phycoerythrin-G3-(GfO)3-GPLGIAGITGARGLA-(GfO)2-CONH₂ |
| SEQ ID NO: 807 | Rhodamine-G3-(GfO)3-GPLGIAGITGARGLA-(GfO)2-CONH₂ |
| SEQ ID NO: 808 | Sulfo-Cyanine3-G3-(GfO)3-GPLGIAGITGARGLA-(GfO)2-CONH₂ |
| SEQ ID NO: 809 | Sulfo-Cyanine5-G3-(GfO)3-GPLGIAGITGARGLA-(GfO)2-CONH₂ |
| SEQ ID NO: 810 | Sulfo-Cyanine5.5-G3-(GfO)3-GPLGIAGITGARGLA-(GfO)2-CONH₂ |
| SEQ ID NO: 811 | Sulfo-Cyanine7-G3-(GfO)3-GPLGIAGITGARGLA-(GfO)2-CONH₂ |
| SEQ ID NO: 812 | Sulfo-Cyanine7.5-G3-(GfO)3-GPLGIAGITGARGLA-(GfO)2-CONH₂ |
| SEQ ID NO: 813 | Texas Red-G3-(GfO)3-GPLGIAGITGARGLA-(GfO)2-CONH₂ |
| SEQ ID NO: 814 | Biotin-G3-(GfO)3-GPLGIAGITGARGLA-(GfO)3-CONH₂ |
| SEQ ID NO: 815 | 5(6)-Carboxyfluorescein-G3-(GfO)3-GPLGIAGITGARGLA-(GfO)3-CONH₂ |
| SEQ ID NO: 816 | Alexa Fluor-350-G3-(GfO)3-GPLGIAGITGARGLA-(GfO)3-CONH₂ |
| SEQ ID NO: 817 | Alexa Fluor-430-G3-(GfO)3-GPLGIAGITGARGLA-(GfO)3-CONH₂ |
| SEQ ID NO: 818 | Alexa Fluor-488-G3-(GfO)3-GPLGIAGITGARGLA-(GfO)3-CONH₂ |
| SEQ ID NO: 819 | Alexa Fluor-546-G3-(GfO)3-GPLGIAGITGARGLA-(GfO)3-CONH₂ |
| SEQ ID NO: 820 | Alexa Fluor-568-G3-(GfO)3-GPLGIAGITGARGLA-(GfO)3-CONH₂ |
| SEQ ID NO: 821 | Alexa Fluor-594-G3-(GfO)3-GPLGIAGITGARGLA-(GfO)3-CONH₂ |
| SEQ ID NO: 822 | Alexa Fluor-633-G3-(GfO)3-GPLGIAGITGARGLA-(GfO)3-CONH₂ |
| SEQ ID NO: 823 | Alexa Fluor-660-G3-(GfO)3-GPLGIAGITGARGLA-(GfO)3-CONH₂ |
| SEQ ID NO: 824 | Alexa Fluor-680-G3-(GfO)3-GPLGIAGITGARGLA-(GfO)3-CONH₂ |
| SEQ ID NO: 825 | Cyanine 3-G3-(GfO)3-GPLGIAGITGARGLA-(GfO)3-CONH₂ |
| SEQ ID NO: 826 | Cyanine 5-G3-(GfO)3-GPLGIAGITGARGLA-(GfO)3-CONH₂ |
| SEQ ID NO: 827 | Cyanine 5.5-G3-(GfO)3-GPLGIAGITGARGLA-(GfO)3-CONH₂ |
| SEQ ID NO: 828 | Cyanine 7-G3-(GfO)3-GPLGIAGITGARGLA-(GfO)3-CONH₂ |
| SEQ ID NO: 829 | Cyanine 7.5-G3-(GfO)3-GPLGIAGITGARGLA-(GfO)3-CONH₂ |
| SEQ ID NO: 830 | Cyanine 3-phycoerythrin-G3-(GfO)3-GPLGIAGITGARGLA-(GfO)3-CONH₂ |
| SEQ ID NO: 831 | Cyanine 5-phycoerythrin-G3-(GfO)3-GPLGIAGITGARGLA-(GfO)3-CONH₂ |
| SEQ ID NO: 832 | Cyanine 5.5-phycoerythrin-G3-(GfO)3-GPLGIAGITGARGLA-(GfO)3-CONH₂ |
| SEQ ID NO: 833 | Cyanine 7-phycoerythrin-G3-(GfO)3-GPLGIAGITGARGLA-(GfO)3-CONH₂ |
| SEQ ID NO: 834 | Cyanine 7.5-phycoerythrin-G3-(GfO)3-GPLGIAGITGARGLA-(GfO)3-CONH₂ |
| SEQ ID NO: 835 | Rhodamine-G3-(GfO)3-GPLGIAGITGARGLA-(GfO)3-CONH₂ |
| SEQ ID NO: 836 | Sulfo-Cyanine3-G3-(GfO)3-GPLGIAGITGARGLA-(GfO)3-CONH₂ |
| SEQ ID NO: 837 | Sulfo-Cyanine5-G3-(GfO)3-GPLGIAGITGARGLA-(GfO)3-CONH₂ |
| SEQ ID NO: 838 | Sulfo-Cyanine5.5-G3-(GfO)3-GPLGIAGITGARGLA-(GfO)3-CONH₂ |
| SEQ ID NO: 839 | Sulfo-Cyanine7-G3-(GfO)3-GPLGIAGITGARGLA-(GfO)3-CONH₂ |
| SEQ ID NO: 840 | Sulfo-Cyanine7.5-G3-(GfO)3-GPLGIAGITGARGLA-(GfO)3-CONH₂ |
| SEQ ID NO: 841 | Texas Red-G3-(GfO)3-GPLGIAGITGARGLA-(GfO)3-CONH₂ |
| SEQ ID NO: 842 | Biotin-G3-(GPO)3-GPQGLLGAG-(GPO)2-CONH₂ |
| SEQ ID NO: 843 | 5(6)-Carboxyfluorescein-G3-(GPO)3-GPQGLLGAG-(GPO)2-CONH₂ |
| SEQ ID NO: 844 | Alexa Fluor-350-G3-(GPO)3-GPQGLLGAG-(GPO)2-CONH₂ |
| SEQ ID NO: 845 | Alexa Fluor-430-G3-(GPO)3-GPQGLLGAG-(GPO)2-CONH₂ |
| SEQ ID NO: 846 | Alexa Fluor-488-G3-(GPO)3-GPQGLLGAG-(GPO)2-CONH₂ |
| SEQ ID NO: 847 | Alexa Fluor-546-G3-(GPO)3-GPQGLLGAG-(GPO)2-CONH₂ |
| SEQ ID NO: 848 | Alexa Fluor-568-G3-(GPO)3-GPQGLLGAG-(GPO)2-CONH₂ |
| SEQ ID NO: 849 | Alexa Fluor-594-G3-(GPO)3-GPQGLLGAG-(GPO)2-CONH₂ |
| SEQ ID NO: 850 | Alexa Fluor-633-G3-(GPO)3-GPQGLLGAG-(GPO)2-CONH₂ |
| SEQ ID NO: 851 | Alexa Fluor-660-G3-(GPO)3-GPQGLLGAG-(GPO)2-CONH₂ |
| SEQ ID NO: 852 | Alexa Fluor-680-G3-(GPO)3-GPQGLLGAG-(GPO)2-CONH₂ |
| SEQ ID NO: 853 | Cyanine 3-G3-(GPO)3-GPQGLLGAG-(GPO)2-CONH₂ |
| SEQ ID NO: 854 | Cyanine 5-G3-(GPO)3-GPQGLLGAG-(GPO)2-CONH₂ |
| SEQ ID NO: 855 | Cyanine 5.5-G3-(GPO)3-GPQGLLGAG-(GPO)2-CONH₂ |
| SEQ ID NO: 856 | Cyanine 7-G3-(GPO)3-GPQGLLGAG-(GPO)2-CONH₂ |
| SEQ ID NO: 857 | Cyanine 7.5-G3-(GPO)3-GPQGLLGAG-(GPO)2-CONH₂ |
| SEQ ID NO: 858 | Cyanine 3-phycoerythrin-G3-(GPO)3-GPQGLLGAG-(GPO)2-CONH₂ |
| SEQ ID NO: 859 | Cyanine 5-phycoerythrin-G3-(GPO)3-GPQGLLGAG-(GPO)2-CONH₂ |
| SEQ ID NO: 860 | Cyanine 5.5-phycoerythrin-G3-(GPO)3-GPQGLLGAG-(GPO)2-CONH₂ |
| SEQ ID NO: 861 | Cyanine 7-phycoerythrin-G3-(GPO)3-GPQGLLGAG-(GPO)2-CONH₂ |
| SEQ ID NO: 862 | Cyanine 7.5-phycoerythrin-G3-(GPO)3-GPQGLLGAG-(GPO)2-CONH₂ |
| SEQ ID NO: 863 | Rhodamine-G3-(GPO)3-GPQGLLGAG-(GPO)2-CONH₂ |
| SEQ ID NO: 864 | Sulfo-Cyanine3-G3-(GPO)3-GPQGLLGAG-(GPO)2-CONH₂ |
| SEQ ID NO: 865 | Sulfo-Cyanine5-G3-(GPO)3-GPQGLLGAG-(GPO)2-CONH₂ |
| SEQ ID NO: 866 | Sulfo-Cyanine5.5-G3-(GPO)3-GPQGLLGAG-(GPO)2-CONH₂ |
| SEQ ID NO: 867 | Sulfo-Cyanine7-G3-(GPO)3-GPQGLLGAG-(GPO)2-CONH₂ |
| SEQ ID NO: 868 | Sulfo-Cyanine7.5-G3-(GPO)3-GPQGLLGAG-(GPO)2-CONH₂ |
| SEQ ID NO: 869 | Texas Red-G3-(GPO)3-GPQGLLGAG-(GPO)2-CONH₂ |
| SEQ ID NO: 870 | Biotin-G3-(GPO)3-GPQGLLGAG-(GPO)3-CONH₂ |
| SEQ ID NO: 871 | 5(6)-Carboxyfluorescein-G3-(GPO)3-GPQGLLGAG-(GPO)3-CONH₂ |
| SEQ ID NO: 872 | Alexa Fluor-350-G3-(GPO)3-GPQGLLGAG-(GPO)3-CONH₂ |
| SEQ ID NO: 873 | Alexa Fluor-430-G3-(GPO)3-GPQGLLGAG-(GPO)3-CONH₂ |
| SEQ ID NO: 874 | Alexa Fluor-488-G3-(GPO)3-GPQGLLGAG-(GPO)3-CONH₂ |
| SEQ ID NO: 875 | Alexa Fluor-546-G3-(GPO)3-GPQGLLGAG-(GPO)3-CONH₂ |
| SEQ ID NO: 876 | Alexa Fluor-568-G3-(GPO)3-GPQGLLGAG-(GPO)3-CONH₂ |
| SEQ ID NO: 877 | Alexa Fluor-594-G3-(GPO)3-GPQGLLGAG-(GPO)3-CONH₂ |
| SEQ ID NO: 878 | Alexa Fluor-633-G3-(GPO)3-GPQGLLGAG-(GPO)3-CONH₂ |
| SEQ ID NO: 879 | Alexa Fluor-660-G3-(GPO)3-GPQGLLGAG-(GPO)3-CONH₂ |
| SEQ ID NO: 880 | Alexa Fluor-680-G3-(GPO)3-GPQGLLGAG-(GPO)3-CONH₂ |
| SEQ ID NO: 881 | Cyanine 3-G3-(GPO)3-GPQGLLGAG-(GPO)3-CONH₂ |
| SEQ ID NO: 882 | Cyanine 5-G3-(GPO)3-GPQGLLGAG-(GPO)3-CONH₂ |
| SEQ ID NO: 883 | Cyanine 5.5-G3-(GPO)3-GPQGLLGAG-(GPO)3-CONH₂ |
| SEQ ID NO: 884 | Cyanine 7-G3-(GPO)3-GPQGLLGAG-(GPO)3-CONH₂ |
| SEQ ID NO: 885 | Cyanine 7.5-G3-(GPO)3-GPQGLLGAG-(GPO)3-CONH₂ |
| SEQ ID NO: 886 | Cyanine 3-phycoerythrin-G3-(GPO)3-GPQGLLGAG-(GPO)3-CONH₂ |
| SEQ ID NO: 887 | Cyanine 5-phycoerythrin-G3-(GPO)3-GPQGLLGAG-(GPO)3-CONH₂ |
| SEQ ID NO: 888 | Cyanine 5.5-phycoerythrin-G3-(GPO)3-GPQGLLGAG-(GPO)3-CONH₂ |
| SEQ ID NO: 889 | Cyanine 7-phycoerythrin-G3-(GPO)3-GPQGLLGAG-(GPO)3-CONH₂ |
| SEQ ID NO: 890 | Cyanine 7.5-phycoerythrin-G3-(GPO)3-GPQGLLGAG-(GPO)3-CONH₂ |
| SEQ ID NO: 891 | Rhodamine-G3-(GPO)3-GPQGLLGAG-(GPO)3-CONH₂ |
| SEQ ID NO: 892 | Sulfo-Cyanine3-G3-(GPO)3-GPQGLLGAG-(GPO)3-CONH₂ |
| SEQ ID NO: 893 | Sulfo-Cyanine5-G3-(GPO)3-GPQGLLGAG-(GPO)3-CONH₂ |
| SEQ ID NO: 894 | Sulfo-Cyanine5.5-G3-(GPO)3-GPQGLLGAG-(GPO)3-CONH₂ |
| SEQ ID NO: 895 | Sulfo-Cyanine7-G3-(GPO)3-GPQGLLGAG-(GPO)3-CONH₂ |
| SEQ ID NO: 896 | Sulfo-Cyanine7.5-G3-(GPO)3-GPQGLLGAG-(GPO)3-CONH₂ |
| SEQ ID NO: 897 | Texas Red-G3-(GPO)3-GPQGLLGAG-(GPO)3-CONH₂ |
| SEQ ID NO: 898 | Biotin-G3-(GfO)3-GPQGLLGAG-(GPO)3-CONH₂ |
| SEQ ID NO: 899 | 5(6)-Carboxyfluorescein-G3-(GfO)3-GPQGLLGAG-(GPO)3-CONH₂ |
| SEQ ID NO: 900 | Alexa Fluor-350-G3-(GfO)3-GPQGLLGAG-(GPO)3-CONH₂ |
| SEQ ID NO: 901 | Alexa Fluor-430-G3-(GfO)3-GPQGLLGAG-(GPO)3-CONH₂ |
| SEQ ID NO: 902 | Alexa Fluor-488-G3-(GfO)3-GPQGLLGAG-(GPO)3-CONH₂ |
| SEQ ID NO: 903 | Alexa Fluor-546-G3-(GfO)3-GPQGLLGAG-(GPO)3-CONH₂ |
| SEQ ID NO: 904 | Alexa Fluor-568-G3-(GfO)3-GPQGLLGAG-(GPO)3-CONH₂ |
| SEQ ID NO: 905 | Alexa Fluor-594-G3-(GfO)3-GPQGLLGAG-(GPO)3-CONH₂ |
| SEQ ID NO: 906 | Alexa Fluor-633-G3-(GfO)3-GPQGLLGAG-(GPO)3-CONH₂ |
| SEQ ID NO: 907 | Alexa Fluor-660-G3-(GfO)3-GPQGLLGAG-(GPO)3-CONH₂ |
| SEQ ID NO: 908 | Alexa Fluor-680-G3-(GfO)3-GPQGLLGAG-(GPO)3-CONH₂ |
| SEQ ID NO: 909 | Cyanine 3-G3-(GfO)3-GPQGLLGAG-(GPO)3-CONH₂ |
| SEQ ID NO: 910 | Cyanine 5-G3-(GfO)3-GPQGLLGAG-(GPO)3-CONH₂ |
| SEQ ID NO: 911 | Cyanine 5.5-G3-(GfO)3-GPQGLLGAG-(GPO)3-CONH₂ |
| SEQ ID NO: 912 | Cyanine 7-G3-(GfO)3-GPQGLLGAG-(GPO)3-CONH₂ |
| SEQ ID NO: 913 | Cyanine 7.5-G3-(GfO)3-GPQGLLGAG-(GPO)3-CONH₂ |
| SEQ ID NO: 914 | Cyanine 3-phycoerythrin-G3-(GfO)3-GPQGLLGAG-(GPO)3-CONH₂ |
| SEQ ID NO: 915 | Cyanine 5-phycoerythrin-G3-(GfO)3-GPQGLLGAG-(GPO)3-CONH₂ |
| SEQ ID NO: 916 | Cyanine 5.5-phycoerythrin-G3-(GfO)3-GPQGLLGAG-(GPO)3-CONH₂ |
| SEQ ID NO: 917 | Cyanine 7-phycoerythrin-G3-(GfO)3-GPQGLLGAG-(GPO)3-CONH₂ |
| SEQ ID NO: 918 | Cyanine 7.5-phycoerythrin-G3-(GfO)3-GPQGLLGAG-(GPO)3-CONH₂ |
| SEQ ID NO: 919 | Rhodamine-G3-(GfO)3-GPQGLLGAG-(GPO)3-CONH₂ |
| SEQ ID NO: 920 | Sulfo-Cyanine3-G3-(GfO)3-GPQGLLGAG-(GPO)3-CONH₂ |
| SEQ ID NO: 921 | Sulfo-Cyanine5-G3-(GfO)3-GPQGLLGAG-(GPO)3-CONH₂ |
| SEQ ID NO: 922 | Sulfo-Cyanine5.5-G3-(GfO)3-GPQGLLGAG-(GPO)3-CONH₂ |
| SEQ ID NO: 923 | Sulfo-Cyanine7-G3-(GfO)3-GPQGLLGAG-(GPO)3-CONH₂ |
| SEQ ID NO: 924 | Sulfo-Cyanine7.5-G3-(GfO)3-GPQGLLGAG-(GPO)3-CONH₂ |
| SEQ ID NO: 925 | Texas Red-G3-(GfO)3-GPQGLLGAG-(GPO)3-CONH₂ |
| SEQ ID NO: 926 | Biotin-G3-(GfO)3-GPQGLLGAG-(GPO)2-CONH₂ |
| SEQ ID NO: 927 | 5(6)-Carboxyfluorescein-G3-(GfO)3-GPQGLLGAG-(GPO)2-CONH₂ |
| SEQ ID NO: 928 | Alexa Fluor-350-G3-(GfO)3-GPQGLLGAG-(GPO)2-CONH₂ |
| SEQ ID NO: 929 | Alexa Fluor-430-G3-(GfO)3-GPQGLLGAG-(GPO)2-CONH₂ |
| SEQ ID NO: 930 | Alexa Fluor-488-G3-(GfO)3-GPQGLLGAG-(GPO)2-CONH₂ |
| SEQ ID NO: 931 | Alexa Fluor-546-G3-(GfO)3-GPQGLLGAG-(GPO)2-CONH₂ |
| SEQ ID NO: 932 | Alexa Fluor-568-G3-(GfO)3-GPQGLLGAG-(GPO)2-CONH₂ |
| SEQ ID NO: 933 | Alexa Fluor-594-G3-(GfO)3-GPQGLLGAG-(GPO)2-CONH₂ |
| SEQ ID NO: 934 | Alexa Fluor-633-G3-(GfO)3-GPQGLLGAG-(GPO)2-CONH₂ |
| SEQ ID NO: 935 | Alexa Fluor-660-G3-(GfO)3-GPQGLLGAG-(GPO)2-CONH₂ |
| SEQ ID NO: 936 | Alexa Fluor-680-G3-(GfO)3-GPQGLLGAG-(GPO)2-CONH₂ |
| SEQ ID NO: 937 | Cyanine 3-G3-(GfO)3-GPQGLLGAG-(GPO)2-CONH₂ |
| SEQ ID NO: 938 | Cyanine 5-G3-(GfO)3-GPQGLLGAG-(GPO)2-CONH₂ |
| SEQ ID NO: 939 | Cyanine 5.5-G3-(GfO)3-GPQGLLGAG-(GPO)2-CONH₂ |
| SEQ ID NO: 940 | Cyanine 7-G3-(GfO)3-GPQGLLGAG-(GPO)2-CONH₂ |
| SEQ ID NO: 941 | Cyanine 7.5-G3-(GfO)3-GPQGLLGAG-(GPO)2-CONH₂ |
| SEQ ID NO: 942 | Cyanine 3-phycoerythrin-G3-(GfO)3-GPQGLLGAG-(GPO)2-CONH₂ |
| SEQ ID NO: 943 | Cyanine 5-phycoerythrin-G3-(GfO)3-GPQGLLGAG-(GPO)2-CONH₂ |
| SEQ ID NO: 944 | Cyanine 5.5-phycoerythrin-G3-(GfO)3-GPQGLLGAG-(GPO)2-CONH₂ |
| SEQ ID NO: 945 | Cyanine 7-phycoerythrin-G3-(GfO)3-GPQGLLGAG-(GPO)2-CONH₂ |
| SEQ ID NO: 946 | Cyanine 7.5-phycoerythrin-G3-(GfO)3-GPQGLLGAG-(GPO)2-CONH₂ |
| SEQ ID NO: 947 | Rhodamine-G3-(GfO)3-GPQGLLGAG-(GPO)2-CONH₂ |
| SEQ ID NO: 948 | Sulfo-Cyanine3-G3-(GfO)3-GPQGLLGAG-(GPO)2-CONH₂ |
| SEQ ID NO: 949 | Sulfo-Cyanine5-G3-(GfO)3-GPQGLLGAG-(GPO)2-CONH₂ |
| SEQ ID NO: 950 | Sulfo-Cyanine5.5-G3-(GfO)3-GPQGLLGAG-(GPO)2-CONH₂ |
| SEQ ID NO: 951 | Sulfo-Cyanine7-G3-(GfO)3-GPQGLLGAG-(GPO)2-CONH₂ |
| SEQ ID NO: 952 | Sulfo-Cyanine7.5-G3-(GfO)3-GPQGLLGAG-(GPO)2-CONH₂ |
| SEQ ID NO: 953 | Texas Red-G3-(GfO)3-GPQGLLGAG-(GPO)2-CONH₂ |
| SEQ ID NO: 954 | Biotin-G3-(GfO)3-GPQGLLGAG-(GfO)2-CONH₂ |
| SEQ ID NO: 955 | 5(6)-Carboxyfluorescein-G3-(GfO)3-GPQGLLGAG-(GfO)2-CONH₂ |
| SEQ ID NO: 956 | Alexa Fluor-350-G3-(GfO)3-GPQGLLGAG-(GfO)2-CONH₂ |
| SEQ ID NO: 957 | Alexa Fluor-430-G3-(GfO)3-GPQGLLGAG-(GfO)2-CONH₂ |
| SEQ ID NO: 958 | Alexa Fluor-488-G3-(GfO)3-GPQGLLGAG-(GfO)2-CONH₂ |
| SEQ ID NO: 959 | Alexa Fluor-546-G3-(GfO)3-GPQGLLGAG-(GfO)2-CONH₂ |
| SEQ ID NO: 960 | Alexa Fluor-568-G3-(GfO)3-GPQGLLGAG-(GfO)2-CONH₂ |
| SEQ ID NO: 961 | Alexa Fluor-594-G3-(GfO)3-GPQGLLGAG-(GfO)2-CONH₂ |
| SEQ ID NO: 962 | Alexa Fluor-633-G3-(GfO)3-GPQGLLGAG-(GfO)2-CONH₂ |
| SEQ ID NO: 963 | Alexa Fluor-660-G3-(GfO)3-GPQGLLGAG-(GfO)2-CONH₂ |
| SEQ ID NO: 964 | Alexa Fluor-680-G3-(GfO)3-GPQGLLGAG-(GfO)2-CONH₂ |
| SEQ ID NO: 965 | Cyanine 3-G3-(GfO)3-GPQGLLGAG-(GfO)2-CONH₂ |
| SEQ ID NO: 966 | Cyanine 5-G3-(GfO)3-GPQGLLGAG-(GfO)2-CONH₂ |
| SEQ ID NO: 967 | Cyanine 5.5-G3-(GfO)3-GPQGLLGAG-(GfO)2-CONH₂ |
| SEQ ID NO: 968 | Cyanine 7-G3-(GfO)3-GPQGLLGAG-(GfO)2-CONH₂ |
| SEQ ID NO: 969 | Cyanine 7.5-G3-(GfO)3-GPQGLLGAG-(GfO)2-CONH₂ |
| SEQ ID NO: 970 | Cyanine 3-phycoerythrin-G3-(GfO)3-GPQGLLGAG-(GfO)2-CONH₂ |
| SEQ ID NO: 971 | Cyanine 5-phycoerythrin-G3-(GfO)3-GPQGLLGAG-(GfO)2-CONH₂ |
| SEQ ID NO: 972 | Cyanine 5.5-phycoerythrin-G3-(GfO)3-GPQGLLGAG-(GfO)2-CONH₂ |
| SEQ ID NO: 973 | Cyanine 7-phycoerythrin-G3-(GfO)3-GPQGLLGAG-(GfO)2-CONH₂ |
| SEQ ID NO: 974 | Cyanine 7.5-phycoerythrin-G3-(GfO)3-GPQGLLGAG-(GfO)2-CONH₂ |
| SEQ ID NO: 975 | Rhodamine-G3-(GfO)3-GPQGLLGAG-(GfO)2-CONH₂ |
| SEQ ID NO: 976 | Sulfo-Cyanine3-G3-(GfO)3-GPQGLLGAG-(GfO)2-CONH₂ |
| SEQ ID NO: 977 | Sulfo-Cyanine5-G3-(GfO)3-GPQGLLGAG-(GfO)2-CONH₂ |
| SEQ ID NO: 978 | Sulfo-Cyanine5.5-G3-(GfO)3-GPQGLLGAG-(GfO)2-CONH₂ |
| SEQ ID NO: 979 | Sulfo-Cyanine7-G3-(GfO)3-GPQGLLGAG-(GfO)2-CONH₂ |
| SEQ ID NO: 980 | Sulfo-Cyanine7.5-G3-(GfO)3-GPQGLLGAG-(GfO)2-CONH₂ |
| SEQ ID NO: 981 | Texas Red-G3-(GfO)3-GPQGLLGAG-(GfO)2-CONH₂ |
| SEQ ID NO: 982 | Biotin-G3-(GfO)3-GPQGLLGAG-(GfO)3-CONH₂ |
| SEQ ID NO: 983 | 5(6)-Carboxyfluorescein-G3-(GfO)3-GPQGLLGAG-(GfO)3-CONH₂ |
| SEQ ID NO: 984 | Alexa Fluor-350-G3-(GfO)3-GPQGLLGAG-(GfO)3-CONH₂ |
| SEQ ID NO: 985 | Alexa Fluor-430-G3-(GfO)3-GPQGLLGAG-(GfO)3-CONH₂ |
| SEQ ID NO: 986 | Alexa Fluor-488-G3-(GfO)3-GPQGLLGAG-(GfO)3-CONH₂ |
| SEQ ID NO: 987 | Alexa Fluor-546-G3-(GfO)3-GPQGLLGAG-(GfO)3-CONH₂ |
| SEQ ID NO: 988 | Alexa Fluor-568-G3-(GfO)3-GPQGLLGAG-(GfO)3-CONH₂ |
| SEQ ID NO: 989 | Alexa Fluor-594-G3-(GfO)3-GPQGLLGAG-(GfO)3-CONH₂ |
| SEQ ID NO: 990 | Alexa Fluor-633-G3-(GfO)3-GPQGLLGAG-(GfO)3-CONH₂ |
| SEQ ID NO: 991 | Alexa Fluor-660-G3-(GfO)3-GPQGLLGAG-(GfO)3-CONH₂ |
| SEQ ID NO: 992 | Alexa Fluor-680-G3-(GfO)3-GPQGLLGAG-(GfO)3-CONH₂ |
| SEQ ID NO: 993 | Cyanine 3-G3-(GfO)3-GPQGLLGAG-(GfO)3-CONH₂ |
| SEQ ID NO: 994 | Cyanine 5-G3-(GfO)3-GPQGLLGAG-(GfO)3-CONH₂ |
| SEQ ID NO: 995 | Cyanine 5.5-G3-(GfO)3-GPQGLLGAG-(GfO)3-CONH₂ |
| SEQ ID NO: 996 | Cyanine 7-G3-(GfO)3-GPQGLLGAG-(GfO)3-CONH₂ |
| SEQ ID NO: 997 | Cyanine 7.5-G3-(GfO)3-GPQGLLGAG-(GfO)3-CONH₂ |
| SEQ ID NO: 998 | Cyanine 3-phycoerythrin-G3-(GfO)3-GPQGLLGAG-(GfO)3-CONH₂ |
| SEQ ID NO: 999 | Cyanine 5-phycoerythrin-G3-(GfO)3-GPQGLLGAG-(GfO)3-CONH₂ |
| SEQ ID NO: 1000 | Cyanine 5.5-phycoerythrin-G3-(GfO)3-GPQGLLGAG-(GfO)3-CONH₂ |
| SEQ ID NO: 1001 | Cyanine 7-phycoerythrin-G3-(GfO)3-GPQGLLGAG-(GfO)3-CONH₂ |
| SEQ ID NO: 1002 | Cyanine 7.5-phycoerythrin-G3-(GfO)3-GPQGLLGAG-(GfO)3-CONH₂ |
| SEQ ID NO: 1003 | Rhodamine-G3-(GfO)3-GPQGLLGAG-(GfO)3-CONH₂ |
| SEQ ID NO: 1004 | Sulfo-Cyanine3-G3-(GfO)3-GPQGLLGAG-(GfO)3-CONH₂ |
| SEQ ID NO: 1005 | Sulfo-Cyanine5-G3-(GfO)3-GPQGLLGAG-(GfO)3-CONH₂ |
| SEQ ID NO: 1006 | Sulfo-Cyanine5.5-G3-(GfO)3-GPQGLLGAG-(GfO)3-CONH₂ |
| SEQ ID NO: 1007 | Sulfo-Cyanine7-G3-(GfO)3-GPQGLLGAG-(GfO)3-CONH₂ |
| SEQ ID NO: 1008 | Sulfo-Cyanine7.5-G3-(GfO)3-GPQGLLGAG-(GfO)3-CONH₂ |

In certain sequences provided in Table 1 above, ‘GGG’ or “G3” represents a Triple Glycine spacer. In certain sequences provided in Table 1 above, ‘NH₂’ represents an amidated C-terminus. In certain sequences provided in Table 1 above, the ‘f’ in a ‘GfO’ sequence represents a 2S, 4S-4-fluoroproline (cis conformation). In certain sequences provided in Table 1 above, ‘Ahx’ represents a 6-aminohexanoic acid spacer.

In some embodiments, the detection moiety of each peptide in Table 1 may be replaced with another one or more detection moiety described herein. In some embodiments, the spacer moiety of each peptide in Table 1 may be replaced with another spacer moiety disclosed herein. In some embodiments, the terminus moiety of LCHP described herein may include any one of the terminus moieties in Table 1.

In some embodiments, the CHP (Gly-X-Y)ₙ repeating portion has a sequence selected from Table 2 below.

TABLE 2
Sequences for Repeating Portion of Collagen Hybridizing Peptides
Sequence (One-Letter Amino Acid Symbols)

(GPO)₃₋₂₀, where G = glycine, P = proline, O = trans-hydroxyproline
(2S,4R-hydroxyproline)
(GfO)₃₋₂₀, where G = glycine, f = cis-fluoroproline (2S,4S-4-
fluoroproline), O = trans-hydroxyproline (2S,4R-hydroxyproline)
(GcO)₃₋₂₀, where G = glycine, c = cis-chloroproline (2S,4S-4-
chloroproline), O = trans-hydroxyproline (2S,4R-hydroxyproline)
(GOP)₃₋₂₀, where G = glycine, P = proline, O = trans-hydroxyproline
(2S,4R-hydroxyproline)
(_{az}GPO)₃₋₂₀, where _{az}G = aza-glycine, P = proline, O = trans-
hydroxyproline (2S,4R-hydroxyproline)
(_{az}GfO)₃₋₂₀, where _{az}G = aza-glycine, f = cis-fluoroproline (2S,4S-4-
fluoroproline), O = trans-hydroxyproline (2S,4R-hydroxyproline)

In some embodiments, the LCHP described herein may comprise two (Gly-X-Y)n repeating portions. In some embodiments, the LCHP described herein may comprise one or more (Gly-X-Y)n repeating portion and one or more (Gly-X-Y)q repeating portion, wherein q is any integer from 0 to 25. In some embodiments, n may be 3. In some embodiments, q may be 2. In some embodiments, q may be 3.

In some embodiments, the LCHP comprises a SEQ ID NO: 55.

Other Embodiments and Equivalents including, but not limited to, a dimeric version of each sequence listed in Tables 1 and 2. In certain embodiments, a dimeric sequence can differ from an amino acid sequence as provided in any of Tables 1 and 2 by 1 amino acid, 2 amino acids, 3 amino acids, 4 amino acids 5 amino acids, 6 amino acids, 7 amino acids, 8 amino acids, 9 amino acids, 10 amino acids or greater than 10 amino acids. In certain embodiments, a dimeric sequence can comprise a glycine offset and/or a lysine branch point.

LCHPs bind to the degrading collagen via a unique thermodynamically driven binding mechanism where the CHPs fold into a collagen triple helix with the available denatured collagen alpha strands. This may allow for the detection of denatured/remodeled collagen strands located in the tissue.

In some embodiments, the LCHPs are administered to the subject by topical administration. In some embodiments, the LCHPs are administered to the subject by local injection. In some embodiments, the LCHPs are administered to the subject by intravenous injection.

In an exemplary embodiment, the LCHP forms a triple helix with native collagen alpha-strands in the subject.

In some example embodiments, certain amino acids in the CHP sequence can serve as cleavage sites for serum proteins while maintaining high triple helix propensity. In some embodiments, the CHP may have a MMP cleavable sequence which can serve as cleavage sites for MMPs in serum and extracellular matrix while maintaining high triple helix propensity. In some embodiments, the CHP described herein may comprise charged residues that are recognized by enzymes. For example, the CHP described herein may comprise lysine that is recognized by trypsin and other enzymes. In some embodiments, the CHPs produced are short while maintaining high triple helix propensity.

IIIb. Imaging In Vivo

In an exemplary embodiment, the LCHPs are imaged in-vivo. In an exemplary embodiment, the imaging in-vivo comprises angiography. In an exemplary embodiment, the imaging in-vivo is angiography. In an exemplary embodiment, the imaging in-vivo comprises optical coherence tomography (OCT). In an exemplary embodiment, the imaging in-vivo is optical coherence tomography (OCT).

In some embodiments, the LCHPs are imaged on an eye of the subject. In some embodiments, the imaging is performed within 2, 2.5, 3, 3.5, 4, 4.5, 5 or 5.5 hours from administering the LCHPs. In some embodiments, the imaging is performed within about 0.2, 0.5, 1, 1.5, 2, 2.5, 3, 3.5, 4, 4.5, 5, 5.5, 6, 7, 8, 9, or 10 hours from administering the LCHPs. In some embodiments, the imaging is performed within about 24, 23, 22, 21, 20, 19, 18, 17, 15, 14, 13, 12, 11, 10, 9, 8, 7, 6, 5, 4, 3, or 2 hours from administering the LCHPs. In some embodiments, the imaging is performed within about 5, 4, 3, 2, or 1 day(s) from administering the LCHPs.

Immunofluorescent microscopy can be used to image detection moieties (such as the ALEXA FLUOR dyes, cyanine dyes, sulfo-cyanine dyes, indocyanine dyes, TIDE FLUOR dyes, TAMRA, FITC, 5-FAM, carboxyfluorescein, coumarin dyes, and rhodamine dyes) on tissues (EVOS M5000 with the correct light cubes). For in vivo work these detection moieties can be imaged by three-dimensional (3D) fluorescence molecular tomographic imaging (FMT imaging) or Near-Infrared fluorescence imaging (Perkin Elmer IVIS spectrum for example).

Magnetic resonance imaging (MRI), optical imaging, OCT, or computed tomography (CT), can be used for imaging when gold nanoparticles are the detection moiety. Magnetic resonance imaging (MRI) can be used for imaging when iron oxide nanoparticles are the detection moiety. Positron emission tomography (PET) can be used for imaging when a radiolabel is the detection moiety.

### IV. Progression of Fibrosis

In one aspect, the present disclosure provides a method of detecting fibrosis progression in a subject, comprising administering a labeled collagen hybridizing peptide (LCHP) to the subject, and imaging the LCHPs in-vivo, further comprising administering another LCHP to the subject at another time point, imaging said another LCHP in-vivo, and comparing images from the different time points, thereby detecting the progression of fibrosis in the subject. In an exemplary embodiment, the fibrosis is subretinal fibrosis.

### V. Diagnosis and Treatment of Fibrotic Diseases

In an exemplary embodiment, the invention provides a method of diagnosing a fibrotic disease in a subject based on the presence or progression of fibrosis in the subject detected by a method described herein. In an exemplary embodiment, the fibrotic disease is a fibrotic eye disease. In an exemplary embodiment, the fibrotic disease is selected from the group consisting of neovascular age-related macular degeneration (nAMD), diabetic retinopathy, glaucoma specifically fibrosis in the trabecular meshwork, neovascular glaucoma, corneal scarring, conjunctiva, post cataract surgery, retinopathy of prematurity, and proliferative vitreoretinopathy. In an exemplary embodiment, the fibrotic disease is nAMD. In an exemplary embodiment, the fibrotic disease is glaucoma including fibrosis in trabecular meshwork.

In an exemplary embodiment, the invention provides a method of treating a subject with a fibrotic disease, comprising diagnosing a fibrotic disease in a subject in accordance with a method described herein, and administering an antifibrotic drug to the subject.

In an exemplary embodiment, the invention provides a method of treating a subject with neovascular age-related macular degeneration (nAMD, comprising diagnosing nAMD in a subject in accordance with a method described herein, and administering an antifibrotic drug to the subject

In some embodiments, the antifibrotic drug may comprise nintedanib and/or pirfenidone. In some embodiments, the antifibrotic drug may comprise an anti-vascular endothelial growth factor (VEGF) agent.

In an exemplary embodiment, the fibrotic disease is non-alcoholic steatohepatitis (NASH), non-alcoholic fatty liver disease (NAFLD), keloids, chronic kidney disease (CKD), bone marrow fibrosis, idiopathic pulmonary fibrosis (IPF), and age-related macular degeneration (AMD), such as neovascular age-related macular degeneration (nAMD).

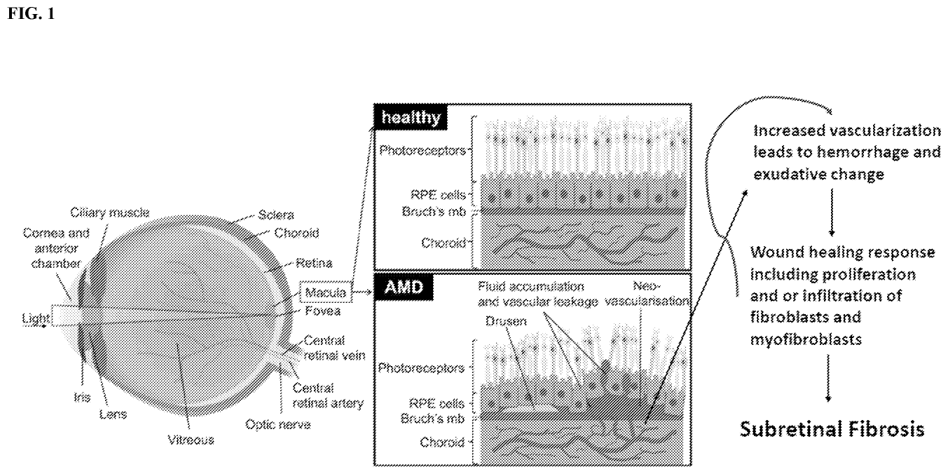

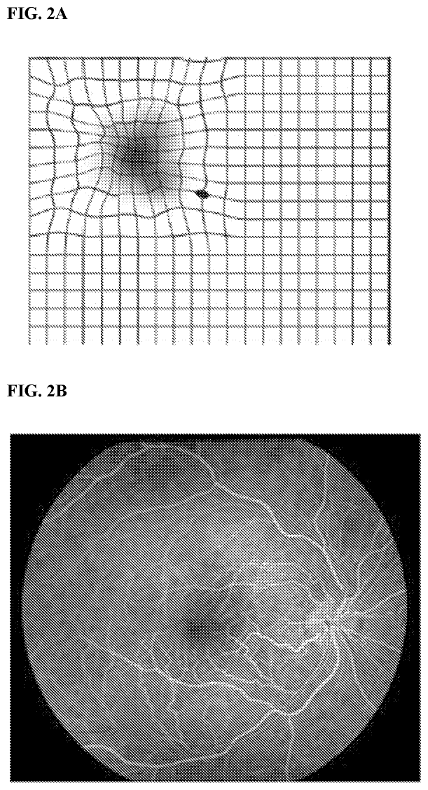

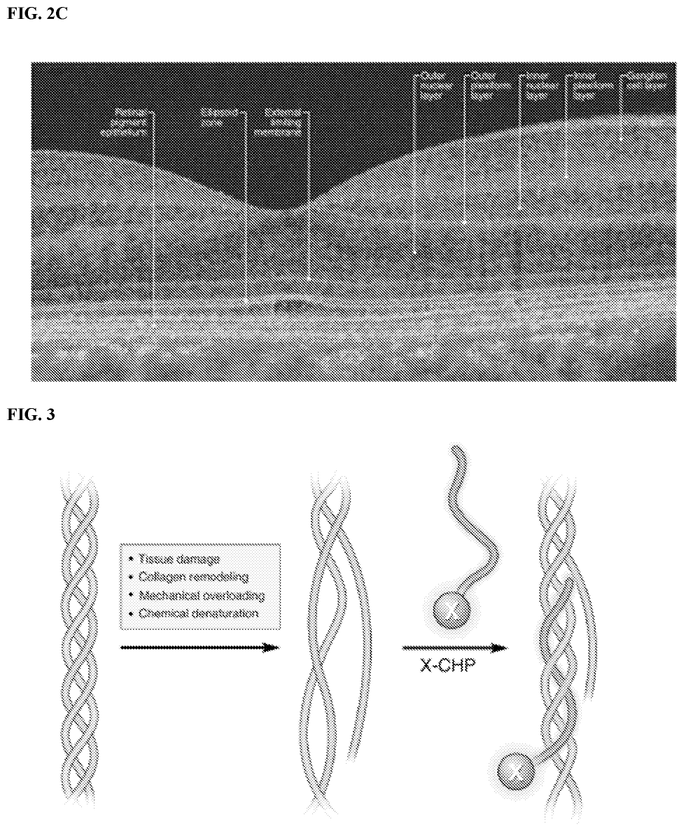

There are two types of AMD. Dry AMD is the most common form and experienced by about 80% of people who have the dry form of AMD. Dry AMD occurs when parts of the macula get thinner with age and clumps of protein (i.e., drusen) grow (not illustrated). Wet AMD is less common but often more serious in which abnormal blood vessels grow under the retina (as illustrated in FIG. 1). The vessels under the retina may leak blood of other fluids which causes scarring of the macula. Specifically, the blood vessels grow from the choroid across Bruch's membrane and into the RPE cells. Increased vascularization of the blood vessels may also lead to hemorrhage and exudative change. In the process of wound healing which includes proliferation and/or infiltration of fibroblasts and micro fibroblasts leads to subretinal fibrosis.

Anti-VEGF treatments can slow the vascularization associated with nAMD, resulting in improved vision in many of the patients. However, as many as 40% of patients develop subretinal-fibrosis after 10 years of treatment with an anti-VEGF treatment for nAMD. Subretinal fibrosis is directly associated to the loss of vision. Current diagnostic tools lack the ability to monitor the fibrotic progression, especially in the early stages.

In some embodiments, the subject may not have previously been diagnosed with fibrosis. In some embodiments, the subject may not have previously been diagnosed with nAMD.

In some embodiments, the subject may have previously been diagnosed with fibrosis. The subject may optionally have already undergone treatment for fibrosis or the one or more complications related to fibrosis.

In some embodiments, the subject may have previously been diagnosed with nAMD. The subject may optionally have already undergone treatment for nAMD or the one or more complications related to nAMD.

In some embodiments, a method of treating a subject with neovascular age-related macular degeneration (nAMD) includes diagnosing nAMD in a subject as described herein. In some embodiments, a method of treating a subject with neovascular age-related macular degeneration (nAMD) includes administering an antifibrotic drug to the subject.

### VI. Additional Analyses

In some embodiments, the method described herein may further comprise analyzing any one of triple helical stability and propensity, affinity to denatured collagen by using crosslinked gelatin, mechanically damaged tendon, tissue sections (heart, lung, muscle, bone, kidney, liver, etc.), serum stability (e.g., HPLC, MS), circular dichroism (CD), and overall size (e.g., dynamic light scattering).

In some embodiments, in the method described herein, the CHP's biocompatibility is analyzed relating to the fibrotic tissue remodeling process and systemic toxicity, as well as means to remove bound CHP by cellular activity. The analysis may be conducted by using nAMD model mouse and investigate the effects of CHP binding on nAMD healing response using histology. In some embodiments, the analysis may include conducting complete blood count in a subject after multiple CHP dosage.

### EXAMPLES

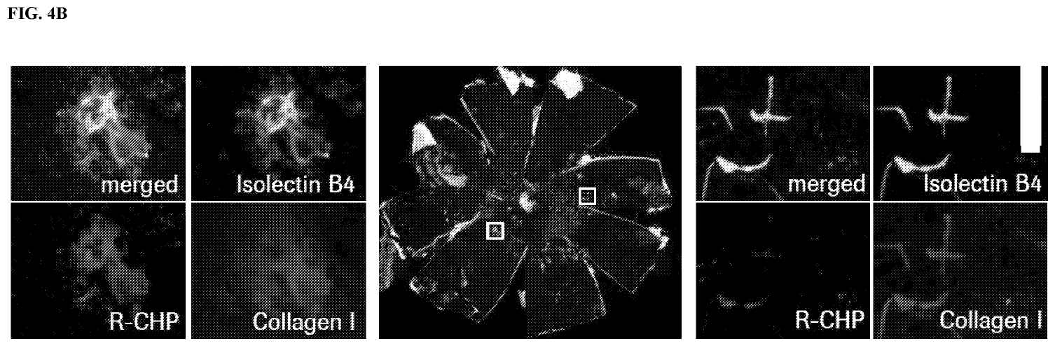

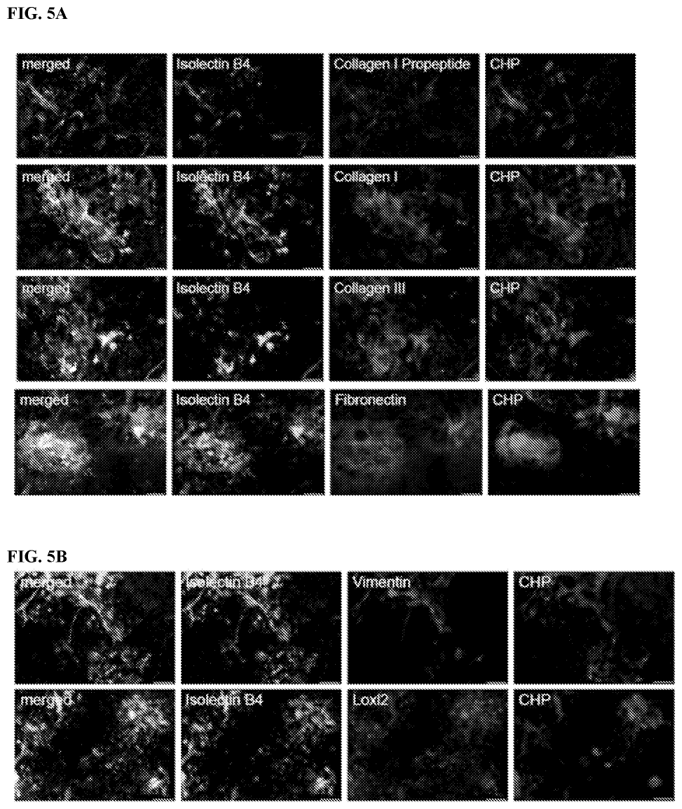

### Example 1: Retinal Pigment Epithelium (RPE)/Choroid Flat-Mount Staining FIGS. 4 & 5

Sulfo-Cy3-G₃-(GPO)₉ (SEQ ID NO: 23) (sCy3-conjugated CHP) was synthesized using solid phase peptide synthesis, for example, as described in U.S. Patent Application Publication No. 2017/0112940, which is incorporated by reference in its entirety.

Two mouse models were implemented for evaluating the damaged and denatured collagen in nAMD. A spontaneous choroidal neovascularization (CNV) model utilized the JR5558 mouse model derived from the C57BL/6J parent strain purchased from Charles River (Germany) at 4-5 weeks of age (Nagai et al. Investig. Ophthalmol. Vis. Sci. 2014, 55 (6), 3709-3719). In the spontaneous CNV model, vascularization started early and originated in the choroid between postnatal day 10 and 15. Neovascularization increased in number and severity causing disruption and dysfunction in the retinal pigment epithelium (RPE). This was verified using fluorescein angiography and fundus angiography imaging. The other model for inducing neovascularization to mimic nAMD was a laser-induced neovascularization mouse model. C57BL/6J mice, purchased from Charles River (France) at 10-12 weeks of age, were anesthetized, had pupils dilated, and a Phoenix Micron IV retinal imaging microscope (Phoenix Research Labs) coupled to a Meridian Merilas 532α green laser (Thun, Switzerland) was used to place 4 lesions around the optic nerve of each eye with an intensity of 150, 300, 400 or 500 mW (100 ms).

The mice were sacrificed by cervical dislocation under isoflurane anesthesia. Eyes were harvested and immediately fixed in 4% PFA for 2 h at RT. The RPE/choroid was separated from the retina and permeabilized for 2 h in 3% Triton X-100 solution in PBS. After permeabilization, the RPE/choroids were stained in 48-well plates (1×RPE/choroid flat-mount per well in 200 ul volume).

The sCy3-conjugated CHP (conc.: 2 uM, RED300, 3Helix) in 1×PBS were heated for 5 minutes at 80° C. and afterwards quenched using ice-cold water for 30-60 seconds. 100 ul of the CHPs solution were added per RPE/choroid flat-mount. For ab co-stainings, Fibronectin (1:100, ab23750, abcam) and Isolectin B4 (1:100, L2140, Sigma) antibodies (in 100 ul 1×PBS per RPE/choroid FM), were added to the RPE/choroid flat-mounts and incubated overnight at 4° C. The next day, the flat mounts were washed 5×5 min with 1×PBS and afterwards incubated for 2 h at RT with DyLight-488 conjugated (1:100, #SA-5488, Vector Laboratories) and Donkey anti-Rabbit IgG (1:200, Alexa Fluor 647, #A31573, Life Technologies) secondary antibody in 1×PBS. Afterward, RPE/choroid tissue was washed 5×5 in 1×PBS at RT, before being mounted with the RPE up on Superfrost glass slides using Dako fluorescent mounting medium (#S3032, Dako). Images were acquired with an Olympus VS-ASW scanner equipped with a XM10 camera (Olympus Soft ImagingSolution software).

FIG. 4A presents flat-mount staining results from the laser-induced choroidal neovascularization (LCNV) model. CHPs (red) allow us to distinguish active fibrotic lesions caused from laser damage or enzymatic turnover from healthy, collagen-rich tissues. Healthy, intact collagen I is stained purple in the images, while the green channel represents fibronectin, Initial histological assessments of CHP binding to regions of collagen turnover nAMD tissue from two mouse models laser induced choroidal neovascularization (CNV) and Spontaneous CNV (JR5558) showed CHPs can effectively bind and visualize the fibrotic material in the tissue sections.

FIG. 4B illustrate an RPE/Choroid flat mount from the spontaneous CNV (JR5558) mouse model (Female mouse, 52 days old). CHPs (red) allow us to distinguish active fibrotic lesions caused from laser damage or enzymatic turnover from healthy, collagen-rich tissues. Healthy, intact collagen I is stained purple in the images, while the green channel represents fibronectin in FIG. 4A and isolectin B4 in FIG. 4B, This figure includes a secondary ROI on the center flatmount image and shows in the boxes on the right-hand side were images taken around newly formed blood vessels. This shows that the CHPs do not bind to the blood vessels unlike isolectin B4 (a common stain for vessels) and collagen I, but CHPs stain the remodeling fibrotic tissue.

The result shows the CHPs bind different areas than the Col I AB but it is a collagen rich area, indicating that this is not a healthy tissue. The red staining from the CHPs shows where areas of high collagen turnover are. Such areas are caused by being damaged with a laser in the LCNV model (FIG. 4A) and caused by the spontaneous remodeling by enzymes in FIG. 4B.

Initial histological assessments of CHP binding to regions of collagen turnover nAMD tissue from two mouse models laser induced choroidal neovascularization (CNV) and Spontaneous CNV (JR5558) showed CHPs can effectively bind and visualize the fibrotic material in the tissue sections.

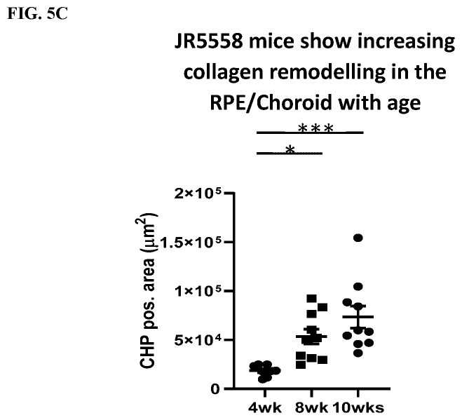

FIGS. 5A and 5B illustrate that CHPs co-localize with fibrotic products such as collagen I pro-peptides, Col I and III, fibronectin as well as endothelial mesenchymal transition (EMT) associated proteins, vimentin and Loxl2. CHP in vivo imaging was performed in JR5558 mice that spontaneously develop fibrotic choroidal neovascularization (CNV) and in male C57BL/6J wild-type mice with laser-induced CNV (LCNV) lesions. JR5558 mice were analyzed at 9-10 weeks of age, LCNV mice were analyzed at 2-4 weeks after the laser injury.

The left most column (“Merged”) in FIG. 5A shows all the channels of that row together in a single image. As moving across each row from left to right, the green channel shows the staining for isolectin B4 with is an indicator of new vasculature. nAMD has aberrant vasculature pushing into the macula causing pressure and loss of vision. Column 3 (purple channel) shows a variety of markers used to stain for fibrosis associated proteins. Pro-collagen I peptide (top row) indicates newly synthesized collagen by identifying the pro-peptides that are cleaved from the N-terminus before collagen is exported. This is a known marker for collagen synthesis. The antibodies for collagen I (row 2) and collagen III (row 3) were used as these collagens are the fibrillar collagen types that get produced in fibrotic conditions. The Fibronectin stain (row 4) shows the staining of increased fibronectin in the area which is another known fibrosis stain. Column 4 (red channel) shows the LCHP staining the damaged, denatured, or remodeling collagen in the area due the remodeling caused by fibrosis. This Image shows how CHP staining compares with common fibrosis proteins that are stained and how LCHPs give different information.

The left most column (“Merged”) in FIG. 5B shows all the channels of that row together in a single image. As moving across each row from left to right, column 2 (green channel) shows the staining for isolectin B4 with is an indicator of new vasculature. Column 3 (purple channel) highlights the common proteins that are stained for to visualize epithelial mesenchymal transition (EMT). EMT is a process for an epithelial cell to undergo a conversion to a mesenchymal phenotype and is an inflammation-induced response, which is involved in fibrotic progression. Vimentin is commonly stained as a marker for mesenchymal cells as it stains an intermediate filament found in these cells. Loxl2 (lysl oxidases-like protein 2) is another stain for EMT in diseases. In column 4 (red channel), CHPS again stain for damaged and denatured collagen.

The graphic in FIG. 5C shows that as nAMD progresses in the JR5558 mice over time, the fibrotic lesions get more severe. This CHP signal was quantified from the average CHP positive areas in RPE/Choroid flat-mount sections taken after 4, 8, and 10 weeks after disease initiation. As time increases, CHP signal also increased.

### Example 2: LCHP In Vivo Imaging

sCy7.5-conjugated CHPs (SEQ ID NO: 55) was synthesized using solid phase peptide synthesis.

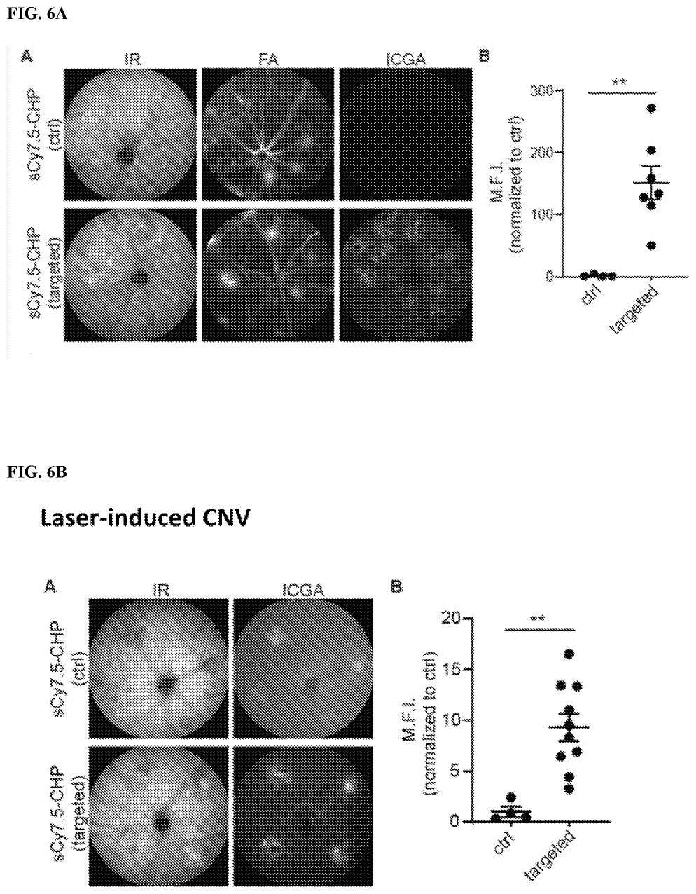

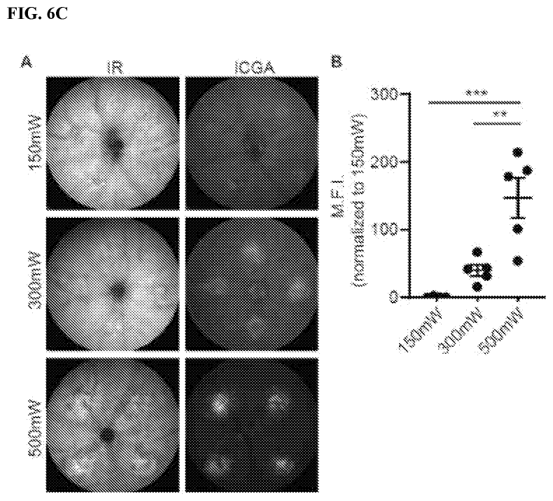

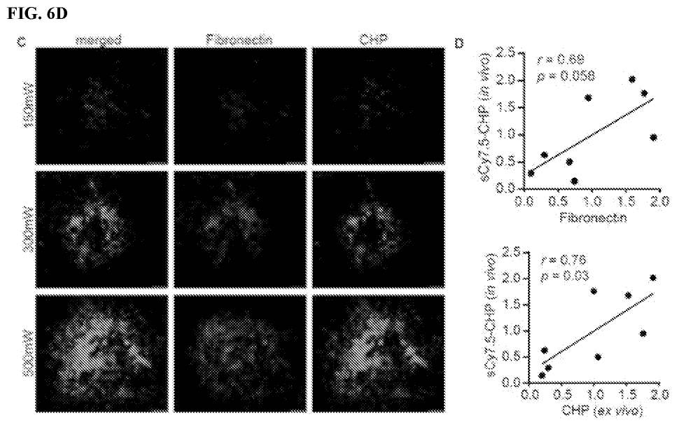

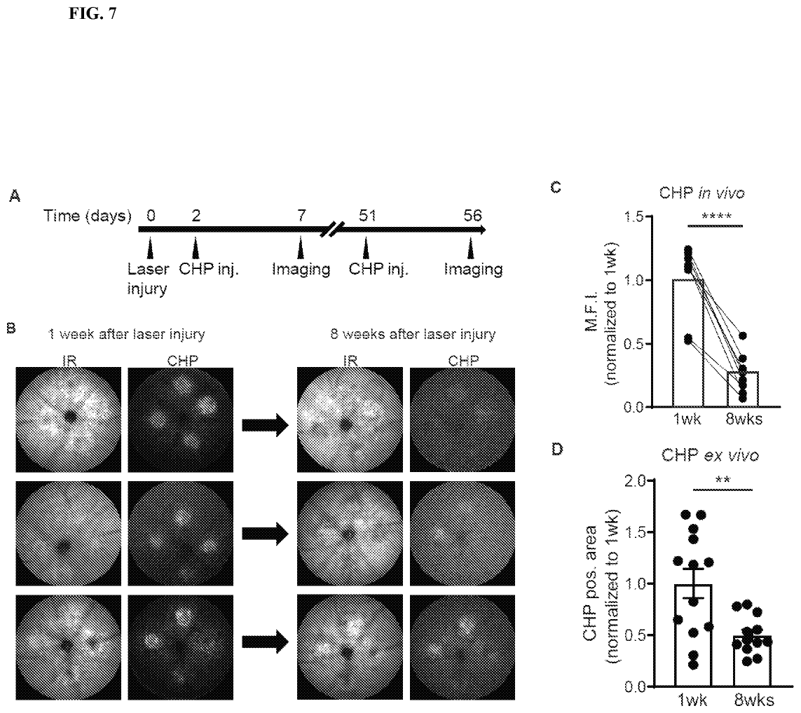

The sCy7.5-conjugated CHP in vivo imaging was performed in JR5558 mice that spontaneously develop fibrotic choroidal neovascularization (CNV) Nagai et al. Investig. Ophthalmol. Vis. Sci. 2014, 55 (6), 3709-3719; and in male C57BL/6J wild-type mice with laser-induced CNV (LCNV) lesions Lambert, V. et al. Nat Protoc 8, 2197-2211, (2013). JR5558 mice were analyzed at 9-10 weeks of age, LCNV mice were analyzed at 2-4 weeks after the laser injury as shown in FIGS. 6A, 6B, and 7.

The sCy7.5-conjugated CHP was injected via the tail vein without anesthesia at a final concentration of 1 nmol per animal (200 ul of 5 uM CHPs in 1×PBS). 5 days after injection, mice were anesthetized with subcutaneous injection of an anesthesia mixture containing fentanyl (0.05 mg/kg), medetomidine (0.5 mg/kg), and midazolam (5 mg/kg). Eyes were dilated with 1% tropicamide to obtain fundus and ICG angiography images on a Heidelberg Spectralis microscope (Heidelberg Engineering).

To show the potential for an in vivo imaging probe, both the JR5558 and Laser Induced CNV models were injected (tail vein injection) with a sCy7.5 CHP probe and control scrambled sequence CHP probe (sulfo-Cy7.5-GGG-OfGGOfGfGfOfOGOfGOOfGGOOff) without anesthesia at a final concentration of 1 nmol per animal (200 ul of 5 uM CHPs in 1×PBS).

Five days after the injection of CHPs, anesthetized with subcutaneous injection of an anesthesia mixture containing fentanyl (0.05 mg/kg), medetomidine (0.5 mg/kg), and midazolam (5 mg/kg). Eyes were dilated with 1% tropicamide to obtain fundus and indocyanine green (ICG) angiography images on a Heidelberg Spectralis microscope (Heidelberg Engineering). The animals were imaged via ICGA (FIGS. 6A-6B, 7, and 8A-8B), which illustrate in vivo imaging using CHPs with OCT for spontaneous CNV and laser-induced CNV.

The results showed an increased binding of targeted sCy7.5-CHP to fibrotic areas compared to the scrambled control sequence. Furthermore, when varying the laser intensity during the laser induced CNV, there was an increased sCy7.5-CHP signal with the higher laser power suggesting that the CHP is able to distinguish severity of the fibrosis.

In the in vivo imaging experiments shown in FIGS. 6A-6B, 7, 8A-8B, the time between CHP injection and imaging of the fibrotic scar was three to five days.

Top Row of FIG. 6A shows the results using the scrambled sCy7.5-CHP control group. The results indicate that in the infrared (IR) channel, no significant signal was detected from the control, also for the fundus angiography (FA) column and the ICGA column. When compared to the targeted sCy7.5-CHP (btm row), higher signal intensity was detected from these imaging techniques. The bright spots were lesions caused by the spontaneous CNV, the sCy7.5-CHPs localized in these active lesions. The active lesions were areas with higher than normal collagen turnover. This confirmed that the CHP was localizing the dye in the areas of interest and it was not due to non-specific binding of the dye and/or peptide sequence used. Panel B showed the mean fluorescent intensity (MFI) normalized to the control signal which confirmed quantitatively that there was increased signal intensity from the sCy7.5-CHP over the scrambled control.

The signal from fibrotic tissue remained for more than a week. The signal remaining for more than a week makes the current design of the CHP molecule unusable in a clinical setting. The main reason for delayed imaging is the low clearance rate of CHP from the systemic circulation which interfere with target signal. The systemic circulation time can be dramatically reduced by changing the structure of the CHP as previously demonstrated (Molecular Pharmaceutics, 2017). The new CHP structures will also help CHP removal from fibrotic tissue after binding. The development of biocompatible fluorescent CHPs which could be used for detecting fibrosis associated with nAMD in clinical setting is therefore crucial.

These results above with regard to FIGS. 6A-6B were also confirmed in the histological section of FIGS. 6C-6D.

The in vivo imaging results in FIG. 7 were obtained from the LCNV mouse model showing decreased collagen remodeling in stabilized vs fresh wounds. The schematic seen at the top describes the timeline for inducing laser injury, CHP injections, as well as imaging (FIG. 7A). Visually, there is a distinct difference in the targeted sCy7.5-CHP signal seen in the mice at 1 week post laser injury vs at 8 weeks post injury (FIG. 7B). When this signal was quantified by normalizing CHP signal to week 1 (FIGS. 7C & 7D), there was a significant decrease in the level of CHP binding, indicating that the lesion was no longer undergoing active remodeling and had reached a stabilized state. This result was corroborated by CHP staining ex vivo as well.

### Example 3: Bispecific Antibody Testing—In Vivo Imaging

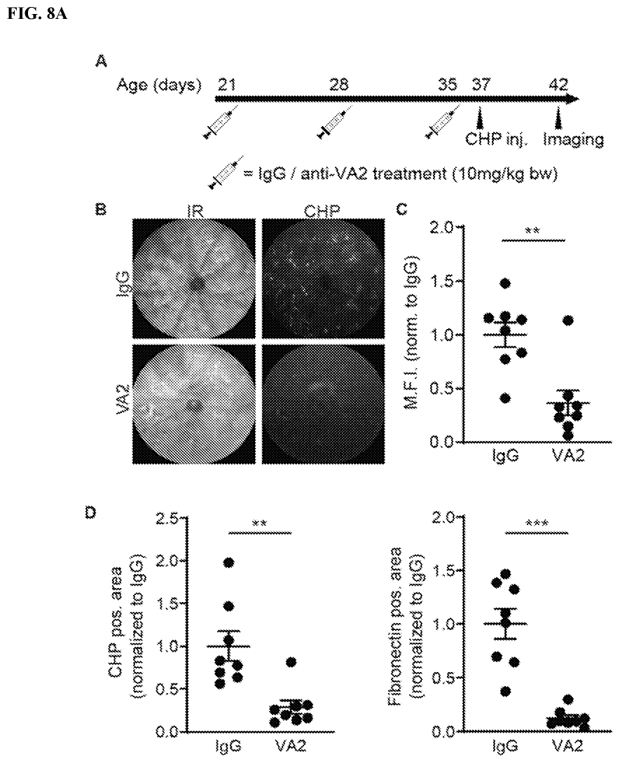

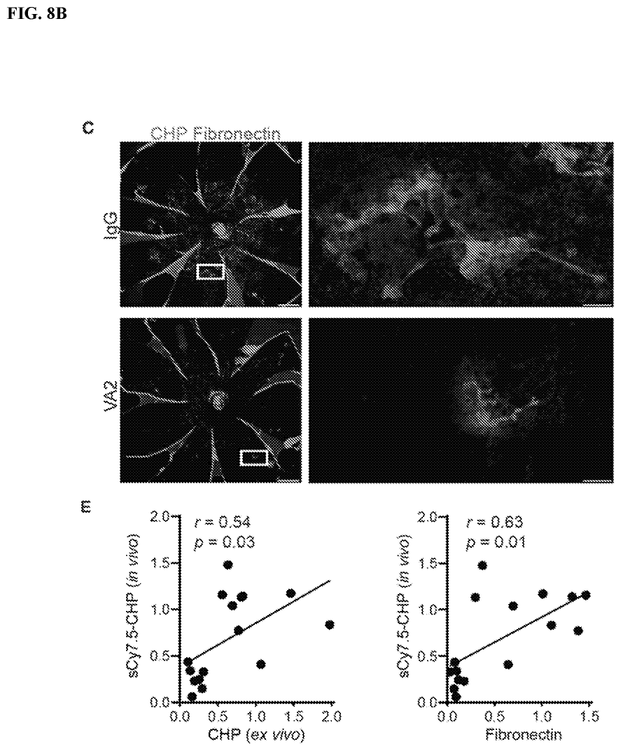

The anti-fibrotic effects of a bispecific angiopoietin-2 (Ang-2)/VEGF antibody (VA2) were examined in vivo by assessing CHP binding in 42-day-old JR5558 mice after 3 VA2 injections (10 mg/kg on days 21, 28 and 35; sCy7.5-CHP injected on day 37) shown in FIGS. 8A-8B.

Validation studies assessed the correlation between CHP and EMT markers, laser intensity and CHP binding in vivo, and in vivo and ex vivo CHP quantification of active collagen

FIG. 8A illustrates how CHPs enabled the monitoring of reduced fibrosis following treatment with a bispecific anti-VEGF/Anti-Ang-2 (VA2) antibody in JR5558 mice. The schematic at the top shows the experimental timeline for VA2 injections, CHP injections, and imaging. Representative in vivo images compare the retinas of mice treated with a common IgG antibody vs mice treated with VA2. sCy7.5-CHPs were used to visualize the damage using a scanning laser ophthalmoscope (cSLO). Mice treated with VA2 had less CHP signal and thus had less fibrosis than the mice treated with IgG. This result was quantified in the graph on the right where the sCy7.5-CHP signal was normalized to IgG, showing a statistically significant decrease in CHP signal in the VA2 treated mice. These results were confirmed by ex vivo staining using R-CHP and fibronectin staining which are shown in the graphs on the bottom. Again, VA2 treated mice showed less fibrotic turnover, evidenced by lower CHP signal compared to the IgG treated mice. This result was corroborated by the fibronectin staining showing a significant decrease of fibronectin in VA2 treated mice.

FIG. 8B Highlights the representative ex vivo staining which utilized R-CHP (red) and a fibronectin stain (purple) that were quantified in the bottom graphs shown in FIG. 8A. Additionally, the correlation between the in vivo and ex vivo CHP signal quantification was r=0.54 and the in vivo CHP signal had a correlation of r=0.63 when compared to the ex vivo fibronectin stain.

In response to the results found in FIGS. 6A-6B, 7, 8A-8B new CHPs were developed that can bind to fibrotic conditions and exhibit a short half-life in vivo. Three different CHP designs were used.

### Example 4: CHP Histology Involving Non-Human Primates

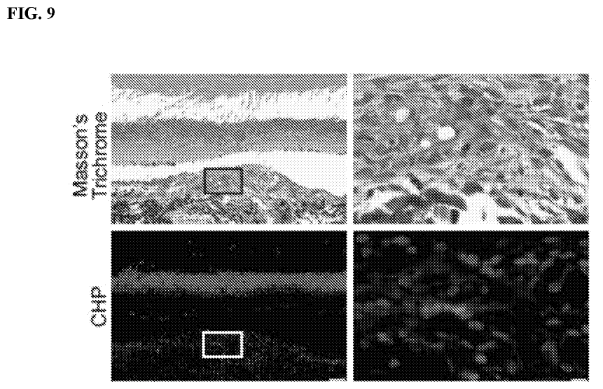

FIG. 9 illustrates CHP histology in non-human primates after laser induced with CNV. Specifically, when CHPs were used to stain histological sections of a non-human primate nAMD model (cynomolgus monkeys), there was positive CHP signal in the fibrotic area.

Experimentation on non-human primates (Macaca fascicularis) was performed in accordance with the Statement for the Use of Animals in Ophthalmic and Vision Research approved by the Association for Research in Vision and Ophthalmology. The guidelines of the Animal Ethics Committee of the Singhealth Singapore Association for Assessment and Accreditation of Laboratory Animal Care were also satisfied. Female cynomolgus monkeys at 3-5 kg body weight were used. CNV was induced by laser photocoagulation on DO in both eyes using a 532-nm laser (PurePoint 532 nm Green Laser; Alcon) attached to a slit-lamp delivery system and a hand-held contact lens. Nine lesions were symmetrically placed in the macula of each eye by a masked retinal specialist. The parameters used were spot size (50 μm), duration (0.1 s), and 500 mW-1 W. The distance from each laser spot to the central fovea was maintained at 0.5-1 disk diameter size. On D30 after LCNV, the animals were sacrificed and the upper body was perfused with half-strength Karnovsky's fixative. The eyes were removed, postfixed for 2-3 days in half-strength Karnovsky's fixative. Strips of tissue containing one or two lesion sites were embedded in plastic. Sections 2-μm thick were taken at 30-μm steps through the middle of each lesion.

In conducting the above tests, it was determined that reducing CHP signal from circulation allows imaging of fibrotic condition at earlier timepoint. Additionally, degradation of CHP by serum protease will reduce CHP's in vivo circulation time.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of detecting fibrosis in a subject, comprising
administering a labeled collagen hybridizing peptide (LCHP) to the subject, and
imaging the LCHPs in-vivo, thereby detecting presence or progression of fibrosis in the subject.

2. The method according to claim 1, wherein the fibrosis is subretinal fibrosis.

3. The method according to claim 1, wherein the LCHP is imaged on an eye of the subject.

4. The method according to claim 1, wherein the LCHP is administered to the subject by topical administration, local injection, or intravenous injection.

5. (canceled)

6. The method according to claim 1, wherein the method detects the progression of fibrosis and further comprises administering another LCHP to the subject at another time point, imaging said another LCHP in-vivo, and comparing images from different time points.

7. The method according to claim 1, wherein the imaging comprises angiography.

8. The method according to claim 1, wherein the imaging comprises optical coherence tomography (OCT).

9. The method according to claim 1, wherein the imaging is performed within 5 days from administering the LCHP.

10. (canceled)

11. (canceled)

12. (canceled)

13. The method according to claim 1, wherein the LCHP comprises
a sequence represented by Formula I:
L-S-(Gly-X-Y)a-b Formula I
in which L is one or more detection moieties; S is zero or more spacer molecules; Gly is glycine; at least one of X and Y is proline, modified proline, and/or hydroxyproline; and a is 3 and b is 20.

14. The method according to claim 1, wherein the LCHP comprises
a sequence represented by Formula II:
L-S-(Gly-X-Y)n-(Gly-A-B)p-(Gly-X-Y)_{q} Formula II
in which L is one or more detection moieties; S is zero or more spacer molecules; Gly is glycine; at least one of X and Y is proline, modified proline, and/or hydroxyproline; each of A and B are independently an amino acid, n is an integer from 1 to 20, p is an integer from 1 to 20, and q is an integer from 1 to 20.

15. The method according to claim 13, wherein said one or more detection moieties
comprise a dye configured to be detected at a wavelength from 340 nm to 800 nm.

16. (canceled)

17. (canceled)

18. The method according to claim 13, wherein said one or more detection
moieties comprise a gold particle.

19. The method according to claim 13, wherein said one or more detection
moieties comprise a label selected from the group consisting of prednisolone acetate, triamcinolone acetonide, and lipid-based artificial tears.

20. The method according to claim 1, wherein at least one of the LCHP comprises the amino acid sequence of any one of SEQ ID NOs: 1-1009.

21. (canceled)

22. (canceled)

23. A method of diagnosing a fibrotic disease in a subject based on the presence or progression of fibrosis in the subject detected by the method of claim 1.

24. The method according to claim 23, wherein the fibrotic disease is a fibrotic eye disease.

25. The method according to claim 23, wherein the fibrosis is subretinal fibrosis.

26. The method according to claim 23, wherein the fibrotic disease is selected from the group consisting of neovascular age-related macular degeneration (nAMD), diabetic retinopathy, glaucoma specifically fibrosis in the trabecular meshwork, neovascular glaucoma, corneal scarring, conjunctiva, post cataract surgery, retinopathy of prematurity, and proliferative vitreoretinopathy.

27. (canceled)

28. (canceled)

29. A method of treating a subject with a fibrotic disease, comprising
diagnosing a fibrotic disease in a subject in accordance with the method of claim 1, and
administering an antifibrotic drug to the subject.

30. A method of treating a subject with neovascular age-related macular degeneration (nAMD), comprising
diagnosing nAMD in a subject in accordance with the method of claim 23,
and administering an antifibrotic drug to the subject.


SEQUENCE LISTING (1009 sequences)

SEQ ID NO: 1 - protein, 30 aa, synthetic construct
  MOD_RES 1: note = Biotin
  MOD_RES 30: note = CONH2
GGGGPOGPOGPOGPOGPOGPOGPOGPOGPO

SEQ ID NO: 2 - protein, 30 aa, synthetic construct; identical to SEQ ID NO: 1

SEQ ID NO: 3 - protein, 30 aa, synthetic construct; identical to SEQ ID NO: 1

SEQ ID NO: 4 - protein, 30 aa, synthetic construct; identical to SEQ ID NO: 1

SEQ ID NO: 5 - protein, 30 aa, synthetic construct; identical to SEQ ID NO: 1

SEQ ID NO: 6 - protein, 30 aa, synthetic construct; identical to SEQ ID NO: 1

SEQ ID NO: 7 - protein, 30 aa, synthetic construct; identical to SEQ ID NO: 1

SEQ ID NO: 8 - protein, 30 aa, synthetic construct; identical to SEQ ID NO: 1

SEQ ID NO: 9 - protein, 30 aa, synthetic construct; identical to SEQ ID NO: 1

SEQ ID NO: 10 - protein, 30 aa, synthetic construct; identical to SEQ ID NO: 1

SEQ ID NO: 11 - protein, 30 aa, synthetic construct; identical to SEQ ID NO: 1

SEQ ID NO: 12 - protein, 30 aa, synthetic construct; identical to SEQ ID NO: 1

SEQ ID NO: 13 - protein, 30 aa, synthetic construct; identical to SEQ ID NO: 1

SEQ ID NO: 14 - protein, 30 aa, synthetic construct; identical to SEQ ID NO: 1

SEQ ID NO: 15 - protein, 30 aa, synthetic construct; identical to SEQ ID NO: 1

SEQ ID NO: 16 - protein, 30 aa, synthetic construct; identical to SEQ ID NO: 1

SEQ ID NO: 17 - protein, 30 aa, synthetic construct; identical to SEQ ID NO: 1

SEQ ID NO: 18 - protein, 30 aa, synthetic construct; identical to SEQ ID NO: 1

SEQ ID NO: 19 - protein, 30 aa, synthetic construct; identical to SEQ ID NO: 1

SEQ ID NO: 20 - protein, 30 aa, synthetic construct; identical to SEQ ID NO: 1

SEQ ID NO: 21 - protein, 30 aa, synthetic construct; identical to SEQ ID NO: 1

SEQ ID NO: 22 - protein, 30 aa, synthetic construct; identical to SEQ ID NO: 1

SEQ ID NO: 23 - protein, 30 aa, synthetic construct; identical to SEQ ID NO: 1

SEQ ID NO: 24 - protein, 30 aa, synthetic construct; identical to SEQ ID NO: 1

SEQ ID NO: 25 - protein, 30 aa, synthetic construct; identical to SEQ ID NO: 1

SEQ ID NO: 26 - protein, 30 aa, synthetic construct; identical to SEQ ID NO: 1

SEQ ID NO: 27 - protein, 30 aa, synthetic construct; identical to SEQ ID NO: 1

SEQ ID NO: 28 - protein, 30 aa, synthetic construct; identical to SEQ ID NO: 1

SEQ ID NO: 29 - protein, 30 aa, synthetic construct
GGGGFOGFOGFOGFOGFOGFOGFOGFOGFO

SEQ ID NO: 30 - protein, 30 aa, synthetic construct; identical to SEQ ID NO: 29

SEQ ID NO: 31 - protein, 30 aa, synthetic construct; identical to SEQ ID NO: 29

SEQ ID NO: 32 - protein, 30 aa, synthetic construct; identical to SEQ ID NO: 29

SEQ ID NO: 33 - protein, 30 aa, synthetic construct; identical to SEQ ID NO: 29

SEQ ID NO: 34 - protein, 30 aa, synthetic construct; identical to SEQ ID NO: 29

SEQ ID NO: 35 - protein, 30 aa, synthetic construct; identical to SEQ ID NO: 29

SEQ ID NO: 36 - protein, 30 aa, synthetic construct; identical to SEQ ID NO: 29

SEQ ID NO: 37 - protein, 30 aa, synthetic construct; identical to SEQ ID NO: 29

SEQ ID NO: 38 - protein, 30 aa, synthetic construct; identical to SEQ ID NO: 29

SEQ ID NO: 39 - protein, 30 aa, synthetic construct; identical to SEQ ID NO: 29

SEQ ID NO: 40 - protein, 30 aa, synthetic construct; identical to SEQ ID NO: 29

SEQ ID NO: 41 - protein, 30 aa, synthetic construct; identical to SEQ ID NO: 29

SEQ ID NO: 42 - protein, 30 aa, synthetic construct; identical to SEQ ID NO: 29

SEQ ID NO: 43 - protein, 30 aa, synthetic construct; identical to SEQ ID NO: 29

SEQ ID NO: 44 - protein, 30 aa, synthetic construct; identical to SEQ ID NO: 29

SEQ ID NO: 45 - protein, 30 aa, synthetic construct; identical to SEQ ID NO: 29

SEQ ID NO: 46 - protein, 30 aa, synthetic construct; identical to SEQ ID NO: 29

SEQ ID NO: 47 - protein, 30 aa, synthetic construct; identical to SEQ ID NO: 29

SEQ ID NO: 48 - protein, 30 aa, synthetic construct; identical to SEQ ID NO: 29

SEQ ID NO: 49 - protein, 30 aa, synthetic construct; identical to SEQ ID NO: 29

SEQ ID NO: 50 - protein, 30 aa, synthetic construct; identical to SEQ ID NO: 29

SEQ ID NO: 51 - protein, 30 aa, synthetic construct; identical to SEQ ID NO: 29

SEQ ID NO: 52 - protein, 30 aa, synthetic construct; identical to SEQ ID NO: 29

SEQ ID NO: 53 - protein, 30 aa, synthetic construct; identical to SEQ ID NO: 29

SEQ ID NO: 54 - protein, 30 aa, synthetic construct; identical to SEQ ID NO: 29

SEQ ID NO: 55 - protein, 30 aa, synthetic construct; identical to SEQ ID NO: 29

SEQ ID NO: 56 - protein, 30 aa, synthetic construct; identical to SEQ ID NO: 29

SEQ ID NO: 57 - protein, 27 aa, synthetic construct
GPOGPOGPOGPOGPOGPOGPOGPOGPO

SEQ ID NO: 58 - protein, 27 aa, synthetic construct
GPOGPOGPOGPOGPOGPOGPOGPOGPO

SEQ ID NO: 59 - protein, 27 aa, synthetic construct
GPOGPOGPOGPOGPOGPOGPOGPOGPO

SEQ ID NO: 60 - protein, 27 aa, synthetic construct
GPOGPOGPOGPOGPOGPOGPOGPOGPO

SEQ ID NO: 61 - protein, 27 aa, synthetic construct
GPOGPOGPOGPOGPOGPOGPOGPOGPO

SEQ ID NO: 62 - protein, 27 aa, synthetic construct
GPOGPOGPOGPOGPOGPOGPOGPOGPO

SEQ ID NO: 63 - protein, 27 aa, synthetic construct
GPOGPOGPOGPOGPOGPOGPOGPOGPO

SEQ ID NO: 64 - protein, 27 aa, synthetic construct
GPOGPOGPOGPOGPOGPOGPOGPOGPO

SEQ ID NO: 65 - protein, 27 aa, synthetic construct
GPOGPOGPOGPOGPOGPOGPOGPOGPO

SEQ ID NO: 66 - protein, 27 aa, synthetic construct
GPOGPOGPOGPOGPOGPOGPOGPOGPO

SEQ ID NO: 67 - protein, 27 aa, synthetic construct
GPOGPOGPOGPOGPOGPOGPOGPOGPO

SEQ ID NO: 68 - protein, 27 aa, synthetic construct
GPOGPOGPOGPOGPOGPOGPOGPOGPO

SEQ ID NO: 69 - protein, 27 aa, synthetic construct
GPOGPOGPOGPOGPOGPOGPOGPOGPO

SEQ ID NO: 70 - protein, 27 aa, synthetic construct
GPOGPOGPOGPOGPOGPOGPOGPOGPO

SEQ ID NO: 71 - protein, 27 aa, synthetic construct
GPOGPOGPOGPOGPOGPOGPOGPOGPO

SEQ ID NO: 72 - protein, 27 aa, synthetic construct
GPOGPOGPOGPOGPOGPOGPOGPOGPO

SEQ ID NO: 73 - protein, 27 aa, synthetic construct
GPOGPOGPOGPOGPOGPOGPOGPOGPO

SEQ ID NO: 74 - protein, 27 aa, synthetic construct
GPOGPOGPOGPOGPOGPOGPOGPOGPO

SEQ ID NO: 75 - protein, 27 aa, synthetic construct
GPOGPOGPOGPOGPOGPOGPOGPOGPO

SEQ ID NO: 76 - protein, 27 aa, synthetic construct
GPOGPOGPOGPOGPOGPOGPOGPOGPO

SEQ ID NO: 77 - protein, 27 aa, synthetic construct
GPOGPOGPOGPOGPOGPOGPOGPOGPO

SEQ ID NO: 78 - protein, 27 aa, synthetic construct
GPOGPOGPOGPOGPOGPOGPOGPOGPO

SEQ ID NO: 79 - protein, 27 aa, synthetic construct
GPOGPOGPOGPOGPOGPOGPOGPOGPO

SEQ ID NO: 80 - protein, 27 aa, synthetic construct
GPOGPOGPOGPOGPOGPOGPOGPOGPO

SEQ ID NO: 81 - protein, 27 aa, synthetic construct
GPOGPOGPOGPOGPOGPOGPOGPOGPO

SEQ ID NO: 82 - protein, 27 aa, synthetic construct
GPOGPOGPOGPOGPOGPOGPOGPOGPO

SEQ ID NO: 83 - protein, 27 aa, synthetic construct
GPOGPOGPOGPOGPOGPOGPOGPOGPO

SEQ ID NO: 84 - protein, 27 aa, synthetic construct
GPOGPOGPOGPOGPOGPOGPOGPOGPO

SEQ ID NO: 85 - protein, 27 aa, synthetic construct
GFOGFOGFOGFOGFOGFOGFOGFOGFO

SEQ ID NO: 86 - protein, 27 aa, synthetic construct
GFOGFOGFOGFOGFOGFOGFOGFOGFO

SEQ ID NO: 87 - protein, 27 aa, synthetic construct
GFOGFOGFOGFOGFOGFOGFOGFOGFO

SEQ ID NO: 88 - protein, 27 aa, synthetic construct
GFOGFOGFOGFOGFOGFOGFOGFOGFO

SEQ ID NO: 89 - protein, 27 aa, synthetic construct
GFOGFOGFOGFOGFOGFOGFOGFOGFO

SEQ ID NO: 90 - protein, 27 aa, synthetic construct
GFOGFOGFOGFOGFOGFOGFOGFOGFO

SEQ ID NO: 91 - protein, 27 aa, synthetic construct
GFOGFOGFOGFOGFOGFOGFOGFOGFO

SEQ ID NO: 92 - protein, 27 aa, synthetic construct
GFOGFOGFOGFOGFOGFOGFOGFOGFO

SEQ ID NO: 93 - protein, 27 aa, synthetic construct
GFOGFOGFOGFOGFOGFOGFOGFOGFO

SEQ ID NO: 94 - protein, 27 aa, synthetic construct
GFOGFOGFOGFOGFOGFOGFOGFOGFO

SEQ ID NO: 95 - protein, 27 aa, synthetic construct
GFOGFOGFOGFOGFOGFOGFOGFOGFO

SEQ ID NO: 96 - protein, 27 aa, synthetic construct
GFOGFOGFOGFOGFOGFOGFOGFOGFO

SEQ ID NO: 97 - protein, 27 aa, synthetic construct
GFOGFOGFOGFOGFOGFOGFOGFOGFO

SEQ ID NO: 98 - protein, 27 aa, synthetic construct
GFOGFOGFOGFOGFOGFOGFOGFOGFO

SEQ ID NO: 99 - protein, 27 aa, synthetic construct
GFOGFOGFOGFOGFOGFOGFOGFOGFO

SEQ ID NO: 100 - protein, 27 aa, synthetic construct
GFOGFOGFOGFOGFOGFOGFOGFOGFO

SEQ ID NO: 101 - protein, 27 aa, synthetic construct
GFOGFOGFOGFOGFOGFOGFOGFOGFO

SEQ ID NO: 102 - protein, 27 aa, synthetic construct
GFOGFOGFOGFOGFOGFOGFOGFOGFO

SEQ ID NO: 103 - protein, 27 aa, synthetic construct
GFOGFOGFOGFOGFOGFOGFOGFOGFO

SEQ ID NO: 104 - protein, 27 aa, synthetic construct
GFOGFOGFOGFOGFOGFOGFOGFOGFO

SEQ ID NO: 105 - protein, 27 aa, synthetic construct
GFOGFOGFOGFOGFOGFOGFOGFOGFO

SEQ ID NO: 106 - protein, 27 aa, synthetic construct
GFOGFOGFOGFOGFOGFOGFOGFOGFO

SEQ ID NO: 107 - protein, 27 aa, synthetic construct
GFOGFOGFOGFOGFOGFOGFOGFOGFO

SEQ ID NO: 108 - protein, 27 aa, synthetic construct
GFOGFOGFOGFOGFOGFOGFOGFOGFO

SEQ ID NO: 109 - protein, 27 aa, synthetic construct
GFOGFOGFOGFOGFOGFOGFOGFOGFO

SEQ ID NO: 110 - protein, 27 aa, synthetic construct
GFOGFOGFOGFOGFOGFOGFOGFOGFO

SEQ ID NO: 111 - protein, 27 aa, synthetic construct
GFOGFOGFOGFOGFOGFOGFOGFOGFO

SEQ ID NO: 112 - protein, 27 aa, synthetic construct
GFOGFOGFOGFOGFOGFOGFOGFOGFO

SEQ ID NO: 113 - protein, 27 aa, synthetic construct
GCOGCOGCOGCOGCOGCOGCOGCOGCO

SEQ ID NO: 114 - protein, 27 aa, synthetic construct
GCOGCOGCOGCOGCOGCOGCOGCOGCO

SEQ ID NO: 115 - protein, 27 aa, synthetic construct
GCOGCOGCOGCOGCOGCOGCOGCOGCO

SEQ ID NO: 116 - protein, 27 aa, synthetic construct
GCOGCOGCOGCOGCOGCOGCOGCOGCO

SEQ ID NO: 117 - protein, 27 aa, synthetic construct
GCOGCOGCOGCOGCOGCOGCOGCOGCO

SEQ ID NO: 118 - protein, 27 aa, synthetic construct
GCOGCOGCOGCOGCOGCOGCOGCOGCO

SEQ ID NO: 119 - protein, 27 aa, synthetic construct
GCOGCOGCOGCOGCOGCOGCOGCOGCO

SEQ ID NO: 120 - protein, 27 aa, synthetic construct
GCOGCOGCOGCOGCOGCOGCOGCOGCO

SEQ ID NO: 121 - protein, 27 aa, synthetic construct
GCOGCOGCOGCOGCOGCOGCOGCOGCO

SEQ ID NO: 122 - protein, 27 aa, synthetic construct
GCOGCOGCOGCOGCOGCOGCOGCOGCO

SEQ ID NO: 123 - protein, 27 aa, synthetic construct
GCOGCOGCOGCOGCOGCOGCOGCOGCO

SEQ ID NO: 124 - protein, 27 aa, synthetic construct
GCOGCOGCOGCOGCOGCOGCOGCOGCO

SEQ ID NO: 125 - protein, 27 aa, synthetic construct
GCOGCOGCOGCOGCOGCOGCOGCOGCO

SEQ ID NO: 126 - protein, 27 aa, synthetic construct
GCOGCOGCOGCOGCOGCOGCOGCOGCO

SEQ ID NO: 127 - protein, 27 aa, synthetic construct
GCOGCOGCOGCOGCOGCOGCOGCOGCO

SEQ ID NO: 128 - protein, 27 aa, synthetic construct
GCOGCOGCOGCOGCOGCOGCOGCOGCO

SEQ ID NO: 129 - protein, 27 aa, synthetic construct
GCOGCOGCOGCOGCOGCOGCOGCOGCO

SEQ ID NO: 130 - protein, 27 aa, synthetic construct
GCOGCOGCOGCOGCOGCOGCOGCOGCO

SEQ ID NO: 131 - protein, 27 aa, synthetic construct
GCOGCOGCOGCOGCOGCOGCOGCOGCO

SEQ ID NO: 132 - protein, 27 aa, synthetic construct
GCOGCOGCOGCOGCOGCOGCOGCOGCO

SEQ ID NO: 133 - protein, 27 aa, synthetic construct
GCOGCOGCOGCOGCOGCOGCOGCOGCO

SEQ ID NO: 134 - protein, 27 aa, synthetic construct
GCOGCOGCOGCOGCOGCOGCOGCOGCO

SEQ ID NO: 135 - protein, 27 aa, synthetic construct
GCOGCOGCOGCOGCOGCOGCOGCOGCO

SEQ ID NO: 136 - protein, 27 aa, synthetic construct
GCOGCOGCOGCOGCOGCOGCOGCOGCO

SEQ ID NO: 137 - protein, 27 aa, synthetic construct
GCOGCOGCOGCOGCOGCOGCOGCOGCO

SEQ ID NO: 138 - protein, 27 aa, synthetic construct
GCOGCOGCOGCOGCOGCOGCOGCOGCO

SEQ ID NO: 139 - protein, 27 aa, synthetic construct
GCOGCOGCOGCOGCOGCOGCOGCOGCO

SEQ ID NO: 140 - protein, 27 aa, synthetic construct
GCOGCOGCOGCOGCOGCOGCOGCOGCO

SEQ ID NO: 141 - protein, 27 aa, synthetic construct
GCOGCOGCOGCOGCOGCOGCOGCOGCO

SEQ ID NO: 142 - protein, 30 aa, synthetic construct
GGGGCOGCOGCOGCOGCOGCOGCOGCOGCO

SEQ ID NO: 143 - protein, 30 aa, synthetic construct; identical to SEQ ID NO: 142

SEQ ID NO: 144 - protein, 30 aa, synthetic construct; identical to SEQ ID NO: 142

SEQ ID NO: 145 - protein, 30 aa, synthetic construct; identical to SEQ ID NO: 142

SEQ ID NO: 146 - protein, 30 aa, synthetic construct; identical to SEQ ID NO: 142

SEQ ID NO: 147 - protein, 30 aa, synthetic construct; identical to SEQ ID NO: 142

SEQ ID NO: 148 - protein, 30 aa, synthetic construct; identical to SEQ ID NO: 142

SEQ ID NO: 149 - protein, 30 aa, synthetic construct; identical to SEQ ID NO: 142

SEQ ID NO: 150 - protein, 30 aa, synthetic construct; identical to SEQ ID NO: 142

SEQ ID NO: 151 - protein, 30 aa, synthetic construct; identical to SEQ ID NO: 142

SEQ ID NO: 152 - protein, 30 aa, synthetic construct; identical to SEQ ID NO: 142

SEQ ID NO: 153 - protein, 30 aa, synthetic construct; identical to SEQ ID NO: 142

SEQ ID NO: 154 - protein, 30 aa, synthetic construct; identical to SEQ ID NO: 142

SEQ ID NO: 155 - protein, 30 aa, synthetic construct; identical to SEQ ID NO: 142

SEQ ID NO: 156 - protein, 30 aa, synthetic construct; identical to SEQ ID NO: 142

SEQ ID NO: 157 - protein, 30 aa, synthetic construct; identical to SEQ ID NO: 142

SEQ ID NO: 158 - protein, 30 aa, synthetic construct; identical to SEQ ID NO: 142

SEQ ID NO: 159 - protein, 30 aa, synthetic construct; identical to SEQ ID NO: 142

SEQ ID NO: 160 - protein, 30 aa, synthetic construct; identical to SEQ ID NO: 142

SEQ ID NO: 161 - protein, 30 aa, synthetic construct; identical to SEQ ID NO: 142

SEQ ID NO: 162 - protein, 30 aa, synthetic construct; identical to SEQ ID NO: 142

SEQ ID NO: 163 - protein, 30 aa, synthetic construct; identical to SEQ ID NO: 142

SEQ ID NO: 164 - protein, 30 aa, synthetic construct; identical to SEQ ID NO: 142

SEQ ID NO: 165 - protein, 30 aa, synthetic construct; identical to SEQ ID NO: 142

SEQ ID NO: 166 - protein, 30 aa, synthetic construct; identical to SEQ ID NO: 142

SEQ ID NO: 167 - protein, 30 aa, synthetic construct; identical to SEQ ID NO: 142

SEQ ID NO: 168 - protein, 30 aa, synthetic construct; identical to SEQ ID NO: 142

SEQ ID NO: 169 - protein, 30 aa, synthetic construct; identical to SEQ ID NO: 142

SEQ ID NO: 170 - protein, 27 aa, synthetic construct
GGGGPOGPOGPOGPLGLAGIGGPOGPO

SEQ ID NO: 171 - protein, 27 aa, synthetic construct
GGGGPOGPOGPOGPLGLAGIGGPOGPO

SEQ ID NO: 172 - protein, 27 aa, synthetic construct
GGGGPOGPOGPOGPLGLAGIGGPOGPO

SEQ ID NO: 173 - protein, 27 aa, synthetic construct
GGGGPOGPOGPOGPLGLAGIGGPOGPO

SEQ ID NO: 174 - protein, 27 aa, synthetic construct
GGGGPOGPOGPOGPLGLAGIGGPOGPO

SEQ ID NO: 175 - protein, 27 aa, synthetic construct
GGGGPOGPOGPOGPLGLAGIGGPOGPO

SEQ ID NO: 176 - protein, 27 aa, synthetic construct
GGGGPOGPOGPOGPLGLAGIGGPOGPO

SEQ ID NO: 177 - protein, 27 aa, synthetic construct
GGGGPOGPOGPOGPLGLAGIGGPOGPO

SEQ ID NO: 178 - protein, 27 aa, synthetic construct
GGGGPOGPOGPOGPLGLAGIGGPOGPO

SEQ ID NO: 179 - protein, 27 aa, synthetic construct
GGGGPOGPOGPOGPLGLAGIGGPOGPO

SEQ ID NO: 180 - protein, 27 aa, synthetic construct
GGGGPOGPOGPOGPLGLAGIGGPOGPO

SEQ ID NO: 181 - protein, 27 aa, synthetic construct
GGGGPOGPOGPOGPLGLAGIGGPOGPO

SEQ ID NO: 182 - protein, 27 aa, synthetic construct
GGGGPOGPOGPOGPLGLAGIGGPOGPO

SEQ ID NO: 183 - protein, 27 aa, synthetic construct
GGGGPOGPOGPOGPLGLAGIGGPOGPO

SEQ ID NO: 184 - protein, 27 aa, synthetic construct
GGGGPOGPOGPOGPLGLAGIGGPOGPO

SEQ ID NO: 185 - protein, 27 aa, synthetic construct
GGGGPOGPOGPOGPLGLAGIGGPOGPO

SEQ ID NO: 186 - protein, 27 aa, synthetic construct
GGGGPOGPOGPOGPLGLAGIGGPOGPO

SEQ ID NO: 187 - protein, 27 aa, synthetic construct
GGGGPOGPOGPOGPLGLAGIGGPOGPO

SEQ ID NO: 188 - protein, 27 aa, synthetic construct
GGGGPOGPOGPOGPLGLAGIGGPOGPO

SEQ ID NO: 189 - protein, 27 aa, synthetic construct
GGGGPOGPOGPOGPLGLAGIGGPOGPO

SEQ ID NO: 190 - protein, 27 aa, synthetic construct
GGGGPOGPOGPOGPLGLAGIGGPOGPO

SEQ ID NO: 191 - protein, 27 aa, synthetic construct
GGGGPOGPOGPOGPLGLAGIGGPOGPO

SEQ ID NO: 192 - protein, 27 aa, synthetic construct
GGGGPOGPOGPOGPLGLAGIGGPOGPO

SEQ ID NO: 193 - protein, 27 aa, synthetic construct
GGGGPOGPOGPOGPLGLAGIGGPOGPO

SEQ ID NO: 194 - protein, 27 aa, synthetic construct
GGGGPOGPOGPOGPLGLAGIGGPOGPO

SEQ ID NO: 195 - protein, 27 aa, synthetic construct
GGGGPOGPOGPOGPLGLAGIGGPOGPO

SEQ ID NO: 196 - protein, 27 aa, synthetic construct
GGGGPOGPOGPOGPLGLAGIGGPOGPO

SEQ ID NO: 197 - protein, 27 aa, synthetic construct
GGGGPOGPOGPOGPLGLAGIGGPOGPO

SEQ ID NO: 198 - protein, 30 aa, synthetic construct
GGGGPOGPOGPOGPLGLAGIGGPOGPOGPO

SEQ ID NO: 199 - protein, 30 aa, synthetic construct; identical to SEQ ID NO: 198

SEQ ID NO: 200 - protein, 30 aa, synthetic construct; identical to SEQ ID NO: 198

SEQ ID NO: 201 - protein, 30 aa, synthetic construct; identical to SEQ ID NO: 198

SEQ ID NO: 202 - protein, 30 aa, synthetic construct; identical to SEQ ID NO: 198

SEQ ID NO: 203 - protein, 30 aa, synthetic construct; identical to SEQ ID NO: 198

SEQ ID NO: 204 - protein, 30 aa, synthetic construct; identical to SEQ ID NO: 198

SEQ ID NO: 205 - protein, 30 aa, synthetic construct; identical to SEQ ID NO: 198

SEQ ID NO: 206 - protein, 30 aa, synthetic construct; identical to SEQ ID NO: 198

SEQ ID NO: 207 - protein, 30 aa, synthetic construct; identical to SEQ ID NO: 198

SEQ ID NO: 208 - protein, 30 aa, synthetic construct; identical to SEQ ID NO: 198

SEQ ID NO: 209 - protein, 30 aa, synthetic construct; identical to SEQ ID NO: 198

SEQ ID NO: 210 - protein, 30 aa, synthetic construct; identical to SEQ ID NO: 198

SEQ ID NO: 211 - protein, 30 aa, synthetic construct; identical to SEQ ID NO: 198

SEQ ID NO: 212 - protein, 30 aa, synthetic construct; identical to SEQ ID NO: 198

SEQ ID NO: 213 - protein, 30 aa, synthetic construct; identical to SEQ ID NO: 198

SEQ ID NO: 214 - protein, 30 aa, synthetic construct; identical to SEQ ID NO: 198

SEQ ID NO: 215 - protein, 30 aa, synthetic construct; identical to SEQ ID NO: 198

SEQ ID NO: 216 - protein, 30 aa, synthetic construct; identical to SEQ ID NO: 198

SEQ ID NO: 217 - protein, 30 aa, synthetic construct; identical to SEQ ID NO: 198

SEQ ID NO: 218 - protein, 30 aa, synthetic construct; identical to SEQ ID NO: 198

SEQ ID NO: 219 - protein, 30 aa, synthetic construct; identical to SEQ ID NO: 198

SEQ ID NO: 220 - protein, 30 aa, synthetic construct; identical to SEQ ID NO: 198

SEQ ID NO: 221 - protein, 30 aa, synthetic construct; identical to SEQ ID NO: 198

SEQ ID NO: 222 - protein, 30 aa, synthetic construct; identical to SEQ ID NO: 198

SEQ ID NO: 223 - protein, 30 aa, synthetic construct; identical to SEQ ID NO: 198

SEQ ID NO: 224 - protein, 30 aa, synthetic construct; identical to SEQ ID NO: 198

SEQ ID NO: 225 - protein, 30 aa, synthetic construct; identical to SEQ ID NO: 198

SEQ ID NO: 226 - protein, 30 aa, synthetic construct
GGGGFOGFOGFOGPLGLAGIGGPOGPOGPO

SEQ ID NO: 227 - protein, 30 aa, synthetic construct; identical to SEQ ID NO: 226

SEQ ID NO: 228 - protein, 30 aa, synthetic construct; identical to SEQ ID NO: 226

SEQ ID NO: 229 - protein, 30 aa, synthetic construct; identical to SEQ ID NO: 226

SEQ ID NO: 230 - protein, 30 aa, synthetic construct; identical to SEQ ID NO: 226

SEQ ID NO: 231 - protein, 30 aa, synthetic construct; identical to SEQ ID NO: 226

SEQ ID NO: 232 - protein, 30 aa, synthetic construct; identical to SEQ ID NO: 226

SEQ ID NO: 233 - protein, 30 aa, synthetic construct; identical to SEQ ID NO: 226

SEQ ID NO: 234 - protein, 30 aa, synthetic construct; identical to SEQ ID NO: 226

SEQ ID NO: 235 - protein, 30 aa, synthetic construct; identical to SEQ ID NO: 226

SEQ ID NO: 236 - protein, 30 aa, synthetic construct; identical to SEQ ID NO: 226

SEQ ID NO: 237 - protein, 30 aa, synthetic construct; identical to SEQ ID NO: 226

SEQ ID NO: 238 - protein, 30 aa, synthetic construct; identical to SEQ ID NO: 226

SEQ ID NO: 239 - protein, 30 aa, synthetic construct; identical to SEQ ID NO: 226

SEQ ID NO: 240 - protein, 30 aa, synthetic construct; identical to SEQ ID NO: 226

SEQ ID NO: 241 - protein, 30 aa, synthetic construct; identical to SEQ ID NO: 226

SEQ ID NO: 242 - protein, 30 aa, synthetic construct; identical to SEQ ID NO: 226

SEQ ID NO: 243 - protein, 30 aa, synthetic construct; identical to SEQ ID NO: 226

SEQ ID NO: 244 - protein, 30 aa, synthetic construct; identical to SEQ ID NO: 226

SEQ ID NO: 245 - protein, 30 aa, synthetic construct; identical to SEQ ID NO: 226

SEQ ID NO: 246 - protein, 30 aa, synthetic construct; identical to SEQ ID NO: 226

SEQ ID NO: 247 - protein, 30 aa, synthetic construct; identical to SEQ ID NO: 226

SEQ ID NO: 248 - protein, 30 aa, synthetic construct; identical to SEQ ID NO: 226

SEQ ID NO: 249 - protein, 30 aa, synthetic construct; identical to SEQ ID NO: 226

SEQ ID NO: 250 - protein, 30 aa, synthetic construct; identical to SEQ ID NO: 226

SEQ ID NO: 251 - protein, 30 aa, synthetic construct; identical to SEQ ID NO: 226

SEQ ID NO: 252 - protein, 30 aa, synthetic construct; identical to SEQ ID NO: 226

SEQ ID NO: 253 - protein, 30 aa, synthetic construct; identical to SEQ ID NO: 226

SEQ ID NO: 254 - protein, 27 aa, synthetic construct
GGGGFOGFOGFOGPLGLAGIGGPOGPO

SEQ ID NO: 255 - protein, 27 aa, synthetic construct
GGGGFOGFOGFOGPLGLAGIGGPOGPO

SEQ ID NO: 256 - protein, 27 aa, synthetic construct
GGGGFOGFOGFOGPLGLAGIGGPOGPO

SEQ ID NO: 257 - protein, 27 aa, synthetic construct
GGGGFOGFOGFOGPLGLAGIGGPOGPO

SEQ ID NO: 258 - protein, 27 aa, synthetic construct
GGGGFOGFOGFOGPLGLAGIGGPOGPO

SEQ ID NO: 259 - protein, 27 aa, synthetic construct
GGGGFOGFOGFOGPLGLAGIGGPOGPO

SEQ ID NO: 260 - protein, 27 aa, synthetic construct
GGGGFOGFOGFOGPLGLAGIGGPOGPO

SEQ ID NO: 261 - protein, 27 aa, synthetic construct
GGGGFOGFOGFOGPLGLAGIGGPOGPO

SEQ ID NO: 262 - protein, 27 aa, synthetic construct
GGGGFOGFOGFOGPLGLAGIGGPOGPO

SEQ ID NO: 263 - protein, 27 aa, synthetic construct
GGGGFOGFOGFOGPLGLAGIGGPOGPO

SEQ ID NO: 264 - protein, 27 aa, synthetic construct
GGGGFOGFOGFOGPLGLAGIGGPOGPO

SEQ ID NO: 265 - protein, 27 aa, synthetic construct
GGGGFOGFOGFOGPLGLAGIGGPOGPO

SEQ ID NO: 266 - protein, 27 aa, synthetic construct
GGGGFOGFOGFOGPLGLAGIGGPOGPO

SEQ ID NO: 267 - protein, 27 aa, synthetic construct
GGGGFOGFOGFOGPLGLAGIGGPOGPO

SEQ ID NO: 268 - protein, 27 aa, synthetic construct
GGGGFOGFOGFOGPLGLAGIGGPOGPO

SEQ ID NO: 269 - protein, 27 aa, synthetic construct
GGGGFOGFOGFOGPLGLAGIGGPOGPO

SEQ ID NO: 270 - protein, 27 aa, synthetic construct
GGGGFOGFOGFOGPLGLAGIGGPOGPO

SEQ ID NO: 271 - protein, 27 aa, synthetic construct
GGGGFOGFOGFOGPLGLAGIGGPOGPO

SEQ ID NO: 272 - protein, 27 aa, synthetic construct
GGGGFOGFOGFOGPLGLAGIGGPOGPO

SEQ ID NO: 273 - protein, 27 aa, synthetic construct
GGGGFOGFOGFOGPLGLAGIGGPOGPO

SEQ ID NO: 274 - protein, 27 aa, synthetic construct
GGGGFOGFOGFOGPLGLAGIGGPOGPO

SEQ ID NO: 275 - protein, 27 aa, synthetic construct
GGGGFOGFOGFOGPLGLAGIGGPOGPO

SEQ ID NO: 276 - protein, 27 aa, synthetic construct
GGGGFOGFOGFOGPLGLAGIGGPOGPO

SEQ ID NO: 277 - protein, 27 aa, synthetic construct
GGGGFOGFOGFOGPLGLAGIGGPOGPO

SEQ ID NO: 278 - protein, 27 aa, synthetic construct
GGGGFOGFOGFOGPLGLAGIGGPOGPO

SEQ ID NO: 279 - protein, 27 aa, synthetic construct
GGGGFOGFOGFOGPLGLAGIGGPOGPO

SEQ ID NO: 280 - protein, 27 aa, synthetic construct
GGGGFOGFOGFOGPLGLAGIGGPOGPO

SEQ ID NO: 281 - protein, 27 aa, synthetic construct
GGGGFOGFOGFOGPLGLAGIGGPOGPO

SEQ ID NO: 282 - protein, 27 aa, synthetic construct
GGGGFOGFOGFOGPLGLAGIGGFOGFO

SEQ ID NO: 283 - protein, 27 aa, synthetic construct
GGGGFOGFOGFOGPLGLAGIGGFOGFO

SEQ ID NO: 284 - protein, 27 aa, synthetic construct
GGGGFOGFOGFOGPLGLAGIGGFOGFO

SEQ ID NO: 285 - protein, 27 aa, synthetic construct
GGGGFOGFOGFOGPLGLAGIGGFOGFO

SEQ ID NO: 286 - protein, 27 aa, synthetic construct
GGGGFOGFOGFOGPLGLAGIGGFOGFO

SEQ ID NO: 287 - protein, 27 aa, synthetic construct
GGGGFOGFOGFOGPLGLAGIGGFOGFO

SEQ ID NO: 288 - protein, 27 aa, synthetic construct
GGGGFOGFOGFOGPLGLAGIGGFOGFO

SEQ ID NO: 289 - protein, 27 aa, synthetic construct
GGGGFOGFOGFOGPLGLAGIGGFOGFO

SEQ ID NO: 290 - protein, 27 aa, synthetic construct
GGGGFOGFOGFOGPLGLAGIGGFOGFO

SEQ ID NO: 291 - protein, 27 aa, synthetic construct
GGGGFOGFOGFOGPLGLAGIGGFOGFO

SEQ ID NO: 292 - protein, 27 aa, synthetic construct
GGGGFOGFOGFOGPLGLAGIGGFOGFO

SEQ ID NO: 293 - protein, 27 aa, synthetic construct
GGGGFOGFOGFOGPLGLAGIGGFOGFO

SEQ ID NO: 294 - protein, 27 aa, synthetic construct
GGGGFOGFOGFOGPLGLAGIGGFOGFO

SEQ ID NO: 295 - protein, 27 aa, synthetic construct
GGGGFOGFOGFOGPLGLAGIGGFOGFO

SEQ ID NO: 296 - protein, 27 aa, synthetic construct
GGGGFOGFOGFOGPLGLAGIGGFOGFO

SEQ ID NO: 297 - protein, 27 aa, synthetic construct
GGGGFOGFOGFOGPLGLAGIGGFOGFO

SEQ ID NO: 298 - protein, 27 aa, synthetic construct
GGGGFOGFOGFOGPLGLAGIGGFOGFO

SEQ ID NO: 299 - protein, 27 aa, synthetic construct
GGGGFOGFOGFOGPLGLAGIGGFOGFO

SEQ ID NO: 300 - protein, 27 aa, synthetic construct
GGGGFOGFOGFOGPLGLAGIGGFOGFO

SEQ ID NO: 301 - protein, 27 aa, synthetic construct
GGGGFOGFOGFOGPLGLAGIGGFOGFO

SEQ ID NO: 302 - protein, 27 aa, synthetic construct
GGGGFOGFOGFOGPLGLAGIGGFOGFO

SEQ ID NO: 303 - protein, 27 aa, synthetic construct
GGGGFOGFOGFOGPLGLAGIGGFOGFO

SEQ ID NO: 304 - protein, 27 aa, synthetic construct
GGGGFOGFOGFOGPLGLAGIGGFOGFO

SEQ ID NO: 305 - protein, 27 aa, synthetic construct
GGGGFOGFOGFOGPLGLAGIGGFOGFO

SEQ ID NO: 306 - protein, 27 aa, synthetic construct
GGGGFOGFOGFOGPLGLAGIGGFOGFO

SEQ ID NO: 307 - protein, 27 aa, synthetic construct
GGGGFOGFOGFOGPLGLAGIGGFOGFO

SEQ ID NO: 308 - protein, 27 aa, synthetic construct
GGGGFOGFOGFOGPLGLAGIGGFOGFO

SEQ ID NO: 309 - protein, 27 aa, synthetic construct
GGGGFOGFOGFOGPLGLAGIGGFOGFO

SEQ ID NO: 310 - protein, 30 aa, synthetic construct
GGGGFOGFOGFOGPLGLAGIGGFOGFOGFO

SEQ ID NO: 311 - protein, 30 aa, synthetic construct; identical to SEQ ID NO: 310

SEQ ID NO: 312 - protein, 30 aa, synthetic construct; identical to SEQ ID NO: 310

SEQ ID NO: 313 - protein, 30 aa, synthetic construct; identical to SEQ ID NO: 310

SEQ ID NO: 314 - protein, 30 aa, synthetic construct; identical to SEQ ID NO: 310

SEQ ID NO: 315 - protein, 30 aa, synthetic construct; identical to SEQ ID NO: 310

SEQ ID NO: 316 - protein, 30 aa, synthetic construct; identical to SEQ ID NO: 310

SEQ ID NO: 317 - protein, 30 aa, synthetic construct; identical to SEQ ID NO: 310

SEQ ID NO: 318 - protein, 30 aa, synthetic construct; identical to SEQ ID NO: 310

SEQ ID NO: 319 - protein, 30 aa, synthetic construct; identical to SEQ ID NO: 310

SEQ ID NO: 320 - protein, 30 aa, synthetic construct; identical to SEQ ID NO: 310

SEQ ID NO: 321 - protein, 30 aa, synthetic construct; identical to SEQ ID NO: 310

SEQ ID NO: 322 - protein, 30 aa, synthetic construct; identical to SEQ ID NO: 310

SEQ ID NO: 323 - protein, 30 aa, synthetic construct; identical to SEQ ID NO: 310

SEQ ID NO: 324 - protein, 30 aa, synthetic construct; identical to SEQ ID NO: 310

SEQ ID NO: 325 - protein, 30 aa, synthetic construct; identical to SEQ ID NO: 310

SEQ ID NO: 326 - protein, 30 aa, synthetic construct; identical to SEQ ID NO: 310

SEQ ID NO: 327 - protein, 30 aa, synthetic construct; identical to SEQ ID NO: 310

SEQ ID NO: 328 - protein, 30 aa, synthetic construct; identical to SEQ ID NO: 310

SEQ ID NO: 329 - protein, 30 aa, synthetic construct; identical to SEQ ID NO: 310

SEQ ID NO: 330 - protein, 30 aa, synthetic construct; identical to SEQ ID NO: 310

SEQ ID NO: 331 - protein, 30 aa, synthetic construct; identical to SEQ ID NO: 310

SEQ ID NO: 332 - protein, 30 aa, synthetic construct; identical to SEQ ID NO: 310

SEQ ID NO: 333 - protein, 30 aa, synthetic construct; identical to SEQ ID NO: 310

SEQ ID NO: 334 - protein, 30 aa, synthetic construct; identical to SEQ ID NO: 310

SEQ ID NO: 335 - protein, 30 aa, synthetic construct; identical to SEQ ID NO: 310

SEQ ID NO: 336 - protein, 30 aa, synthetic construct; identical to SEQ ID NO: 310

SEQ ID NO: 337 - protein, 30 aa, synthetic construct; identical to SEQ ID NO: 310

SEQ ID NO: 338 - protein, 27 aa, synthetic construct
GGGGPOGPOGPOGPQGIAGQGGPOGPO

SEQ ID NO: 339 - protein, 27 aa, synthetic construct
GGGGPOGPOGPOGPQGIAGQGGPOGPO

SEQ ID NO: 340 - protein, 27 aa, synthetic construct
GGGGPOGPOGPOGPQGIAGQGGPOGPO

SEQ ID NO: 341 - protein, 27 aa, synthetic construct
GGGGPOGPOGPOGPQGIAGQGGPOGPO

SEQ ID NO: 342 - protein, 27 aa, synthetic construct
GGGGPOGPOGPOGPQGIAGQGGPOGPO

SEQ ID NO: 343 - protein, 27 aa, synthetic construct
GGGGPOGPOGPOGPQGIAGQGGPOGPO

SEQ ID NO: 344 - protein, 27 aa, synthetic construct
GGGGPOGPOGPOGPQGIAGQGGPOGPO

SEQ ID NO: 345 - protein, 27 aa, synthetic construct
GGGGPOGPOGPOGPQGIAGQGGPOGPO

SEQ ID NO: 346 - protein, 27 aa, synthetic construct
GGGGPOGPOGPOGPQGIAGQGGPOGPO

SEQ ID NO: 347 - protein, 27 aa, synthetic construct
GGGGPOGPOGPOGPQGIAGQGGPOGPO

SEQ ID NO: 348 - protein, 27 aa, synthetic construct
GGGGPOGPOGPOGPQGIAGQGGPOGPO

SEQ ID NO: 349 - protein, 27 aa, synthetic construct
GGGGPOGPOGPOGPQGIAGQGGPOGPO

SEQ ID NO: 350 - protein, 27 aa, synthetic construct
GGGGPOGPOGPOGPQGIAGQGGPOGPO

SEQ ID NO: 351 - protein, 27 aa, synthetic construct
GGGGPOGPOGPOGPQGIAGQGGPOGPO

SEQ ID NO: 352 - protein, 27 aa, synthetic construct
GGGGPOGPOGPOGPQGIAGQGGPOGPO

SEQ ID NO: 353 - protein, 27 aa, synthetic construct
GGGGPOGPOGPOGPQGIAGQGGPOGPO

SEQ ID NO: 354 - protein, 27 aa, synthetic construct
GGGGPOGPOGPOGPQGIAGQGGPOGPO

SEQ ID NO: 355 - protein, 27 aa, synthetic construct
GGGGPOGPOGPOGPQGIAGQGGPOGPO

SEQ ID NO: 356 - protein, 27 aa, synthetic construct
GGGGPOGPOGPOGPQGIAGQGGPOGPO

SEQ ID NO: 357 - protein, 27 aa, synthetic construct
GGGGPOGPOGPOGPQGIAGQGGPOGPO

SEQ ID NO: 358 - protein, 27 aa, synthetic construct
GGGGPOGPOGPOGPQGIAGQGGPOGPO

SEQ ID NO: 359 - protein, 27 aa, synthetic construct
GGGGPOGPOGPOGPQGIAGQGGPOGPO

SEQ ID NO: 360 - protein, 27 aa, synthetic construct
GGGGPOGPOGPOGPQGIAGQGGPOGPO

SEQ ID NO: 361 - protein, 27 aa, synthetic construct
GGGGPOGPOGPOGPQGIAGQGGPOGPO

SEQ ID NO: 362 - protein, 27 aa, synthetic construct
GGGGPOGPOGPOGPQGIAGQGGPOGPO

SEQ ID NO: 363 - protein, 27 aa, synthetic construct
GGGGPOGPOGPOGPQGIAGQGGPOGPO

SEQ ID NO: 364 - protein, 27 aa, synthetic construct
GGGGPOGPOGPOGPQGIAGQGGPOGPO

SEQ ID NO: 365 - protein, 27 aa, synthetic construct
GGGGPOGPOGPOGPQGIAGQGGPOGPO

SEQ ID NO: 366 - protein, 30 aa, synthetic construct
GGGGPOGPOGPOGPQGIAGQGGPOGPOGPO

SEQ ID NO: 367 - protein, 30 aa, synthetic construct; identical to SEQ ID NO: 366

SEQ ID NO: 368 - protein, 30 aa, synthetic construct; identical to SEQ ID NO: 366

SEQ ID NO: 369 - protein, 30 aa, synthetic construct; identical to SEQ ID NO: 366

SEQ ID NO: 370 - protein, 30 aa, synthetic construct; identical to SEQ ID NO: 366

SEQ ID NO: 371 - protein, 30 aa, synthetic construct; identical to SEQ ID NO: 366

SEQ ID NO: 372 - protein, 30 aa, synthetic construct; identical to SEQ ID NO: 366

SEQ ID NO: 373 - protein, 30 aa, synthetic construct; identical to SEQ ID NO: 366

SEQ ID NO: 374 - protein, 30 aa, synthetic construct; identical to SEQ ID NO: 366

SEQ ID NO: 375 - protein, 30 aa, synthetic construct; identical to SEQ ID NO: 366

SEQ ID NO: 376 - protein, 30 aa, synthetic construct; identical to SEQ ID NO: 366

SEQ ID NO: 377 - protein, 30 aa, synthetic construct; identical to SEQ ID NO: 366

SEQ ID NO: 378 - protein, 30 aa, synthetic construct; identical to SEQ ID NO: 366

SEQ ID NO: 379 - protein, 30 aa, synthetic construct; identical to SEQ ID NO: 366

SEQ ID NO: 380 - protein, 30 aa, synthetic construct; identical to SEQ ID NO: 366

SEQ ID NO: 381 - protein, 30 aa, synthetic construct; identical to SEQ ID NO: 366

SEQ ID NO: 382 - protein, 30 aa, synthetic construct; identical to SEQ ID NO: 366

SEQ ID NO: 383 - protein, 30 aa, synthetic construct; identical to SEQ ID NO: 366

SEQ ID NO: 384 - protein, 30 aa, synthetic construct; identical to SEQ ID NO: 366

SEQ ID NO: 385 - protein, 30 aa, synthetic construct; identical to SEQ ID NO: 366

SEQ ID NO: 386 - protein, 30 aa, synthetic construct; identical to SEQ ID NO: 366

SEQ ID NO: 387 - protein, 30 aa, synthetic construct; identical to SEQ ID NO: 366

SEQ ID NO: 388 - protein, 30 aa, synthetic construct; identical to SEQ ID NO: 366

SEQ ID NO: 389 - protein, 30 aa, synthetic construct; identical to SEQ ID NO: 366

SEQ ID NO: 390 - protein, 30 aa, synthetic construct; identical to SEQ ID NO: 366

SEQ ID NO: 391 - protein, 30 aa, synthetic construct; identical to SEQ ID NO: 366

SEQ ID NO: 392 - protein, 30 aa, synthetic construct; identical to SEQ ID NO: 366

SEQ ID NO: 393 - protein, 30 aa, synthetic construct; identical to SEQ ID NO: 366

SEQ ID NO: 394 - protein, 30 aa, synthetic construct
GGGGFOGFOGFOGPQGIAGQGGPOGPOGPO

SEQ ID NO: 395 - protein, 30 aa, synthetic construct; identical to SEQ ID NO: 394

SEQ ID NO: 396 - protein, 30 aa, synthetic construct; identical to SEQ ID NO: 394

SEQ ID NO: 397 - protein, 30 aa, synthetic construct; identical to SEQ ID NO: 394

SEQ ID NO: 398 - protein, 30 aa, synthetic construct; identical to SEQ ID NO: 394

SEQ ID NO: 399 - protein, 30 aa, synthetic construct; identical to SEQ ID NO: 394

SEQ ID NO: 400 - protein, 30 aa, synthetic construct; identical to SEQ ID NO: 394

SEQ ID NO: 401 - protein, 30 aa, synthetic construct; identical to SEQ ID NO: 394

SEQ ID NO: 402 - protein, 30 aa, synthetic construct; identical to SEQ ID NO: 394

SEQ ID NO: 403 - protein, 30 aa, synthetic construct; identical to SEQ ID NO: 394

SEQ ID NO: 404 - protein, 30 aa, synthetic construct; identical to SEQ ID NO: 394

SEQ ID NO: 405 - protein, 30 aa, synthetic construct; identical to SEQ ID NO: 394

SEQ ID NO: 406 - protein, 30 aa, synthetic construct; identical to SEQ ID NO: 394

SEQ ID NO: 407 - protein, 30 aa, synthetic construct; identical to SEQ ID NO: 394

SEQ ID NO: 408 - protein, 30 aa, synthetic construct; identical to SEQ ID NO: 394

SEQ ID NO: 409 - protein, 30 aa, synthetic construct; identical to SEQ ID NO: 394

SEQ ID NO: 410 - protein, 30 aa, synthetic construct; identical to SEQ ID NO: 394

SEQ ID NO: 411 - protein, 30 aa, synthetic construct; identical to SEQ ID NO: 394

SEQ ID NO: 412 - protein, 30 aa, synthetic construct; identical to SEQ ID NO: 394

SEQ ID NO: 413 - protein, 30 aa, synthetic construct; identical to SEQ ID NO: 394

SEQ ID NO: 414 - protein, 30 aa, synthetic construct; identical to SEQ ID NO: 394

SEQ ID NO: 415 - protein, 30 aa, synthetic construct; identical to SEQ ID NO: 394

SEQ ID NO: 416 - protein, 30 aa, synthetic construct; identical to SEQ ID NO: 394

SEQ ID NO: 417 - protein, 30 aa, synthetic construct; identical to SEQ ID NO: 394

SEQ ID NO: 418 - protein, 30 aa, synthetic construct; identical to SEQ ID NO: 394

SEQ ID NO: 419 - protein, 30 aa, synthetic construct; identical to SEQ ID NO: 394

SEQ ID NO: 420 - protein, 30 aa, synthetic construct; identical to SEQ ID NO: 394

SEQ ID NO: 421 - protein, 30 aa, synthetic construct; identical to SEQ ID NO: 394

SEQ ID NO: 422 - protein, 27 aa, synthetic construct
GGGGFOGFOGFOGPQGIAGQGGPOGPO

SEQ ID NO: 423 - protein, 27 aa, synthetic construct
GGGGFOGFOGFOGPQGIAGQGGPOGPO

SEQ ID NO: 424 - protein, 27 aa, synthetic construct
GGGGFOGFOGFOGPQGIAGQGGPOGPO

SEQ ID NO: 425 - protein, 27 aa, synthetic construct
GGGGFOGFOGFOGPQGIAGQGGPOGPO

SEQ ID NO: 426 - protein, 27 aa, synthetic construct
GGGGFOGFOGFOGPQGIAGQGGPOGPO

SEQ ID NO: 427 - protein, 27 aa, synthetic construct
GGGGFOGFOGFOGPQGIAGQGGPOGPO

SEQ ID NO: 428 - protein, 27 aa, synthetic construct
GGGGFOGFOGFOGPQGIAGQGGPOGPO

SEQ ID NO: 429 - protein, 27 aa, synthetic construct
GGGGFOGFOGFOGPQGIAGQGGPOGPO

SEQ ID NO: 430 - protein, 27 aa, synthetic construct
GGGGFOGFOGFOGPQGIAGQGGPOGPO

SEQ ID NO: 431 - protein, 27 aa, synthetic construct
GGGGFOGFOGFOGPQGIAGQGGPOGPO

SEQ ID NO: 432 - protein, 27 aa, synthetic construct
GGGGFOGFOGFOGPQGIAGQGGPOGPO

SEQ ID NO: 433 - protein, 27 aa, synthetic construct
GGGGFOGFOGFOGPQGIAGQGGPOGPO

SEQ ID NO: 434 - protein, 27 aa, synthetic construct
GGGGFOGFOGFOGPQGIAGQGGPOGPO

SEQ ID NO: 435 - protein, 27 aa, synthetic construct
GGGGFOGFOGFOGPQGIAGQGGPOGPO

SEQ ID NO: 436 - protein, 27 aa, synthetic construct
GGGGFOGFOGFOGPQGIAGQGGPOGPO

SEQ ID NO: 437 - protein, 27 aa, synthetic construct
GGGGFOGFOGFOGPQGIAGQGGPOGPO

SEQ ID NO: 438 - protein, 27 aa, synthetic construct
GGGGFOGFOGFOGPQGIAGQGGPOGPO

SEQ ID NO: 439 - protein, 27 aa, synthetic construct
GGGGFOGFOGFOGPQGIAGQGGPOGPO

SEQ ID NO: 440 - protein, 27 aa, synthetic construct
GGGGFOGFOGFOGPQGIAGQGGPOGPO

SEQ ID NO: 441 - protein, 27 aa, synthetic construct
GGGGFOGFOGFOGPQGIAGQGGPOGPO

SEQ ID NO: 442 - protein, 27 aa, synthetic construct
GGGGFOGFOGFOGPQGIAGQGGPOGPO

SEQ ID NO: 443 - protein, 27 aa, synthetic construct
GGGGFOGFOGFOGPQGIAGQGGPOGPO

SEQ ID NO: 444 - protein, 27 aa, synthetic construct
GGGGFOGFOGFOGPQGIAGQGGPOGPO

SEQ ID NO: 445 - protein, 27 aa, synthetic construct
GGGGFOGFOGFOGPQGIAGQGGPOGPO

SEQ ID NO: 446 - protein, 27 aa, synthetic construct
GGGGFOGFOGFOGPQGIAGQGGPOGPO

SEQ ID NO: 447 - protein, 27 aa, synthetic construct
GGGGFOGFOGFOGPQGIAGQGGPOGPO

SEQ ID NO: 448 - protein, 27 aa, synthetic construct
GGGGFOGFOGFOGPQGIAGQGGPOGPO

SEQ ID NO: 449 - protein, 27 aa, synthetic construct
GGGGFOGFOGFOGPQGIAGQGGPOGPO

SEQ ID NO: 450 - protein, 27 aa, synthetic construct
GGGGFOGFOGFOGPQGIAGQGGFOGFO

SEQ ID NO: 451 - protein, 27 aa, synthetic construct
GGGGFOGFOGFOGPQGIAGQGGFOGFO

SEQ ID NO: 452 - protein, 27 aa, synthetic construct
GGGGFOGFOGFOGPQGIAGQGGFOGFO

SEQ ID NO: 453 - protein, 27 aa, synthetic construct
GGGGFOGFOGFOGPQGIAGQGGFOGFO

SEQ ID NO: 454 - protein, 27 aa, synthetic construct
GGGGFOGFOGFOGPQGIAGQGGFOGFO

SEQ ID NO: 455 - protein, 27 aa, synthetic construct
GGGGFOGFOGFOGPQGIAGQGGFOGFO

SEQ ID NO: 456 - protein, 27 aa, synthetic construct
GGGGFOGFOGFOGPQGIAGQGGFOGFO

SEQ ID NO: 457 - protein, 27 aa, synthetic construct
GGGGFOGFOGFOGPQGIAGQGGFOGFO

SEQ ID NO: 458 - protein, 27 aa, synthetic construct
GGGGFOGFOGFOGPQGIAGQGGFOGFO

SEQ ID NO: 459 - protein, 27 aa, synthetic construct
GGGGFOGFOGFOGPQGIAGQGGFOGFO

SEQ ID NO: 460 - protein, 27 aa, synthetic construct
GGGGFOGFOGFOGPQGIAGQGGFOGFO

SEQ ID NO: 461 - protein, 27 aa, synthetic construct
GGGGFOGFOGFOGPQGIAGQGGFOGFO

SEQ ID NO: 462 - protein, 27 aa, synthetic construct
GGGGFOGFOGFOGPQGIAGQGGFOGFO

SEQ ID NO: 463 - protein, 27 aa, synthetic construct
GGGGFOGFOGFOGPQGIAGQGGFOGFO

SEQ ID NO: 464 - protein, 27 aa, synthetic construct
GGGGFOGFOGFOGPQGIAGQGGFOGFO

SEQ ID NO: 465 - protein, 27 aa, synthetic construct
GGGGFOGFOGFOGPQGIAGQGGFOGFO

SEQ ID NO: 466 - protein, 27 aa, synthetic construct
GGGGFOGFOGFOGPQGIAGQGGFOGFO

SEQ ID NO: 467 - protein, 27 aa, synthetic construct
GGGGFOGFOGFOGPQGIAGQGGFOGFO

SEQ ID NO: 468 - protein, 27 aa, synthetic construct
GGGGFOGFOGFOGPQGIAGQGGFOGFO

SEQ ID NO: 469 - protein, 27 aa, synthetic construct
GGGGFOGFOGFOGPQGIAGQGGFOGFO

SEQ ID NO: 470 - protein, 27 aa, synthetic construct
GGGGFOGFOGFOGPQGIAGQGGFOGFO

SEQ ID NO: 471 - protein, 27 aa, synthetic construct
GGGGFOGFOGFOGPQGIAGQGGFOGFO

SEQ ID NO: 472 - protein, 27 aa, synthetic construct
GGGGFOGFOGFOGPQGIAGQGGFOGFO

SEQ ID NO: 473 - protein, 27 aa, synthetic construct
GGGGFOGFOGFOGPQGIAGQGGFOGFO

SEQ ID NO: 474 - protein, 27 aa, synthetic construct
GGGGFOGFOGFOGPQGIAGQGGFOGFO

SEQ ID NO: 475 - protein, 27 aa, synthetic construct
GGGGFOGFOGFOGPQGIAGQGGFOGFO

SEQ ID NO: 476 - protein, 27 aa, synthetic construct
GGGGFOGFOGFOGPQGIAGQGGFOGFO

SEQ ID NO: 477 - protein, 27 aa, synthetic construct
GGGGFOGFOGFOGPQGIAGQGGFOGFO

SEQ ID NO: 478 - protein, 30 aa, synthetic construct
GGGGFOGFOGFOGPQGIAGQGGFOGFOGFO

SEQ ID NO: 479 - protein, 30 aa, synthetic construct; identical to SEQ ID NO: 478

SEQ ID NO: 480 - protein, 30 aa, synthetic construct; identical to SEQ ID NO: 478

SEQ ID NO: 481 - protein, 30 aa, synthetic construct; identical to SEQ ID NO: 478

SEQ ID NO: 482 - protein, 30 aa, synthetic construct; identical to SEQ ID NO: 478

SEQ ID NO: 483 - protein, 30 aa, synthetic construct; identical to SEQ ID NO: 478

SEQ ID NO: 484 - protein, 30 aa, synthetic construct; identical to SEQ ID NO: 478

SEQ ID NO: 485 - protein, 30 aa, synthetic construct; identical to SEQ ID NO: 478

SEQ ID NO: 486 - protein, 30 aa, synthetic construct; identical to SEQ ID NO: 478

SEQ ID NO: 487 - protein, 30 aa, synthetic construct; identical to SEQ ID NO: 478

SEQ ID NO: 488 - protein, 30 aa, synthetic construct; identical to SEQ ID NO: 478

SEQ ID NO: 489 - protein, 30 aa, synthetic construct; identical to SEQ ID NO: 478

SEQ ID NO: 490 - protein, 30 aa, synthetic construct; identical to SEQ ID NO: 478

SEQ ID NO: 491 - protein, 30 aa, synthetic construct; identical to SEQ ID NO: 478

SEQ ID NO: 492 - protein, 30 aa, synthetic construct; identical to SEQ ID NO: 478

SEQ ID NO: 493 - protein, 30 aa, synthetic construct; identical to SEQ ID NO: 478

SEQ ID NO: 494 - protein, 30 aa, synthetic construct; identical to SEQ ID NO: 478

SEQ ID NO: 495 - protein, 30 aa, synthetic construct; identical to SEQ ID NO: 478

SEQ ID NO: 496 - protein, 30 aa, synthetic construct; identical to SEQ ID NO: 478

SEQ ID NO: 497 - protein, 30 aa, synthetic construct; identical to SEQ ID NO: 478

SEQ ID NO: 498 - protein, 30 aa, synthetic construct; identical to SEQ ID NO: 478

SEQ ID NO: 499 - protein, 30 aa, synthetic construct; identical to SEQ ID NO: 478

SEQ ID NO: 500 - protein, 30 aa, synthetic construct; identical to SEQ ID NO: 478

SEQ ID NO: 501 - protein, 30 aa, synthetic construct; identical to SEQ ID NO: 478

SEQ ID NO: 502 - protein, 30 aa, synthetic construct; identical to SEQ ID NO: 478

SEQ ID NO: 503 - protein, 30 aa, synthetic construct; identical to SEQ ID NO: 478

SEQ ID NO: 504 - protein, 30 aa, synthetic construct; identical to SEQ ID NO: 478

SEQ ID NO: 505 - protein, 30 aa, synthetic construct; identical to SEQ ID NO: 478

SEQ ID NO: 506 - protein, 27 aa, synthetic construct
GGGGPOGPOGPOGPLGIAGIGGPOGPO

SEQ ID NO: 507 - protein, 27 aa, synthetic construct
GGGGPOGPOGPOGPLGIAGIGGPOGPO

SEQ ID NO: 508 - protein, 27 aa, synthetic construct
GGGGPOGPOGPOGPLGIAGIGGPOGPO

SEQ ID NO: 509 - protein, 27 aa, synthetic construct
GGGGPOGPOGPOGPLGIAGIGGPOGPO

SEQ ID NO: 510 - protein, 27 aa, synthetic construct
GGGGPOGPOGPOGPLGIAGIGGPOGPO

SEQ ID NO: 511 - protein, 27 aa, synthetic construct
GGGGPOGPOGPOGPLGIAGIGGPOGPO

SEQ ID NO: 512 - protein, 27 aa, synthetic construct
GGGGPOGPOGPOGPLGIAGIGGPOGPO

SEQ ID NO: 513 - protein, 27 aa, synthetic construct
GGGGPOGPOGPOGPLGIAGIGGPOGPO

SEQ ID NO: 514 - protein, 27 aa, synthetic construct
GGGGPOGPOGPOGPLGIAGIGGPOGPO

SEQ ID NO: 515 - protein, 27 aa, synthetic construct
GGGGPOGPOGPOGPLGIAGIGGPOGPO

SEQ ID NO: 516 - protein, 27 aa, synthetic construct
GGGGPOGPOGPOGPLGIAGIGGPOGPO

SEQ ID NO: 517 - protein, 27 aa, synthetic construct
GGGGPOGPOGPOGPLGIAGIGGPOGPO

SEQ ID NO: 518 - protein, 27 aa, synthetic construct
GGGGPOGPOGPOGPLGIAGIGGPOGPO

SEQ ID NO: 519 - protein, 27 aa, synthetic construct
GGGGPOGPOGPOGPLGIAGIGGPOGPO

SEQ ID NO: 520 - protein, 27 aa, synthetic construct
GGGGPOGPOGPOGPLGIAGIGGPOGPO

SEQ ID NO: 521 - protein, 27 aa, synthetic construct
GGGGPOGPOGPOGPLGIAGIGGPOGPO

SEQ ID NO: 522 - protein, 27 aa, synthetic construct
GGGGPOGPOGPOGPLGIAGIGGPOGPO

SEQ ID NO: 523 - protein, 27 aa, synthetic construct
GGGGPOGPOGPOGPLGIAGIGGPOGPO

SEQ ID NO: 524 - protein, 27 aa, synthetic construct
GGGGPOGPOGPOGPLGIAGIGGPOGPO

SEQ ID NO: 525 - protein, 27 aa, synthetic construct
GGGGPOGPOGPOGPLGIAGIGGPOGPO

SEQ ID NO: 526 - protein, 27 aa, synthetic construct
GGGGPOGPOGPOGPLGIAGIGGPOGPO

SEQ ID NO: 527 - protein, 27 aa, synthetic construct
GGGGPOGPOGPOGPLGIAGIGGPOGPO

SEQ ID NO: 528 - protein, 27 aa, synthetic construct
GGGGPOGPOGPOGPLGIAGIGGPOGPO

SEQ ID NO: 529 - protein, 27 aa, synthetic construct
GGGGPOGPOGPOGPLGIAGIGGPOGPO

SEQ ID NO: 530 - protein, 27 aa, synthetic construct
GGGGPOGPOGPOGPLGIAGIGGPOGPO

SEQ ID NO: 531 - protein, 27 aa, synthetic construct
GGGGPOGPOGPOGPLGIAGIGGPOGPO

SEQ ID NO: 532 - protein, 27 aa, synthetic construct
GGGGPOGPOGPOGPLGIAGIGGPOGPO

SEQ ID NO: 533 - protein, 27 aa, synthetic construct
GGGGPOGPOGPOGPLGIAGIGGPOGPO

SEQ ID NO: 534 - protein, 30 aa, synthetic construct
GGGGPOGPOGPOGPLGIAGIGGPOGPOGPO

SEQ ID NO: 535 - protein, 30 aa, synthetic construct; identical to SEQ ID NO: 534

SEQ ID NO: 536 - protein, 30 aa, synthetic construct; identical to SEQ ID NO: 534

SEQ ID NO: 537 - protein, 30 aa, synthetic construct; identical to SEQ ID NO: 534

SEQ ID NO: 538 - protein, 30 aa, synthetic construct; identical to SEQ ID NO: 534

SEQ ID NO: 539 - protein, 30 aa, synthetic construct; identical to SEQ ID NO: 534

SEQ ID NO: 540 - protein, 30 aa, synthetic construct; identical to SEQ ID NO: 534

SEQ ID NO: 541 - protein, 30 aa, synthetic construct; identical to SEQ ID NO: 534

SEQ ID NO: 542 - protein, 30 aa, synthetic construct; identical to SEQ ID NO: 534

SEQ ID NO: 543 - protein, 30 aa, synthetic construct; identical to SEQ ID NO: 534

SEQ ID NO: 544 - protein, 30 aa, synthetic construct; identical to SEQ ID NO: 534

SEQ ID NO: 545 - protein, 30 aa, synthetic construct; identical to SEQ ID NO: 534

SEQ ID NO: 546 - protein, 30 aa, synthetic construct; identical to SEQ ID NO: 534

SEQ ID NO: 547 - protein, 30 aa, synthetic construct; identical to SEQ ID NO: 534

SEQ ID NO: 548 - protein, 30 aa, synthetic construct; identical to SEQ ID NO: 534

SEQ ID NO: 549 - protein, 30 aa, synthetic construct; identical to SEQ ID NO: 534

SEQ ID NO: 550 - protein, 30 aa, synthetic construct; identical to SEQ ID NO: 534

SEQ ID NO: 551 - protein, 30 aa, synthetic construct; identical to SEQ ID NO: 534

SEQ ID NO: 552 - protein, 30 aa, synthetic construct; identical to SEQ ID NO: 534

SEQ ID NO: 553 - protein, 30 aa, synthetic construct; identical to SEQ ID NO: 534

SEQ ID NO: 554 - protein, 30 aa, synthetic construct; identical to SEQ ID NO: 534

SEQ ID NO: 555 - protein, 30 aa, synthetic construct; identical to SEQ ID NO: 534

SEQ ID NO: 556 - protein, 30 aa, synthetic construct; identical to SEQ ID NO: 534

SEQ ID NO: 557 - protein, 30 aa, synthetic construct; identical to SEQ ID NO: 534

SEQ ID NO: 558 - protein, 30 aa, synthetic construct; identical to SEQ ID NO: 534

SEQ ID NO: 559 - protein, 30 aa, synthetic construct; identical to SEQ ID NO: 534

SEQ ID NO: 560 - protein, 30 aa, synthetic construct; identical to SEQ ID NO: 534

SEQ ID NO: 561 - protein, 30 aa, synthetic construct; identical to SEQ ID NO: 534

SEQ ID NO: 562 - protein, 30 aa, synthetic construct
GGGGFOGFOGFOGPLGIAGIGGPOGPOGPO

SEQ ID NO: 563 - protein, 30 aa, synthetic construct; identical to SEQ ID NO: 562

SEQ ID NO: 564 - protein, 30 aa, synthetic construct; identical to SEQ ID NO: 562

SEQ ID NO: 565 - protein, 30 aa, synthetic construct; identical to SEQ ID NO: 562

SEQ ID NO: 566 - protein, 30 aa, synthetic construct; identical to SEQ ID NO: 562

SEQ ID NO: 567 - protein, 30 aa, synthetic construct; identical to SEQ ID NO: 562

SEQ ID NO: 568 - protein, 30 aa, synthetic construct; identical to SEQ ID NO: 562

SEQ ID NO: 569 - protein, 30 aa, synthetic construct; identical to SEQ ID NO: 562

SEQ ID NO: 570 - protein, 30 aa, synthetic construct; identical to SEQ ID NO: 562

SEQ ID NO: 571 - protein, 30 aa, synthetic construct; identical to SEQ ID NO: 562

SEQ ID NO: 572 - protein, 30 aa, synthetic construct; identical to SEQ ID NO: 562

SEQ ID NO: 573 - protein, 30 aa, synthetic construct; identical to SEQ ID NO: 562

SEQ ID NO: 574 - protein, 30 aa, synthetic construct; identical to SEQ ID NO: 562

SEQ ID NO: 575 - protein, 30 aa, synthetic construct; identical to SEQ ID NO: 562

SEQ ID NO: 576 - protein, 30 aa, synthetic construct; identical to SEQ ID NO: 562

SEQ ID NO: 577 - protein, 30 aa, synthetic construct; identical to SEQ ID NO: 562

SEQ ID NO: 578 - protein, 30 aa, synthetic construct; identical to SEQ ID NO: 562

SEQ ID NO: 579 - protein, 30 aa, synthetic construct; identical to SEQ ID NO: 562

SEQ ID NO: 580 - protein, 30 aa, synthetic construct; identical to SEQ ID NO: 562

SEQ ID NO: 581 - protein, 30 aa, synthetic construct; identical to SEQ ID NO: 562

SEQ ID NO: 582 - protein, 30 aa, synthetic construct; identical to SEQ ID NO: 562

SEQ ID NO: 583 - protein, 30 aa, synthetic construct; identical to SEQ ID NO: 562

SEQ ID NO: 584 - protein, 30 aa, synthetic construct; identical to SEQ ID NO: 562

SEQ ID NO: 585 - protein, 30 aa, synthetic construct; identical to SEQ ID NO: 562

SEQ ID NO: 586 - protein, 30 aa, synthetic construct; identical to SEQ ID NO: 562

SEQ ID NO: 587 - protein, 30 aa, synthetic construct; identical to SEQ ID NO: 562

SEQ ID NO: 588 - protein, 30 aa, synthetic construct; identical to SEQ ID NO: 562

SEQ ID NO: 589 - protein, 30 aa, synthetic construct; identical to SEQ ID NO: 562

SEQ ID NO: 590 - protein, 27 aa, synthetic construct
GGGGFOGFOGFOGPLGIAGIGGPOGPO

SEQ ID NO: 591 - protein, 27 aa, synthetic construct
GGGGFOGFOGFOGPLGIAGIGGPOGPO

SEQ ID NO: 592 - protein, 27 aa, synthetic construct
GGGGFOGFOGFOGPLGIAGIGGPOGPO

SEQ ID NO: 593 - protein, 27 aa, synthetic construct
GGGGFOGFOGFOGPLGIAGIGGPOGPO

SEQ ID NO: 594 - protein, 27 aa, synthetic construct
GGGGFOGFOGFOGPLGIAGIGGPOGPO

SEQ ID NO: 595 - protein, 27 aa, synthetic construct
GGGGFOGFOGFOGPLGIAGIGGPOGPO

SEQ ID NO: 596 - protein, 27 aa, synthetic construct
GGGGFOGFOGFOGPLGIAGIGGPOGPO

SEQ ID NO: 597 - protein, 27 aa, synthetic construct
GGGGFOGFOGFOGPLGIAGIGGPOGPO

SEQ ID NO: 598 - protein, 27 aa, synthetic construct
GGGGFOGFOGFOGPLGIAGIGGPOGPO

SEQ ID NO: 599 - protein, 27 aa, synthetic construct
GGGGFOGFOGFOGPLGIAGIGGPOGPO

SEQ ID NO: 600 - protein, 27 aa, synthetic construct
GGGGFOGFOGFOGPLGIAGIGGPOGPO

SEQ ID NO: 601 - protein, 27 aa, synthetic construct
GGGGFOGFOGFOGPLGIAGIGGPOGPO

SEQ ID NO: 602 - protein, 27 aa, synthetic construct
GGGGFOGFOGFOGPLGIAGIGGPOGPO

SEQ ID NO: 603 - protein, 27 aa, synthetic construct
GGGGFOGFOGFOGPLGIAGIGGPOGPO

SEQ ID NO: 604 - protein, 27 aa, synthetic construct
GGGGFOGFOGFOGPLGIAGIGGPOGPO

SEQ ID NO: 605 - protein, 27 aa, synthetic construct
GGGGFOGFOGFOGPLGIAGIGGPOGPO

SEQ ID NO: 606 - protein, 27 aa, synthetic construct
GGGGFOGFOGFOGPLGIAGIGGPOGPO

SEQ ID NO: 607 - protein, 27 aa, synthetic construct
GGGGFOGFOGFOGPLGIAGIGGPOGPO

SEQ ID NO: 608 - protein, 27 aa, synthetic construct
GGGGFOGFOGFOGPLGIAGIGGPOGPO

SEQ ID NO: 609 - protein, 27 aa, synthetic construct
GGGGFOGFOGFOGPLGIAGIGGPOGPO

SEQ ID NO: 610 - protein, 27 aa, synthetic construct
GGGGFOGFOGFOGPLGIAGIGGPOGPO

SEQ ID NO: 611 - protein, 27 aa, synthetic construct
GGGGFOGFOGFOGPLGIAGIGGPOGPO

SEQ ID NO: 612 - protein, 27 aa, synthetic construct
GGGGFOGFOGFOGPLGIAGIGGPOGPO

SEQ ID NO: 613 - protein, 27 aa, synthetic construct
GGGGFOGFOGFOGPLGIAGIGGPOGPO

SEQ ID NO: 614 - protein, 27 aa, synthetic construct
GGGGFOGFOGFOGPLGIAGIGGPOGPO

SEQ ID NO: 615 - protein, 27 aa, synthetic construct
GGGGFOGFOGFOGPLGIAGIGGPOGPO

SEQ ID NO: 616 - protein, 27 aa, synthetic construct
GGGGFOGFOGFOGPLGIAGIGGPOGPO

SEQ ID NO: 617 - protein, 27 aa, synthetic construct
GGGGFOGFOGFOGPLGIAGIGGPOGPO

SEQ ID NO: 618 - protein, 27 aa, synthetic construct
GGGGFOGFOGFOGPLGIAGIGGFOGFO

SEQ ID NO: 619 - protein, 27 aa, synthetic construct
GGGGFOGFOGFOGPLGIAGIGGFOGFO

SEQ ID NO: 620 - protein, 27 aa, synthetic construct
GGGGFOGFOGFOGPLGIAGIGGFOGFO

SEQ ID NO: 621 - protein, 27 aa, synthetic construct
GGGGFOGFOGFOGPLGIAGIGGFOGFO

SEQ ID NO: 622 - protein, 27 aa, synthetic construct
GGGGFOGFOGFOGPLGIAGIGGFOGFO

SEQ ID NO: 623 - protein, 27 aa, synthetic construct
GGGGFOGFOGFOGPLGIAGIGGFOGFO

SEQ ID NO: 624 - protein, 27 aa, synthetic construct
GGGGFOGFOGFOGPLGIAGIGGFOGFO

SEQ ID NO: 625 - protein, 27 aa, synthetic construct
GGGGFOGFOGFOGPLGIAGIGGFOGFO

SEQ ID NO: 626 - protein, 27 aa, synthetic construct
GGGGFOGFOGFOGPLGIAGIGGFOGFO

SEQ ID NO: 627 - protein, 27 aa, synthetic construct
GGGGFOGFOGFOGPLGIAGIGGFOGFO

SEQ ID NO: 628 - protein, 27 aa, synthetic construct
GGGGFOGFOGFOGPLGIAGIGGFOGFO

SEQ ID NO: 629 - protein, 27 aa, synthetic construct
GGGGFOGFOGFOGPLGIAGIGGFOGFO

SEQ ID NO: 630 - protein, 27 aa, synthetic construct
GGGGFOGFOGFOGPLGIAGIGGFOGFO

SEQ ID NO: 631 - protein, 27 aa, synthetic construct
GGGGFOGFOGFOGPLGIAGIGGFOGFO

SEQ ID NO: 632 - protein, 27 aa, synthetic construct
GGGGFOGFOGFOGPLGIAGIGGFOGFO

SEQ ID NO: 633 - protein, 27 aa, synthetic construct
GGGGFOGFOGFOGPLGIAGIGGFOGFO

SEQ ID NO: 634 - protein, 27 aa, synthetic construct
GGGGFOGFOGFOGPLGIAGIGGFOGFO

SEQ ID NO: 635 - protein, 27 aa, synthetic construct
GGGGFOGFOGFOGPLGIAGIGGFOGFO

SEQ ID NO: 636 - protein, 27 aa, synthetic construct
GGGGFOGFOGFOGPLGIAGIGGFOGFO

SEQ ID NO: 637 - protein, 27 aa, synthetic construct
GGGGFOGFOGFOGPLGIAGIGGFOGFO

SEQ ID NO: 638 - protein, 27 aa, synthetic construct
GGGGFOGFOGFOGPLGIAGIGGFOGFO

SEQ ID NO: 639 - protein, 27 aa, synthetic construct
GGGGFOGFOGFOGPLGIAGIGGFOGFO

SEQ ID NO: 640 - protein, 27 aa, synthetic construct
GGGGFOGFOGFOGPLGIAGIGGFOGFO

SEQ ID NO: 641 - protein, 27 aa, synthetic construct
GGGGFOGFOGFOGPLGIAGIGGFOGFO

SEQ ID NO: 642 - protein, 27 aa, synthetic construct
GGGGFOGFOGFOGPLGIAGIGGFOGFO

SEQ ID NO: 643 - protein, 27 aa, synthetic construct
GGGGFOGFOGFOGPLGIAGIGGFOGFO

SEQ ID NO: 644 - protein, 27 aa, synthetic construct
GGGGFOGFOGFOGPLGIAGIGGFOGFO

SEQ ID NO: 645 - protein, 27 aa, synthetic construct
GGGGFOGFOGFOGPLGIAGIGGFOGFO

SEQ ID NO: 646 - protein, 30 aa, synthetic construct
GGGGFOGFOGFOGPLGIAGIGGFOGFOGFO

SEQ ID NO: 647 - protein, 30 aa, synthetic construct; identical to SEQ ID NO: 646

SEQ ID NO: 648 - protein, 30 aa, synthetic construct; identical to SEQ ID NO: 646

SEQ ID NO: 649 - protein, 30 aa, synthetic construct; identical to SEQ ID NO: 646

SEQ ID NO: 650 - protein, 30 aa, synthetic construct; identical to SEQ ID NO: 646

SEQ ID NO: 651 - protein, 30 aa, synthetic construct; identical to SEQ ID NO: 646

SEQ ID NO: 652 - protein, 30 aa, synthetic construct; identical to SEQ ID NO: 646

SEQ ID NO: 653 - protein, 30 aa, synthetic construct; identical to SEQ ID NO: 646

SEQ ID NO: 654 - protein, 30 aa, synthetic construct; identical to SEQ ID NO: 646

SEQ ID NO: 655 - protein, 30 aa, synthetic construct; identical to SEQ ID NO: 646

SEQ ID NO: 656 - protein, 30 aa, synthetic construct; identical to SEQ ID NO: 646

SEQ ID NO: 657 - protein, 30 aa, synthetic construct; identical to SEQ ID NO: 646

SEQ ID NO: 658 - protein, 30 aa, synthetic construct; identical to SEQ ID NO: 646

SEQ ID NO: 659 - protein, 30 aa, synthetic construct; identical to SEQ ID NO: 646

SEQ ID NO: 660 - protein, 30 aa, synthetic construct; identical to SEQ ID NO: 646

SEQ ID NO: 661 - protein, 30 aa, synthetic construct; identical to SEQ ID NO: 646

SEQ ID NO: 662 - protein, 30 aa, synthetic construct; identical to SEQ ID NO: 646

SEQ ID NO: 663 - protein, 30 aa, synthetic construct; identical to SEQ ID NO: 646

SEQ ID NO: 664 - protein, 30 aa, synthetic construct; identical to SEQ ID NO: 646

SEQ ID NO: 665 - protein, 30 aa, synthetic construct; identical to SEQ ID NO: 646

SEQ ID NO: 666 - protein, 30 aa, synthetic construct; identical to SEQ ID NO: 646

SEQ ID NO: 667 - protein, 30 aa, synthetic construct; identical to SEQ ID NO: 646

SEQ ID NO: 668 - protein, 30 aa, synthetic construct; identical to SEQ ID NO: 646

SEQ ID NO: 669 - protein, 30 aa, synthetic construct; identical to SEQ ID NO: 646

SEQ ID NO: 670 - protein, 30 aa, synthetic construct; identical to SEQ ID NO: 646

SEQ ID NO: 671 - protein, 30 aa, synthetic construct; identical to SEQ ID NO: 646

SEQ ID NO: 672 - protein, 30 aa, synthetic construct; identical to SEQ ID NO: 646

SEQ ID NO: 673 - protein, 30 aa, synthetic construct; identical to SEQ ID NO: 646

SEQ ID NO: 674 - protein, 33 aa, synthetic construct
GGGGPOGPOGPOGPLGIAGITGARGLAGPOGPO

SEQ ID NO: 675 - protein, 33 aa, synthetic construct; identical to SEQ ID NO: 674

SEQ ID NO: 676 - protein, 33 aa, synthetic construct; identical to SEQ ID NO: 674

SEQ ID NO: 677 - protein, 33 aa, synthetic construct; identical to SEQ ID NO: 674

SEQ ID NO: 678 - protein, 33 aa, synthetic construct; identical to SEQ ID NO: 674

SEQ ID NO: 679 - protein, 33 aa, synthetic construct; identical to SEQ ID NO: 674

SEQ ID NO: 680 - protein, 33 aa, synthetic construct; identical to SEQ ID NO: 674

SEQ ID NO: 681 - protein, 33 aa, synthetic construct; identical to SEQ ID NO: 674

SEQ ID NO: 682 - protein, 33 aa, synthetic construct; identical to SEQ ID NO: 674

SEQ ID NO: 683 - protein, 33 aa, synthetic construct; identical to SEQ ID NO: 674

SEQ ID NO: 684 - protein, 33 aa, synthetic construct; identical to SEQ ID NO: 674

SEQ ID NO: 685 - protein, 33 aa, synthetic construct; identical to SEQ ID NO: 674

SEQ ID NO: 686 - protein, 33 aa, synthetic construct; identical to SEQ ID NO: 674

SEQ ID NO: 687 - protein, 33 aa, synthetic construct; identical to SEQ ID NO: 674

SEQ ID NO: 688 - protein, 33 aa, synthetic construct; identical to SEQ ID NO: 674

SEQ ID NO: 689 - protein, 33 aa, synthetic construct; identical to SEQ ID NO: 674

SEQ ID NO: 690 - protein, 33 aa, synthetic construct; identical to SEQ ID NO: 674

SEQ ID NO: 691 - protein, 33 aa, synthetic construct; identical to SEQ ID NO: 674

SEQ ID NO: 692 - protein, 33 aa, synthetic construct; identical to SEQ ID NO: 674

SEQ ID NO: 693 - protein, 33 aa, synthetic construct; identical to SEQ ID NO: 674

SEQ ID NO: 694 - protein, 33 aa, synthetic construct; identical to SEQ ID NO: 674

SEQ ID NO: 695 - protein, 33 aa, synthetic construct; identical to SEQ ID NO: 674

SEQ ID NO: 696 - protein, 33 aa, synthetic construct; identical to SEQ ID NO: 674

SEQ ID NO: 697 - protein, 33 aa, synthetic construct; identical to SEQ ID NO: 674

SEQ ID NO: 698 - protein, 33 aa, synthetic construct; identical to SEQ ID NO: 674

SEQ ID NO: 699 - protein, 33 aa, synthetic construct; identical to SEQ ID NO: 674

SEQ ID NO: 700 - protein, 33 aa, synthetic construct; identical to SEQ ID NO: 674

SEQ ID NO: 701 - protein, 33 aa, synthetic construct; identical to SEQ ID NO: 674

SEQ ID NO: 702 - protein, 36 aa, synthetic construct
GGGGPOGPOGPOGPLGIAGITGARGLAGPOGPOGPO

SEQ ID NO: 703 - protein, 36 aa, synthetic construct; identical to SEQ ID NO: 702

SEQ ID NO: 704 - protein, 36 aa, synthetic construct; identical to SEQ ID NO: 702

SEQ ID NO: 705 - protein, 36 aa, synthetic construct; identical to SEQ ID NO: 702

SEQ ID NO: 706 - protein, 36 aa, synthetic construct; identical to SEQ ID NO: 702

SEQ ID NO: 707 - protein, 36 aa, synthetic construct; identical to SEQ ID NO: 702

SEQ ID NO: 708 - protein, 36 aa, synthetic construct; identical to SEQ ID NO: 702

SEQ ID NO: 709 - protein, 36 aa, synthetic construct; identical to SEQ ID NO: 702

SEQ ID NO: 710 - protein, 36 aa, synthetic construct; identical to SEQ ID NO: 702

SEQ ID NO: 711 - protein, 36 aa, synthetic construct; identical to SEQ ID NO: 702

SEQ ID NO: 712 - protein, 36 aa, synthetic construct; identical to SEQ ID NO: 702

SEQ ID NO: 713 - protein, 36 aa, synthetic construct; identical to SEQ ID NO: 702

SEQ ID NO: 714 - protein, 36 aa, synthetic construct; identical to SEQ ID NO: 702

SEQ ID NO: 715 - protein, 36 aa, synthetic construct; identical to SEQ ID NO: 702

SEQ ID NO: 716 - protein, 36 aa, synthetic construct; identical to SEQ ID NO: 702

SEQ ID NO: 717 - protein, 36 aa, synthetic construct; identical to SEQ ID NO: 702

SEQ ID NO: 718 - protein, 36 aa, synthetic construct; identical to SEQ ID NO: 702

SEQ ID NO: 719 - protein, 36 aa, synthetic construct; identical to SEQ ID NO: 702

SEQ ID NO: 720 - protein, 36 aa, synthetic construct; identical to SEQ ID NO: 702

SEQ ID NO: 721 - protein, 36 aa, synthetic construct; identical to SEQ ID NO: 702

SEQ ID NO: 722 - protein, 36 aa, synthetic construct; identical to SEQ ID NO: 702

SEQ ID NO: 723 - protein, 36 aa, synthetic construct; identical to SEQ ID NO: 702

SEQ ID NO: 724 - protein, 36 aa, synthetic construct; identical to SEQ ID NO: 702

SEQ ID NO: 725 - protein, 36 aa, synthetic construct; identical to SEQ ID NO: 702

SEQ ID NO: 726 - protein, 36 aa, synthetic construct; identical to SEQ ID NO: 702

SEQ ID NO: 727 - protein, 36 aa, synthetic construct; identical to SEQ ID NO: 702

SEQ ID NO: 728 - protein, 36 aa, synthetic construct; identical to SEQ ID NO: 702

SEQ ID NO: 729 - protein, 36 aa, synthetic construct; identical to SEQ ID NO: 702

SEQ ID NO: 730 - protein, 36 aa, synthetic construct
GGGGFOGFOGFOGPLGIAGITGARGLAGPOGPOGPO

SEQ ID NO: 731 - protein, 36 aa, synthetic construct; identical to SEQ ID NO: 730

SEQ ID NO: 732 - protein, 36 aa, synthetic construct; identical to SEQ ID NO: 730

SEQ ID NO: 733 - protein, 36 aa, synthetic construct; identical to SEQ ID NO: 730

SEQ ID NO: 734 - protein, 36 aa, synthetic construct; identical to SEQ ID NO: 730

SEQ ID NO: 735 - protein, 36 aa, synthetic construct; identical to SEQ ID NO: 730

SEQ ID NO: 736 - protein, 36 aa, synthetic construct; identical to SEQ ID NO: 730

SEQ ID NO: 737 - protein, 36 aa, synthetic construct; identical to SEQ ID NO: 730

SEQ ID NO: 738 - protein, 36 aa, synthetic construct; identical to SEQ ID NO: 730

SEQ ID NO: 739 - protein, 36 aa, synthetic construct; identical to SEQ ID NO: 730

SEQ ID NO: 740 - protein, 36 aa, synthetic construct; identical to SEQ ID NO: 730

SEQ ID NO: 741 - protein, 36 aa, synthetic construct; identical to SEQ ID NO: 730

SEQ ID NO: 742 - protein, 36 aa, synthetic construct; identical to SEQ ID NO: 730

SEQ ID NO: 743 - protein, 36 aa, synthetic construct; identical to SEQ ID NO: 730

SEQ ID NO: 744 - protein, 36 aa, synthetic construct; identical to SEQ ID NO: 730

SEQ ID NO: 745 - protein, 36 aa, synthetic construct; identical to SEQ ID NO: 730

SEQ ID NO: 746 - protein, 36 aa, synthetic construct; identical to SEQ ID NO: 730

SEQ ID NO: 747 - protein, 36 aa, synthetic construct; identical to SEQ ID NO: 730

SEQ ID NO: 748 - protein, 36 aa, synthetic construct; identical to SEQ ID NO: 730

SEQ ID NO: 749 - protein, 36 aa, synthetic construct; identical to SEQ ID NO: 730

SEQ ID NO: 750 - protein, 36 aa, synthetic construct; identical to SEQ ID NO: 730

SEQ ID NO: 751 - protein, 36 aa, synthetic construct; identical to SEQ ID NO: 730

SEQ ID NO: 752 - protein, 36 aa, synthetic construct; identical to SEQ ID NO: 730

SEQ ID NO: 753 - protein, 36 aa, synthetic construct; identical to SEQ ID NO: 730

SEQ ID NO: 754 - protein, 36 aa, synthetic construct; identical to SEQ ID NO: 730

SEQ ID NO: 755 - protein, 36 aa, synthetic construct; identical to SEQ ID NO: 730

SEQ ID NO: 756 - protein, 36 aa, synthetic construct; identical to SEQ ID NO: 730

SEQ ID NO: 757 - protein, 36 aa, synthetic construct; identical to SEQ ID NO: 730

SEQ ID NO: 758 - protein, 33 aa, synthetic construct
GGGGFOGFOGFOGPLGIAGITGARGLAGPOGPO

SEQ ID NO: 759 - protein, 33 aa, synthetic construct; identical to SEQ ID NO: 758

SEQ ID NO: 760 - protein, 33 aa, synthetic construct; identical to SEQ ID NO: 758

SEQ ID NO: 761 - protein, 33 aa, synthetic construct; identical to SEQ ID NO: 758

SEQ ID NO: 762 - protein, 33 aa, synthetic construct; identical to SEQ ID NO: 758

SEQ ID NO: 763 - protein, 33 aa, synthetic construct; identical to SEQ ID NO: 758

SEQ ID NO: 764 - protein, 33 aa, synthetic construct; identical to SEQ ID NO: 758

SEQ ID NO: 765 - protein, 33 aa, synthetic construct; identical to SEQ ID NO: 758

SEQ ID NO: 766 - protein, 33 aa, synthetic construct; identical to SEQ ID NO: 758

SEQ ID NO: 767 - protein, 33 aa, synthetic construct; identical to SEQ ID NO: 758

SEQ ID NO: 768 - protein, 33 aa, synthetic construct; identical to SEQ ID NO: 758

SEQ ID NO: 769 - protein, 33 aa, synthetic construct; identical to SEQ ID NO: 758

SEQ ID NO: 770 - protein, 33 aa, synthetic construct; identical to SEQ ID NO: 758

SEQ ID NO: 771 - protein, 33 aa, synthetic construct; identical to SEQ ID NO: 758

SEQ ID NO: 772 - protein, 33 aa, synthetic construct; identical to SEQ ID NO: 758

SEQ ID NO: 773 - protein, 33 aa, synthetic construct; identical to SEQ ID NO: 758

SEQ ID NO: 774 - protein, 33 aa, synthetic construct; identical to SEQ ID NO: 758

SEQ ID NO: 775 - protein, 33 aa, synthetic construct; identical to SEQ ID NO: 758

SEQ ID NO: 776 - protein, 33 aa, synthetic construct; identical to SEQ ID NO: 758

SEQ ID NO: 777 - protein, 33 aa, synthetic construct; identical to SEQ ID NO: 758

SEQ ID NO: 778 - protein, 33 aa, synthetic construct; identical to SEQ ID NO: 758

SEQ ID NO: 779 - protein, 33 aa, synthetic construct; identical to SEQ ID NO: 758

SEQ ID NO: 780 - protein, 33 aa, synthetic construct; identical to SEQ ID NO: 758

SEQ ID NO: 781 - protein, 33 aa, synthetic construct; identical to SEQ ID NO: 758

SEQ ID NO: 782 - protein, 33 aa, synthetic construct; identical to SEQ ID NO: 758

SEQ ID NO: 783 - protein, 33 aa, synthetic construct; identical to SEQ ID NO: 758

SEQ ID NO: 784 - protein, 33 aa, synthetic construct; identical to SEQ ID NO: 758

SEQ ID NO: 785 - protein, 33 aa, synthetic construct; identical to SEQ ID NO: 758

SEQ ID NO: 786 - protein, 33 aa, synthetic construct
GGGGFOGFOGFOGPLGIAGITGARGLAGFOGFO

SEQ ID NO: 787 - protein, 33 aa, synthetic construct; identical to SEQ ID NO: 786

SEQ ID NO: 788 - protein, 33 aa, synthetic construct; identical to SEQ ID NO: 786

SEQ ID NO: 789 - protein, 33 aa, synthetic construct; identical to SEQ ID NO: 786

SEQ ID NO: 790 - protein, 33 aa, synthetic construct; identical to SEQ ID NO: 786

SEQ ID NO: 791 - protein, 33 aa, synthetic construct; identical to SEQ ID NO: 786

SEQ ID NO: 792 - protein, 33 aa, synthetic construct; identical to SEQ ID NO: 786

SEQ ID NO: 793 - protein, 33 aa, synthetic construct; identical to SEQ ID NO: 786

SEQ ID NO: 794 - protein, 33 aa, synthetic construct; identical to SEQ ID NO: 786

SEQ ID NO: 795 - protein, 33 aa, synthetic construct; identical to SEQ ID NO: 786

SEQ ID NO: 796 - protein, 33 aa, synthetic construct; identical to SEQ ID NO: 786

SEQ ID NO: 797 - protein, 33 aa, synthetic construct; identical to SEQ ID NO: 786

SEQ ID NO: 798 - protein, 33 aa, synthetic construct; identical to SEQ ID NO: 786

SEQ ID NO: 799 - protein, 33 aa, synthetic construct; identical to SEQ ID NO: 786

SEQ ID NO: 800 - protein, 33 aa, synthetic construct; identical to SEQ ID NO: 786

SEQ ID NO: 801 - protein, 33 aa, synthetic construct; identical to SEQ ID NO: 786

SEQ ID NO: 802 - protein, 33 aa, synthetic construct; identical to SEQ ID NO: 786

SEQ ID NO: 803 - protein, 33 aa, synthetic construct; identical to SEQ ID NO: 786

SEQ ID NO: 804 - protein, 33 aa, synthetic construct; identical to SEQ ID NO: 786

SEQ ID NO: 805 - protein, 33 aa, synthetic construct; identical to SEQ ID NO: 786

SEQ ID NO: 806 - protein, 33 aa, synthetic construct; identical to SEQ ID NO: 786

SEQ ID NO: 807 - protein, 33 aa, synthetic construct; identical to SEQ ID NO: 786

SEQ ID NO: 808 - protein, 33 aa, synthetic construct; identical to SEQ ID NO: 786

SEQ ID NO: 809 - protein, 33 aa, synthetic construct; identical to SEQ ID NO: 786

SEQ ID NO: 810 - protein, 33 aa, synthetic construct; identical to SEQ ID NO: 786

SEQ ID NO: 811 - protein, 33 aa, synthetic construct; identical to SEQ ID NO: 786

SEQ ID NO: 812 - protein, 33 aa, synthetic construct; identical to SEQ ID NO: 786

SEQ ID NO: 813 - protein, 33 aa, synthetic construct; identical to SEQ ID NO: 786

SEQ ID NO: 814 - protein, 36 aa, synthetic construct
GGGGFOGFOGFOGPLGIAGITGARGLAGFOGFOGFO

SEQ ID NO: 815 - protein, 36 aa, synthetic construct; identical to SEQ ID NO: 814

SEQ ID NO: 816 - protein, 36 aa, synthetic construct; identical to SEQ ID NO: 814

SEQ ID NO: 817 - protein, 36 aa, synthetic construct; identical to SEQ ID NO: 814

SEQ ID NO: 818 - protein, 36 aa, synthetic construct; identical to SEQ ID NO: 814

SEQ ID NO: 819 - protein, 36 aa, synthetic construct; identical to SEQ ID NO: 814

SEQ ID NO: 820 - protein, 36 aa, synthetic construct; identical to SEQ ID NO: 814

SEQ ID NO: 821 - protein, 36 aa, synthetic construct; identical to SEQ ID NO: 814

SEQ ID NO: 822 - protein, 36 aa, synthetic construct; identical to SEQ ID NO: 814

SEQ ID NO: 823 - protein, 36 aa, synthetic construct; identical to SEQ ID NO: 814

SEQ ID NO: 824 - protein, 36 aa, synthetic construct; identical to SEQ ID NO: 814

SEQ ID NO: 825 - protein, 36 aa, synthetic construct; identical to SEQ ID NO: 814

SEQ ID NO: 826 - protein, 36 aa, synthetic construct; identical to SEQ ID NO: 814

SEQ ID NO: 827 - protein, 36 aa, synthetic construct; identical to SEQ ID NO: 814

SEQ ID NO: 828 - protein, 36 aa, synthetic construct; identical to SEQ ID NO: 814

SEQ ID NO: 829 - protein, 36 aa, synthetic construct; identical to SEQ ID NO: 814

SEQ ID NO: 830 - protein, 36 aa, synthetic construct; identical to SEQ ID NO: 814

SEQ ID NO: 831 - protein, 36 aa, synthetic construct; identical to SEQ ID NO: 814

SEQ ID NO: 832 - protein, 36 aa, synthetic construct; identical to SEQ ID NO: 814

SEQ ID NO: 833 - protein, 36 aa, synthetic construct; identical to SEQ ID NO: 814

SEQ ID NO: 834 - protein, 36 aa, synthetic construct; identical to SEQ ID NO: 814

SEQ ID NO: 835 - protein, 36 aa, synthetic construct; identical to SEQ ID NO: 814

SEQ ID NO: 836 - protein, 36 aa, synthetic construct; identical to SEQ ID NO: 814

SEQ ID NO: 837 - protein, 36 aa, synthetic construct; identical to SEQ ID NO: 814

SEQ ID NO: 838 - protein, 36 aa, synthetic construct; identical to SEQ ID NO: 814

SEQ ID NO: 839 - protein, 36 aa, synthetic construct; identical to SEQ ID NO: 814

SEQ ID NO: 840 - protein, 36 aa, synthetic construct; identical to SEQ ID NO: 814

SEQ ID NO: 841 - protein, 36 aa, synthetic construct; identical to SEQ ID NO: 814

SEQ ID NO: 842 - protein, 27 aa, synthetic construct
GGGGPOGPOGPOGPQGLLGAGGPOGPO

SEQ ID NO: 843 - protein, 27 aa, synthetic construct
GGGGPOGPOGPOGPQGLLGAGGPOGPO

SEQ ID NO: 844 - protein, 27 aa, synthetic construct
GGGGPOGPOGPOGPQGLLGAGGPOGPO

SEQ ID NO: 845 - protein, 27 aa, synthetic construct
GGGGPOGPOGPOGPQGLLGAGGPOGPO

SEQ ID NO: 846 - protein, 27 aa, synthetic construct
GGGGPOGPOGPOGPQGLLGAGGPOGPO

SEQ ID NO: 847 - protein, 27 aa, synthetic construct
GGGGPOGPOGPOGPQGLLGAGGPOGPO

SEQ ID NO: 848 - protein, 27 aa, synthetic construct
GGGGPOGPOGPOGPQGLLGAGGPOGPO

SEQ ID NO: 849 - protein, 27 aa, synthetic construct
GGGGPOGPOGPOGPQGLLGAGGPOGPO

SEQ ID NO: 850 - protein, 27 aa, synthetic construct
GGGGPOGPOGPOGPQGLLGAGGPOGPO

SEQ ID NO: 851 - protein, 27 aa, synthetic construct
GGGGPOGPOGPOGPQGLLGAGGPOGPO

SEQ ID NO: 852 - protein, 27 aa, synthetic construct
GGGGPOGPOGPOGPQGLLGAGGPOGPO

SEQ ID NO: 853 - protein, 27 aa, synthetic construct
GGGGPOGPOGPOGPQGLLGAGGPOGPO

SEQ ID NO: 854 - protein, 27 aa, synthetic construct
GGGGPOGPOGPOGPQGLLGAGGPOGPO

SEQ ID NO: 855 - protein, 27 aa, synthetic construct
GGGGPOGPOGPOGPQGLLGAGGPOGPO

SEQ ID NO: 856 - protein, 27 aa, synthetic construct
GGGGPOGPOGPOGPQGLLGAGGPOGPO

SEQ ID NO: 857 - protein, 27 aa, synthetic construct
GGGGPOGPOGPOGPQGLLGAGGPOGPO

SEQ ID NO: 858 - protein, 27 aa, synthetic construct
GGGGPOGPOGPOGPQGLLGAGGPOGPO

SEQ ID NO: 859 - protein, 27 aa, synthetic construct
GGGGPOGPOGPOGPQGLLGAGGPOGPO

SEQ ID NO: 860 - protein, 27 aa, synthetic construct
GGGGPOGPOGPOGPQGLLGAGGPOGPO

SEQ ID NO: 861 - protein, 27 aa, synthetic construct
GGGGPOGPOGPOGPQGLLGAGGPOGPO

SEQ ID NO: 862 - protein, 27 aa, synthetic construct
GGGGPOGPOGPOGPQGLLGAGGPOGPO

SEQ ID NO: 863 - protein, 27 aa, synthetic construct
GGGGPOGPOGPOGPQGLLGAGGPOGPO

SEQ ID NO: 864 - protein, 27 aa, synthetic construct
GGGGPOGPOGPOGPQGLLGAGGPOGPO

SEQ ID NO: 865 - protein, 27 aa, synthetic construct
GGGGPOGPOGPOGPQGLLGAGGPOGPO

SEQ ID NO: 866 - protein, 27 aa, synthetic construct
GGGGPOGPOGPOGPQGLLGAGGPOGPO

SEQ ID NO: 867 - protein, 27 aa, synthetic construct
GGGGPOGPOGPOGPQGLLGAGGPOGPO

SEQ ID NO: 868 - protein, 27 aa, synthetic construct
GGGGPOGPOGPOGPQGLLGAGGPOGPO

SEQ ID NO: 869 - protein, 27 aa, synthetic construct
GGGGPOGPOGPOGPQGLLGAGGPOGPO

SEQ ID NO: 870 - protein, 30 aa, synthetic construct
GGGGPOGPOGPOGPQGLLGAGGPOGPOGPO

SEQ ID NO: 871 - protein, 30 aa, synthetic construct; identical to SEQ ID NO: 870

SEQ ID NO: 872 - protein, 30 aa, synthetic construct; identical to SEQ ID NO: 870

SEQ ID NO: 873 - protein, 30 aa, synthetic construct; identical to SEQ ID NO: 870

SEQ ID NO: 874 - protein, 30 aa, synthetic construct; identical to SEQ ID NO: 870

SEQ ID NO: 875 - protein, 30 aa, synthetic construct; identical to SEQ ID NO: 870

SEQ ID NO: 876 - protein, 30 aa, synthetic construct; identical to SEQ ID NO: 870

SEQ ID NO: 877 - protein, 30 aa, synthetic construct; identical to SEQ ID NO: 870

SEQ ID NO: 878 - protein, 30 aa, synthetic construct; identical to SEQ ID NO: 870

SEQ ID NO: 879 - protein, 30 aa, synthetic construct; identical to SEQ ID NO: 870

SEQ ID NO: 880 - protein, 30 aa, synthetic construct; identical to SEQ ID NO: 870

SEQ ID NO: 881 - protein, 30 aa, synthetic construct; identical to SEQ ID NO: 870

SEQ ID NO: 882 - protein, 30 aa, synthetic construct; identical to SEQ ID NO: 870

SEQ ID NO: 883 - protein, 30 aa, synthetic construct; identical to SEQ ID NO: 870

SEQ ID NO: 884 - protein, 30 aa, synthetic construct; identical to SEQ ID NO: 870

SEQ ID NO: 885 - protein, 30 aa, synthetic construct; identical to SEQ ID NO: 870

SEQ ID NO: 886 - protein, 30 aa, synthetic construct; identical to SEQ ID NO: 870

SEQ ID NO: 887 - protein, 30 aa, synthetic construct; identical to SEQ ID NO: 870

SEQ ID NO: 888 - protein, 30 aa, synthetic construct; identical to SEQ ID NO: 870

SEQ ID NO: 889 - protein, 30 aa, synthetic construct; identical to SEQ ID NO: 870

SEQ ID NO: 890 - protein, 30 aa, synthetic construct; identical to SEQ ID NO: 870

SEQ ID NO: 891 - protein, 30 aa, synthetic construct; identical to SEQ ID NO: 870

SEQ ID NO: 892 - protein, 30 aa, synthetic construct; identical to SEQ ID NO: 870

SEQ ID NO: 893 - protein, 30 aa, synthetic construct; identical to SEQ ID NO: 870

SEQ ID NO: 894 - protein, 30 aa, synthetic construct; identical to SEQ ID NO: 870

SEQ ID NO: 895 - protein, 30 aa, synthetic construct; identical to SEQ ID NO: 870

SEQ ID NO: 896 - protein, 30 aa, synthetic construct; identical to SEQ ID NO: 870

SEQ ID NO: 897 - protein, 30 aa, synthetic construct; identical to SEQ ID NO: 870

SEQ ID NO: 898 - protein, 30 aa, synthetic construct
GGGGFOGFOGFOGPQGLLGAGGPOGPOGPO

SEQ ID NO: 899 - protein, 30 aa, synthetic construct; identical to SEQ ID NO: 898

SEQ ID NO: 900 - protein, 30 aa, synthetic construct; identical to SEQ ID NO: 898

SEQ ID NO: 901 - protein, 30 aa, synthetic construct; identical to SEQ ID NO: 898

SEQ ID NO: 902 - protein, 30 aa, synthetic construct; identical to SEQ ID NO: 898

SEQ ID NO: 903 - protein, 30 aa, synthetic construct; identical to SEQ ID NO: 898

SEQ ID NO: 904 - protein, 30 aa, synthetic construct; identical to SEQ ID NO: 898

SEQ ID NO: 905 - protein, 30 aa, synthetic construct; identical to SEQ ID NO: 898

SEQ ID NO: 906 - protein, 30 aa, synthetic construct; identical to SEQ ID NO: 898

SEQ ID NO: 907 - protein, 30 aa, synthetic construct; identical to SEQ ID NO: 898

SEQ ID NO: 908 - protein, 30 aa, synthetic construct; identical to SEQ ID NO: 898

SEQ ID NO: 909 - protein, 30 aa, synthetic construct; identical to SEQ ID NO: 898

SEQ ID NO: 910 - protein, 30 aa, synthetic construct; identical to SEQ ID NO: 898

SEQ ID NO: 911 - protein, 30 aa, synthetic construct; identical to SEQ ID NO: 898

SEQ ID NO: 912 - protein, 30 aa, synthetic construct; identical to SEQ ID NO: 898

SEQ ID NO: 913 - protein, 30 aa, synthetic construct; identical to SEQ ID NO: 898

SEQ ID NO: 914 - protein, 30 aa, synthetic construct; identical to SEQ ID NO: 898

SEQ ID NO: 915 - protein, 30 aa, synthetic construct; identical to SEQ ID NO: 898

SEQ ID NO: 916 - protein, 30 aa, synthetic construct; identical to SEQ ID NO: 898

SEQ ID NO: 917 - protein, 30 aa, synthetic construct; identical to SEQ ID NO: 898

SEQ ID NO: 918 - protein, 30 aa, synthetic construct; identical to SEQ ID NO: 898

SEQ ID NO: 919 - protein, 30 aa, synthetic construct; identical to SEQ ID NO: 898

SEQ ID NO: 920 - protein, 30 aa, synthetic construct; identical to SEQ ID NO: 898

SEQ ID NO: 921 - protein, 30 aa, synthetic construct; identical to SEQ ID NO: 898

SEQ ID NO: 922 - protein, 30 aa, synthetic construct; identical to SEQ ID NO: 898

SEQ ID NO: 923 - protein, 30 aa, synthetic construct; identical to SEQ ID NO: 898

SEQ ID NO: 924 - protein, 30 aa, synthetic construct; identical to SEQ ID NO: 898

SEQ ID NO: 925 - protein, 30 aa, synthetic construct; identical to SEQ ID NO: 898

SEQ ID NO: 926 - protein, 27 aa, synthetic construct
GGGGFOGFOGFOGPQGLLGAGGPOGPO

SEQ ID NO: 927 - protein, 27 aa, synthetic construct
GGGGFOGFOGFOGPQGLLGAGGPOGPO

SEQ ID NO: 928 - protein, 27 aa, synthetic construct
GGGGFOGFOGFOGPQGLLGAGGPOGPO

SEQ ID NO: 929 - protein, 27 aa, synthetic construct
GGGGFOGFOGFOGPQGLLGAGGPOGPO

SEQ ID NO: 930 - protein, 27 aa, synthetic construct
GGGGFOGFOGFOGPQGLLGAGGPOGPO

SEQ ID NO: 931 - protein, 27 aa, synthetic construct
GGGGFOGFOGFOGPQGLLGAGGPOGPO

SEQ ID NO: 932 - protein, 27 aa, synthetic construct
GGGGFOGFOGFOGPQGLLGAGGPOGPO

SEQ ID NO: 933 - protein, 27 aa, synthetic construct
GGGGFOGFOGFOGPQGLLGAGGPOGPO

SEQ ID NO: 934 - protein, 27 aa, synthetic construct
GGGGFOGFOGFOGPQGLLGAGGPOGPO

SEQ ID NO: 935 - protein, 27 aa, synthetic construct
GGGGFOGFOGFOGPQGLLGAGGPOGPO

SEQ ID NO: 936 - protein, 27 aa, synthetic construct
GGGGFOGFOGFOGPQGLLGAGGPOGPO

SEQ ID NO: 937 - protein, 27 aa, synthetic construct
GGGGFOGFOGFOGPQGLLGAGGPOGPO

SEQ ID NO: 938 - protein, 27 aa, synthetic construct
GGGGFOGFOGFOGPQGLLGAGGPOGPO

SEQ ID NO: 939 - protein, 27 aa, synthetic construct
GGGGFOGFOGFOGPQGLLGAGGPOGPO

SEQ ID NO: 940 - protein, 27 aa, synthetic construct
GGGGFOGFOGFOGPQGLLGAGGPOGPO

SEQ ID NO: 941 - protein, 27 aa, synthetic construct
GGGGFOGFOGFOGPQGLLGAGGPOGPO

SEQ ID NO: 942 - protein, 27 aa, synthetic construct
GGGGFOGFOGFOGPQGLLGAGGPOGPO

SEQ ID NO: 943 - protein, 27 aa, synthetic construct
GGGGFOGFOGFOGPQGLLGAGGPOGPO

SEQ ID NO: 944 - protein, 27 aa, synthetic construct
GGGGFOGFOGFOGPQGLLGAGGPOGPO

SEQ ID NO: 945 - protein, 27 aa, synthetic construct
GGGGFOGFOGFOGPQGLLGAGGPOGPO

SEQ ID NO: 946 - protein, 27 aa, synthetic construct
GGGGFOGFOGFOGPQGLLGAGGPOGPO

SEQ ID NO: 947 - protein, 27 aa, synthetic construct
GGGGFOGFOGFOGPQGLLGAGGPOGPO

SEQ ID NO: 948 - protein, 27 aa, synthetic construct
GGGGFOGFOGFOGPQGLLGAGGPOGPO

SEQ ID NO: 949 - protein, 27 aa, synthetic construct
GGGGFOGFOGFOGPQGLLGAGGPOGPO

SEQ ID NO: 950 - protein, 27 aa, synthetic construct
GGGGFOGFOGFOGPQGLLGAGGPOGPO

SEQ ID NO: 951 - protein, 27 aa, synthetic construct
GGGGFOGFOGFOGPQGLLGAGGPOGPO

SEQ ID NO: 952 - protein, 27 aa, synthetic construct
GGGGFOGFOGFOGPQGLLGAGGPOGPO

SEQ ID NO: 953 - protein, 27 aa, synthetic construct
GGGGFOGFOGFOGPQGLLGAGGPOGPO

SEQ ID NO: 954 - protein, 27 aa, synthetic construct
GGGGFOGFOGFOGPQGLLGAGGFOGFO

SEQ ID NO: 955 - protein, 27 aa, synthetic construct
GGGGFOGFOGFOGPQGLLGAGGFOGFO

SEQ ID NO: 956 - protein, 27 aa, synthetic construct
GGGGFOGFOGFOGPQGLLGAGGFOGFO

SEQ ID NO: 957 - protein, 27 aa, synthetic construct
GGGGFOGFOGFOGPQGLLGAGGFOGFO

SEQ ID NO: 958 - protein, 27 aa, synthetic construct
GGGGFOGFOGFOGPQGLLGAGGFOGFO

SEQ ID NO: 959 - protein, 27 aa, synthetic construct
GGGGFOGFOGFOGPQGLLGAGGFOGFO

SEQ ID NO: 960 - protein, 27 aa, synthetic construct
GGGGFOGFOGFOGPQGLLGAGGFOGFO

SEQ ID NO: 961 - protein, 27 aa, synthetic construct
GGGGFOGFOGFOGPQGLLGAGGFOGFO

SEQ ID NO: 962 - protein, 27 aa, synthetic construct
GGGGFOGFOGFOGPQGLLGAGGFOGFO

SEQ ID NO: 963 - protein, 27 aa, synthetic construct
GGGGFOGFOGFOGPQGLLGAGGFOGFO

SEQ ID NO: 964 - protein, 27 aa, synthetic construct
GGGGFOGFOGFOGPQGLLGAGGFOGFO

SEQ ID NO: 965 - protein, 27 aa, synthetic construct
GGGGFOGFOGFOGPQGLLGAGGFOGFO

SEQ ID NO: 966 - protein, 27 aa, synthetic construct
GGGGFOGFOGFOGPQGLLGAGGFOGFO

SEQ ID NO: 967 - protein, 27 aa, synthetic construct
GGGGFOGFOGFOGPQGLLGAGGFOGFO

SEQ ID NO: 968 - protein, 27 aa, synthetic construct
GGGGFOGFOGFOGPQGLLGAGGFOGFO

SEQ ID NO: 969 - protein, 27 aa, synthetic construct
GGGGFOGFOGFOGPQGLLGAGGFOGFO

SEQ ID NO: 970 - protein, 27 aa, synthetic construct
GGGGFOGFOGFOGPQGLLGAGGFOGFO

SEQ ID NO: 971 - protein, 27 aa, synthetic construct
GGGGFOGFOGFOGPQGLLGAGGFOGFO

SEQ ID NO: 972 - protein, 27 aa, synthetic construct
GGGGFOGFOGFOGPQGLLGAGGFOGFO

SEQ ID NO: 973 - protein, 27 aa, synthetic construct
GGGGFOGFOGFOGPQGLLGAGGFOGFO

SEQ ID NO: 974 - protein, 27 aa, synthetic construct
GGGGFOGFOGFOGPQGLLGAGGFOGFO

SEQ ID NO: 975 - protein, 27 aa, synthetic construct
GGGGFOGFOGFOGPQGLLGAGGFOGFO

SEQ ID NO: 976 - protein, 27 aa, synthetic construct
GGGGFOGFOGFOGPQGLLGAGGFOGFO

SEQ ID NO: 977 - protein, 27 aa, synthetic construct
GGGGFOGFOGFOGPQGLLGAGGFOGFO

SEQ ID NO: 978 - protein, 27 aa, synthetic construct
GGGGFOGFOGFOGPQGLLGAGGFOGFO

SEQ ID NO: 979 - protein, 27 aa, synthetic construct
GGGGFOGFOGFOGPQGLLGAGGFOGFO

SEQ ID NO: 980 - protein, 27 aa, synthetic construct
GGGGFOGFOGFOGPQGLLGAGGFOGFO

SEQ ID NO: 981 - protein, 27 aa, synthetic construct
GGGGFOGFOGFOGPQGLLGAGGFOGFO

SEQ ID NO: 982 - protein, 30 aa, synthetic construct
GGGGFOGFOGFOGPQGLLGAGGFOGFOGFO

SEQ ID NO: 983 - protein, 30 aa, synthetic construct; identical to SEQ ID NO: 982

SEQ ID NO: 984 - protein, 30 aa, synthetic construct; identical to SEQ ID NO: 982

SEQ ID NO: 985 - protein, 30 aa, synthetic construct; identical to SEQ ID NO: 982

SEQ ID NO: 986 - protein, 30 aa, synthetic construct; identical to SEQ ID NO: 982

SEQ ID NO: 987 - protein, 30 aa, synthetic construct; identical to SEQ ID NO: 982

SEQ ID NO: 988 - protein, 30 aa, synthetic construct; identical to SEQ ID NO: 982

SEQ ID NO: 989 - protein, 30 aa, synthetic construct; identical to SEQ ID NO: 982

SEQ ID NO: 990 - protein, 30 aa, synthetic construct; identical to SEQ ID NO: 982

SEQ ID NO: 991 - protein, 30 aa, synthetic construct; identical to SEQ ID NO: 982

SEQ ID NO: 992 - protein, 30 aa, synthetic construct; identical to SEQ ID NO: 982

SEQ ID NO: 993 - protein, 30 aa, synthetic construct; identical to SEQ ID NO: 982

SEQ ID NO: 994 - protein, 30 aa, synthetic construct; identical to SEQ ID NO: 982

SEQ ID NO: 995 - protein, 30 aa, synthetic construct; identical to SEQ ID NO: 982

SEQ ID NO: 996 - protein, 30 aa, synthetic construct; identical to SEQ ID NO: 982

SEQ ID NO: 997 - protein, 30 aa, synthetic construct; identical to SEQ ID NO: 982

SEQ ID NO: 998 - protein, 30 aa, synthetic construct; identical to SEQ ID NO: 982

SEQ ID NO: 999 - protein, 30 aa, synthetic construct; identical to SEQ ID NO: 982

SEQ ID NO: 1000 - protein, 30 aa, synthetic construct; identical to SEQ ID NO: 982

SEQ ID NO: 1001 - protein, 30 aa, synthetic construct; identical to SEQ ID NO: 982

SEQ ID NO: 1002 - protein, 30 aa, synthetic construct; identical to SEQ ID NO: 982

SEQ ID NO: 1003 - protein, 30 aa, synthetic construct; identical to SEQ ID NO: 982

SEQ ID NO: 1004 - protein, 30 aa, synthetic construct; identical to SEQ ID NO: 982

SEQ ID NO: 1005 - protein, 30 aa, synthetic construct; identical to SEQ ID NO: 982

SEQ ID NO: 1006 - protein, 30 aa, synthetic construct; identical to SEQ ID NO: 982

SEQ ID NO: 1007 - protein, 30 aa, synthetic construct; identical to SEQ ID NO: 982

SEQ ID NO: 1008 - protein, 30 aa, synthetic construct; identical to SEQ ID NO: 982

SEQ ID NO: 1009 - protein, 30 aa, synthetic construct; identical to SEQ ID NO: 982